(19) United States
(12) Patent Application Publication
(10) Pub. No.: US 2024/0076744 A1
(43) Pub. Date: Mar. 7, 2024

(54) METHODS AND SYSTEMS FOR mRNA BOUNDARY ANALYSIS IN NEXT GENERATION SEQUENCING

(71) Applicant: Tempus Labs, Inc., Chicago, IL (US)

(72) Inventors: Richard Blidner, Glenview, IL (US); Eric Leon Harness, Chicago, IL (US)

(21) Appl. No.: 18/261,985

(22) PCT Filed: Jan. 21, 2022
(86) PCT No.: PCT/US2022/013421

§ 371 Date: Jul. 18, 2023

Related U.S. Application Data
(60) Provisional application No. 63/139,994, filed on Jan. 21, 2021.
(60) Provisional application No. 63/167,490, filed on Mar. 29, 2021.
(60) Provisional application No. 63/167,494, filed on Mar. 29, 2021.

(51) Int. Cl. C12Q 1/6886; G06N 20/10; G16B 20/20; G16B 25/10; G16B 40/20; G16H 20/00; G16H 50/20

(52) U.S. Cl. CPC C12Q 1/6886; G06N 20/10; G16B 20/20; G16B 25/10; G16B 40/20; G16H 20/00; G16H 50/20; C12Q 2600/106; C12Q 2600/112; C12Q 2600/156

(57) ABSTRACT

Methods, systems, and software are provided for detecting gene fusions in a subject with a cancer condition through mRNA boundary analysis of next generation sequencing of a transcriptome or relevant part thereof. Methods, systems, and software are provided for detecting splice variants in a subject with a cancer condition through mRNA boundary analysis of next generation sequencing of a transcriptome or relevant part thereof. Methods, systems, and software are provided for evaluating the complexity of an RNA-seq sequencing reaction through mRNA boundary analysis. Generally, the methods described herein include obtaining sequences of mRNA molecules for a plurality of genes in a sample of a subject. For each gene, an RNA boundary distribution including relative abundance value for each respective RNA boundary sub-sequence of the gene is determined from the plurality of sequences. These abundance values are evaluated using one or more models to provide the analyses described herein.

### CROSS REFERENCE TO RELATED APPLICATIONS

This application claims priority to U.S. Provisional Patent Application No. 63/139,994, filed Jan. 21, 2021, U.S. Provisional Patent Application No. 63/167,490, filed Mar. 29, 2021, and U.S. Provisional Patent Application No. 63/167,494, filed Mar. 29, 2021, the contents of which are hereby incorporated by reference, in their entireties, for all purposes.

### FIELD OF THE INVENTION

The present disclosure relates generally to the quantification of various types of boundary sequences in mRNA sequencing data to characterize the data set, identify mRNA species of interest, and/or evaluate an mRNA expression pattern of a tissue.

### BACKGROUND

Precision oncology is the practice of tailoring cancer therapy to the unique genomic, epigenetic, and/or transcriptomic profile of an individual's cancer. Personalized cancer treatment builds upon conventional therapeutic regimens used to treat cancer based only on the gross classification of the cancer, e.g., treating all breast cancer patients with a first therapy and all lung cancer patients with a second therapy. This field was borne out of many observations that different patients diagnosed with the same type of cancer, e.g., breast cancer, responded very differently to common treatment regimens. Over time, researchers have identified genomic, epigenetic, and transcriptomic markers that improve predictions as to how an individual cancer will respond to a particular treatment modality.

There is growing evidence that cancer patients who receive therapy guided by their genetics have better outcomes. For example, studies have shown that targeted therapies result in significantly improved progression-free cancer survival. See, e.g., Radovich M. et al., Oncotarget, 7(35):56491-500 (2016). Similarly, reports from the IMPACT trial—a large (n=1307) retrospective analysis of consecutive, prospectively molecularly profiled patients with advanced cancer who participated in a large, personalized medicine trial—indicate that patients receiving targeted therapies matched to their tumor biology had a response rate of 16.2%, as opposed to a response rate of 5.2% for patients receiving non-matched therapy. Tsimberidou A M et al., ASCO 2018, Abstract LBA2553 (2018).

In fact, therapy targeted to specific genomic alterations is already the standard of care in several tumor types, e.g., as suggested in the National Comprehensive Cancer Network (NCCN) guidelines for melanoma, colorectal cancer, and non-small cell lung cancer. In practice, implementation of these targeted therapies requires determining the status of the diagnostic marker in each eligible cancer patient. While this can be accomplished for the few, well known mutations associated with treatment recommendations in the NCCN guidelines using individual assays or small next generation sequencing (NGS) panels, the growing number of actionable genomic alterations and increasing complexity of diagnostic classifiers necessitates a more comprehensive evaluation of each patient's cancer genome, epigenome, and/or transcriptome.

For instance, some evidence suggests that use of combination therapies where each component is matched to an actionable genomic alteration holds the greatest potential for treating individual cancers. To this point, a retroactive study of cancer patients treated with one or more therapeutic regimens revealed that patients who received therapies matched to a higher percentage of their genomic alterations experienced a greater frequency of stable disease (e.g., a longer time to recurrence), longer time to treatment failure, and greater overall survival. Wheeler J J et al., Cancer Res., 76:3690-701 (2016). Thus, comprehensive evaluation of each cancer patient's genome, epigenome, and/or transcriptome should maximize the benefits provided by precision oncology, by facilitating more fine-tuned combination therapies, use of novel off-label drug indications, and/or tissue agnostic immunotherapy. See, for example, Schwaederle M. et al., J Clin Oncol., 33(32):3817-25 (2015); Schwaederle M. et al., JAMA Oncol., 2(11):1452-59 (2016); and Wheler J J et al., Cancer Res., 76(13):3690-701 (2016). Further, the use of comprehensive next generation sequencing analysis of cancer genomes facilitates better access and a larger patient pool for clinical trial enrollment. Coyne G O et al., Curr. Probl. Cancer, 41(3):182-93 (2017); and Markman M., Oncology, 31(3):158, 168.

The use of large NGS genomic analysis is growing in order to address the need for more comprehensive characterization of an individual's cancer genome. See, for example, Fernandes G S et al., Clinics, 72(10):588-94. Recent studies indicate that of the patients for which large NGS genomic analysis is performed, 30-40% then receive clinical care based on the assay results, which is limited by at least the identification of actionable genomic alterations, the availability of medication for treatment of identified actionable genomic alterations, and the clinical condition of the subject. See, Ross J S et al., JAMA Oncol., 1(1):40-49 (2015); Ross J S et al., Arch. Pathol. Lab Med., 139:642-49 (2015); Hirshfield K M et al., Oncologist, 21(11):1315-25 (2016); and Groisberg R. et al., Oncotarget, 8:39254-67 (2017).

However, RNA expression profiling is hampered by inconsistent results stemming from a variety of sources, including variable sample quality, variable library preparation quality, and variable sequencing quality. For instance, the method of tissue collection, preservation (e.g., formalin fixation), and/or storage of tissue biopsies, as well as the methodology used to extract RNA therefrom, can result in sample degradation and variable quality of the sequencing library. This, in turn, leads to inaccuracies in downstream assays and analysis, including next-generation sequencing (NGS) for the identification of biomarkers. Ilie and Hofman, Transl Lung Cancer Res., 5(4):420-23 (2016).

Moreover, insufficient methods and metrics are available for assessing the sensitivity and level of detection (LOD) of next generation RNA sequencing reactions due, at least in part, to the inherent variability in expression between targets within the same class and variation associated with input amount of total or enriched RNA. For example, it is difficult to determine whether the absence or low relative abundance of a particular mRNA target in an RNA sequencing reaction is due to a biological difference in the expression pattern of a subject's exome or to poor quality and/or a low diversity of nucleic acid fragments in the sample being sequenced.

The information disclosed in this Background section is only for enhancement of understanding of the general background of the invention and should not be taken as an acknowledgement or any form of suggestion that this information forms the prior art already known to a person skilled in the art.

### SUMMARY

Given the above background, there is a need in the art for improved metrics, methods, and systems for evaluating the sensitivity and level of detection of mRNA sequencing assays, e.g., transcriptional profiling assays. Advantageously, the present disclosure solves this and other needs in the art by providing improved methodologies for determining the sensitivity and/or LOD in an RNA sequencing assay through evaluation of various boundary sequences e.g., exon/exon boundaries, gene fusion boundaries, and/or indel boundaries, in RNA sequencing results. Accordingly, the present disclosure provides a dynamic model of how to assess detection sensitivity of a given target, as well as methods for developing such models.

Advantageously, the methods and systems described herein can be used to assess the sensitivity of an RNA transcriptome analysis, e.g., performed using next generation sequencing, for various classes of targets including relative RNA expression LOD, RNA fusion LOD and sensitivity, and RNA splice variant LOD and sensitivity. The models described herein can also be used to assess the range of sample quality, e.g., quality variation among FFPE sample and sample cohorts, and range of sample input required to meet a desired sensitivity and LOD.

Accordingly, various aspects of the present disclosure facilitate better models of the performance and limitations of mRNA sequencing assays, which can be used to better inform patients and clinicians. Various aspects of the disclosure also facilitate the ability to recover useful information from lower-quality samples. Likewise, various aspects of the disclosure allow improved confidence in sequencing results showing high expressing targets from a low quality or low abundant sample.

In some embodiments, RNA target data is evaluated as a set of boundary transitions. For example, mRNA expression can be represented by exon-exon boundaries, fusion detection can be represented as fusion breakpoints, and splice variant detection can be represented as unique splice boundaries.

As will be appreciated, these targets exist in a vast excess of other RNA fragments. The ability to detect a particular species is a function of the relative abundance of the target species and the total amount of starting material. Within a sample, the relative amount of target stays the same with decreasing amounts of total material. However, decreasing the relative diversity of fragments and abundance of unique targets in a sequencing reaction—e.g., by using a lower amount of input material in the sequencing reaction or due to increased damage to RNA in the sample, thereby reducing the amount of functional material suitable for library preparation and amplification—will increase the likelihood of not observing the target species in the sequencing results.

This is particularly true for target species present at lower relative abundances in a sample. That is, for target species at lower relative abundance, sampling errors are more magnified as sample complexity decreases than for target species at higher relative abundance in the sample. For instance, as illustrated in FIG. 8, assume that a target species present in a sample at a relative amount of 1 transcript per ten million transcripts (0.1 transcripts per million (TPM)) has a drop-out rate of 1% when sequenced in a reaction having a first sample complexity. Reducing the sample complexity of the sequencing reaction by a particular amount will increase the drop-out rate for the target species to 10%. In contrast, for a target species present in a sample at a relative concentration of 10 transcripts per ten million transcripts (1 TPM), reducing the complexity of a sequencing reaction that yields a 1% drop out rate for the target species by the same particular amount will only increase the drop-out rate of the target species to 2% (rather than 10%).

This is also shown in FIG. 9, which illustrates theoretical detection of a species present at different relative abundance values (FIG. 9A) or in samples of different quality (FIG. 9B), with increasing amounts of input material for the sequencing reaction. As illustrated in FIG. 10, the number of exon boundaries for mRNA species in a sequencing reaction can be used to evaluate the total diversity of fragments in the sequencing reaction. For instance, when more boundary sequences of an RNA species present at a particular relative abundance are observed in a first sequencing reaction than in a second sequencing reaction, it can be concluded that the input for the first sequencing reaction is of higher quality or higher fragment diversity.

Accordingly, in some embodiments, a model of nucleic acid sample diversity and/or quality can be built based on, e.g., reported expression of mRNA species in a universal human reference (UHR) sample, on quantitative PCR (qPCR) data, and/or digital droplet PCR (ddPCR) data.

In some embodiments, a range of abundance (e.g., TPM) bins are established, as illustrated in FIG. 11. RNA expression of a sample is measured, e.g., using replicate sequencing reactions. In some embodiments, all genes within a known abundance range, e.g., from published literature on UHR analysis, are analyzed to build the model. In some embodiments, randomly suggested bins of 20-50 genes are established and the analysis is repeated, e.g., using in silico sampling). In some embodiments, a set of target genes are selected in each bin (e.g., 20 genes in each bin), and the abundance of these genes is determined by PCR.

Generally, each transcript counts as a measurement point for that range, such that transcripts are measured categorically, e.g., 20 samples. The analysis can be run on clinical samples, and the effects of FFPE mediated degradation on transcript detection can be characterized. In this fashion, LOD and LOQ can be validated.

In some embodiments, an initial range over which LOD and LOQ are linearly related to the diversity of the sample and/or quality of the sample can be determined. In some embodiments, a secondary range of linearity can be determined based on differential expression of a gene subset.

In some embodiments, a set of boundary transitions for two genes is used to determine whether a gene fusion between the two genes is present in a tissue of the subject. For example, in some embodiments, a plurality of sequences of mRNA molecules for a plurality of genes in a sample of the patient are obtained. For each gene, an RNA boundary distribution including relative abundance value for each respective RNA boundary sub-sequence of the gene is determined from the plurality of sequences. For a pair of genes, the RNA boundary distributions for the two genes in the pair of genes are evaluated with a model that has been trained to detect gene fusions based on RNA boundary distributions. An indication of whether a gene fusion between the pair of genes is present in the first tissue is determined based on the output of the model.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B, 1C, and 1D collectively illustrate a block diagram of an example computing device for mRNA boundary analysis in next generation sequencing, in accordance with some embodiments of the present disclosure.

FIG. 2A illustrates an example workflow for generating a clinical report based on information generated from analysis of one or more patient specimens, in accordance with some embodiments of the present disclosure.

FIG. 2B illustrates an example of a distributed diagnostic environment for collecting and evaluating patient data for the purpose of precision medicine, in accordance with some embodiments of the present disclosure.

FIG. 3 provides an example flow chart of processes and features for sample collection and analysis for use in precision medicine, in accordance with some embodiments of the present disclosure.

FIGS. 4A, 4B, and 4C collectively illustrate an example bioinformatics pipeline for mRNA boundary analysis in next generation sequencing, in accordance with some embodiments of the present disclosure. FIG. 4A provides an overview flow chart of processes and features in a bioinformatics pipeline, in accordance with some embodiments of the present disclosure.

FIG. 4B illustrates an example flow chart of processes and features for determining a genetic status of a subject through mRNA boundary analysis, in which dashed boxes represent optional portions of the method, in accordance with some embodiments of the present disclosure. FIG. 4C illustrates an example flow chart of processes and features for evaluating the nucleic acid complexity of a nucleic acid sequencing reaction through mRNA boundary analysis, in which dashed boxes represent optional portions of the method, in accordance with some embodiments of the present disclosure.

FIGS. 5A, 5B, 5C, 5D, 5E, 5F, and 5G collectively provide a flow chart of processes and features for determining a genetic status of a subject through mRNA boundary analysis, in which dashed boxes represent optional portions of the method, in accordance with some embodiments of the present disclosure.

FIGS. 6A and 6B collectively illustrate an example of mRNA boundary analysis for a plurality of differentially spliced mRNA isoforms of a gene (FIG. 6A) and the resulting boundary element counts (FIG. 6B), in accordance with some embodiments of the present disclosure.

FIGS. 7A, 7B, 7C, and 7D collectively provide a flow chart of processes and features for evaluating the nucleic acid complexity of a nucleic acid sequencing reaction through mRNA boundary analysis, in which dashed boxes represent optional portions of the method, in accordance with some embodiments of the present disclosure.

FIG. 8 illustrates that sampling error for a target increases with decreasing relative abundance of that target.

FIGS. 9A and 9B collectively illustrate that increasing amounts of sequencing input material is needed to detect targets at the same detection sensitivity as the relative abundance of that target decreases (FIG. 9A) and/or the quality of the sample decreases (FIG. 9B).

FIG. 10 illustrates the ability to correlate the number of exon boundaries detected with a quality of the sequencing sample, in accordance with some embodiments of the present disclosure.

FIG. 11 illustrates binning of targets present at different relative abundances in order to establish a curve correlating expression data with transcript abundance, in accordance with some embodiments of the present disclosure.

Like reference numerals refer to corresponding parts throughout the several views of the drawings.

### DETAILED DESCRIPTION

### Introduction

The present disclosure provides methods and systems for assessing the sensitivity of an RNA transcriptome analysis, e.g., performed using next generation sequencing, for various classes of targets including relative RNA expression LOD, RNA fusion LOD and sensitivity, and RNA splice variant LOD and sensitivity. The models described herein can also be used to assess the range of sample quality, e.g., quality variation among FFPE sample and sample cohorts, and range of sample input required to meet a desired sensitivity and LOD. Advantageously, in some embodiments, the methods and systems described herein improve determination of a genetic status of a subject and/or improve evaluation of the nucleic acid complexity of a nucleic acid sequencing reaction, through various forms of mRNA boundary analysis. In some embodiments, the mRNA boundary analysis considers one or more of exon-exon boundary elements, exon-intron boundary elements, intron-intron boundary elements, intron-flanking nucleic acid boundary elements, and intron-noncoding sequence boundary elements.

### Definitions

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. Furthermore, the terms “subject,” “user,” and “patient” are used interchangeably herein.

As used herein, the term “subject” refers to any living or non-living organism including, but not limited to, a human (e.g., a male human, female human, fetus, pregnant female, child, or the like), a non-human mammal, or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a woman, or a child).

As used herein, the terms “control,” “control sample,” “reference,” “reference sample,” “normal,” and “normal sample” describe a sample from a non-diseased tissue. In some embodiments, such a sample is from a subject that does not have a particular condition (e.g., cancer). In other embodiments, such a sample is an internal control from a subject, e.g., who may or may not have the particular disease (e.g., cancer), but is from a healthy tissue of the subject. For example, where a liquid or solid tumor sample is obtained from a subject with cancer, an internal control sample may be obtained from a healthy tissue of the subject, e.g., a white blood cell sample from a subject without a blood cancer or a solid germline tissue sample from the subject. Accordingly, a reference sample can be obtained from the subject or from a database, e.g., from a second subject who does not have the particular disease (e.g., cancer).

As used herein the term “cancer,” “cancerous tissue,” or “tumor” refers to an abnormal mass of tissue in which the growth of the mass surpasses, and is not coordinated with, the growth of normal tissue, including both solid masses (e.g., as in a solid tumor) or fluid masses (e.g., as in a hematological cancer). A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue. Accordingly, a “tumor sample” refers to a biological sample obtained or derived from a tumor of a subject, as described herein.

Non-limiting examples of cancer types include ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, breast cancer, non-clear cell renal cell carcinoma, glioblastoma, glioma, kidney cancer, gastrointestinal stromal tumor, medulloblastoma, bladder cancer, gastric cancer, bone cancer, non-small cell lung cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, Her2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and papillary renal cell carcinoma.

As used herein, the terms “cancer state” or “cancer condition” refer to a characteristic of a cancer patient's condition, e.g., a diagnostic status, a type of cancer, a location of cancer, a primary origin of a cancer, a cancer stage, a cancer prognosis, and/or one or more additional characteristics of a cancer (e.g., tumor characteristics such as morphology, heterogeneity, size, etc.). In some embodiments, one or more additional personal characteristics of the subject are used further describe the cancer state or cancer condition of the subject, e.g., age, gender, weight, race, personal habits (e.g., smoking, drinking, diet), other pertinent medical conditions (e.g., high blood pressure, dry skin, other diseases), current medications, allergies, pertinent medical history, current side effects of cancer treatments and other medications, etc.

As used herein, the term “liquid biopsy” sample refers to any sample taken from a subject, which can reflect a biological state associated with the subject, and that includes cell-free DNA. Examples of liquid biopsy samples include, but are not limited to, blood, whole blood, plasma, serum, urine, cerebrospinal fluid, fecal, saliva, sweat, tears, pleural fluid, pericardial fluid, or peritoneal fluid of the subject. A liquid biopsy sample can include any tissue or material derived from a living or dead subject. A liquid biopsy sample can be a cell-free sample. A liquid biopsy sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof.

As used herein, the term “cell-free DNA” and “cfDNA” interchangeably refer to DNA fragments that circulate in a subject's body (e.g., bloodstream) and originate from one or more healthy cells and/or from one or more cancer cells. These DNA molecules are found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject, and are believed to be fragments of genomic DNA expelled from healthy and/or cancerous cells, e.g., upon apoptosis and lysis of the cellular envelope.

As used herein, the term “locus” refers to a position (e.g., a site) within a genome, e.g., on a particular chromosome. In some embodiments, a locus refers to a single nucleotide position, on a particular chromosome, within a genome. In some embodiments, a locus refers to a group of nucleotide positions within a genome. In some instances, a locus is defined by a mutation (e.g., substitution, insertion, deletion, inversion, or translocation) of consecutive nucleotides within a cancer genome. In some instances, a locus is defined by a gene, a sub-genic structure (e.g., a regulatory element, exon, intron, or combination thereof), or a predefined span of a chromosome. Because normal mammalian cells have diploid genomes, a normal mammalian genome (e.g., a human genome) will generally have two copies of every locus in the genome, or at least two copies of every locus located on the autosomal chromosomes, e.g., one copy on the maternal autosomal chromosome and one copy on the paternal autosomal chromosome.

As used herein, the term “allele” refers to a particular sequence of one or more nucleotides at a chromosomal locus. In a haploid organism, the subject has one allele at every chromosomal locus. In a diploid organism, the subject has two alleles at every chromosomal locus.

As used herein, the term “base pair” or “bp” refers to a unit consisting of two nucleobases bound to each other by hydrogen bonds. Generally, the size of an organism's genome is measured in base pairs because DNA is typically double stranded. However, some viruses have single-stranded DNA or RNA genomes.

As used herein, the terms “genomic alteration,” “mutation,” and “variant” refer to a detectable change in the genetic material of one or more cells. A genomic alteration, mutation, or variant can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene, or a large span of a chromosome) (CNV), a partial or complete change in the ploidy of the cell, as well as in changes in the epigenetic information of a genome, such as altered DNA methylation patterns. In some embodiments, a mutation is a change in the genetic information of the cell relative to a particular reference genome, or one or more ‘normal’ alleles found in the population of the species of the subject. For instance, mutations can be found in both germline cells (e.g., non-cancerous, ‘normal’ cells) of a subject and in abnormal cells (e.g., pre-cancerous or cancerous cells) of the subject. As such, a mutation in a germline of the subject (e.g., which is found in substantially all ‘normal cells’ in the subject) is identified relative to a reference genome for the species of the subject. However, many loci of a reference genome of a species are associated with several variant alleles that are significantly represented in the population of the subject and are not associated with a diseased state, e.g., such that they would not be considered ‘mutations.’ By contrast, in some embodiments, a mutation in a cancerous cell of a subject can be identified relative to either a reference genome of the subject or to the subject's own germline genome. In certain instances, identification of both types of variants can be informative. For instance, in some instances, a mutation that is present in both the cancer genome of the subject and the germline of the subject is informative for precision oncology when the mutation is a so-called ‘driver mutation,’ which contributes to the initiation and/or development of a cancer. However, in other instances, a mutation that is present in both the cancer genome of the subject and the germline of the subject is not informative for precision oncology, e.g., when the mutation is a so-called ‘passenger mutation,’ which does not contribute to the initiation and/or development of the cancer. Likewise, in some instances, a mutation that is present in the cancer genome of the subject but not the germline of the subject is informative for precision oncology, e.g., where the mutation is a driver mutation and/or the mutation facilitates a therapeutic approach, e.g., by differentiating cancer cells from normal cells in a therapeutically actionable way. However, in some instances, a mutation that is present in the cancer genome but not the germline of a subject is not informative for precision oncology, e.g., where the mutation is a passenger mutation and/or where the mutation fails to differentiate the cancer cell from a germline cell in a therapeutically actionable way.

As used herein, the term “reference allele” refers to the sequence of one or more nucleotides at a chromosomal locus that is either the predominant allele represented at that chromosomal locus within the population of the species (e.g., the “wild-type” sequence), or an allele that is predefined within a reference genome for the species.

As used herein, the term “variant allele” refers to a sequence of one or more nucleotides at a chromosomal locus that is either not the predominant allele represented at that chromosomal locus within the population of the species (e.g., not the “wild-type” sequence), or not an allele that is predefined within a reference genome for the species.

As used herein, the term “variant allele fraction,” “VAF,” “allelic fraction,” or “AF” refers to the number of times a variant or mutant allele was observed (e.g., a number of reads supporting a candidate variant allele) divided by the total number of times the position was sequenced (e.g., a total number of reads covering a candidate locus).

As used herein, the term “variant fragment count” refers to a quantification, e.g., a raw or normalized count, of the number of sequences representing unique cell-free DNA fragments encompassing the variant allele in a sequencing reaction. That is, a variant fragment count represents a count of sequence reads representing unique molecules in the liquid biological sample, after duplicate sequence reads in the raw sequencing data have been collapsed, e.g., through the use of UMI and bagging, etc. as described herein.

As used herein, the term “germline variants” refers to genetic variants inherited from maternal and paternal DNA. Germline variants may be determined through a matched tumor-normal calling pipeline.

As used herein, the term “somatic variants” refers to variants arising as a result of dysregulated cellular processes associated with neoplastic cells, e.g., a mutation. Somatic variants may be detected via subtraction from a matched normal sample.

As used herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

As used herein, the term “insertions and deletions” or “indels” refers to a variant resulting from the gain or loss of DNA base pairs within an analyzed region.

As used herein, the term “copy number variation” or “CNV” refers to the process by which large structural changes in a genome associated with tumor aneuploidy and other dysregulated repair systems are detected. These processes are used to detect large scale insertions or deletions of entire genomic regions. CNV is defined as structural insertions or deletions greater than a certain base pair (“bp”) in size, such as 500 bp.

As used herein, the term “gene fusion” refers to the product of large scale chromosomal aberrations resulting in the creation of a chimeric protein. These expressed products can be non-functional, or they can be highly over or underactive. This can cause deleterious effects in cancer such as hyper-proliferative or anti-apoptotic phenotypes.

As used herein, the term “loss of heterozygosity” refers to the loss of one copy of a segment (e.g., including part or all of one or more genes) of the genome of a diploid subject (e.g., a human) or loss of one copy of a sequence encoding a functional gene product in the genome of the diploid subject, in a tissue, e.g., a cancerous tissue, of the subject. As used herein, when referring to a metric representing loss of heterozygosity across the entire genome of the subject, loss of heterozygosity is caused by the loss of one copy of various segments in the genome of the subject. Loss of heterozygosity across the entire genome may be estimated without sequencing the entire genome of a subject, and such methods for such estimations based on gene panel targeting-based sequencing methodologies are described in the art. Accordingly, in some embodiments, a metric representing loss of heterozygosity across the entire genome of a tissue of a subject is represented as a single value, e.g., a percentage or fraction of the genome. In some cases a tumor is composed of various sub-clonal populations, each of which may have a different degree of loss of heterozygosity across their respective genomes. Accordingly, in some embodiments, loss of heterozygosity across the entire genome of a cancerous tissue refers to an average loss of heterozygosity across a heterogeneous tumor population. As used herein, when referring to a metric for loss of heterozygosity in a particular gene, e.g., a DNA repair protein such as a protein involved in the homologous DNA recombination pathway (e.g., BRCA1 or BRCA2), loss of heterozygosity refers to complete or partial loss of one copy of the gene encoding the protein in the genome of the tissue and/or a mutation in one copy of the gene that prevents translation of a full-length gene product, e.g., a frameshift or truncating (creating a premature stop codon in the gene) mutation in the gene of interest. In some cases a tumor is composed of various sub-clonal populations, each of which may have a different mutational status in a gene of interest. Accordingly, in some embodiments, loss of heterozygosity for a particular gene of interest is represented by an average value for loss of heterozygosity for the gene across all sequenced sub-clonal populations of the cancerous tissue. In other embodiments, loss of heterozygosity for a particular gene of interest is represented by a count of the number of unique incidences of loss of heterozygosity in the gene of interest across all sequenced sub-clonal populations of the cancerous tissue (e.g., the number of unique frame-shift and/or truncating mutations in the gene identified in the sequencing data).

As used herein, the term “microsatellites” refers to short, repeated sequences of DNA. The smallest nucleotide repeated unit of a microsatellite is referred to as the “repeated unit” or “repeat unit.” In some embodiments, the stability of a microsatellite locus is evaluated by comparing some metric of the distribution of the number of repeated units at a microsatellite locus to a reference number or distribution.

As used herein, the term “microsatellite instability” or “MSI” refers to a genetic hypermutability condition associated with various cancers that results from impaired DNA mismatch repair (MMR) in a subject. Among other phenotypes, MSI causes changes in the size of microsatellite loci, i.e., a change in the number of repeated units at microsatellite loci, during DNA replication. Accordingly, the size of microsatellite repeats is varied in MSI cancers as compared to the size of the corresponding microsatellite repeats in the germline of a cancer subject. The term “Microsatellite Instability-High” or “MSI-H” refers to a state of a cancer (e.g., a tumor) that has a significant MMR defect, resulting in microsatellite loci with significantly different lengths than the corresponding microsatellite loci in normal cells of the same individual. The term “Microsatellite Stable” or “MSS” refers to a state of a cancer (e.g., a tumor) without significant MMR defects, such that there is no significant difference between the lengths of the microsatellite loci in cancerous cells and the lengths of the corresponding microsatellite loci in normal (e.g., non-cancerous) cells in the same individual. The term “Microsatellite Equivocal” or “MSE” refers to a state of a cancer (e.g., a tumor) having an intermediate microsatellite length phenotype, that cannot be clearly classified as MSI-H or MSS based on statistical cutoffs used to define those two categories.

As used herein, the term “gene product” refers to an RNA (e.g., mRNA or miRNA) or protein molecule transcribed or translated from a particular genomic locus, e.g., a particular gene. The genomic locus can be identified using a gene name, a chromosomal location, or any other genetic mapping metric.

As used herein, the term “ratio” refers to any comparison of a first metric X, or a first mathematical transformation thereof X′ (e.g., measurement of a number of units of a genomic sequence in a first one or more biological samples or a first mathematical transformation thereof) to another metric Y or a second mathematical transformation thereof Y′ (e.g., the number of units of a respective genomic sequence in a second one or more biological samples or a second mathematical transformation thereof) expressed as X/Y, Y/X, logN(X/Y), logN(Y/X), X′/Y, Y/X′, logN(X′/Y), or logN(Y/X′), X/Y′, Y′/X, logN(X/Y′), logN(Y′/X), X′/Y′, Y′/X′, logN(X′/Y′), or logN(Y′/X′), where N is any real number greater than 1 and where example mathematical transformations of X and Y include, but are not limited to. raising X or Y to a power Z, multiplying X or Y by a constant Q, where Z and Q are any real numbers, and/or taking an M based logarithm of X and/or Y, where M is a real number greater than 1. In one non-limiting example, X is transformed to X′ prior to ratio calculation by raising X by the power of two (X²) and Y is transformed to Y′ prior to ratio calculation by raising Y by the power of 3.2 (Y^{3.2}) and the ratio of X and Y is computed as log₂(X′/Y′).

As used herein, the terms “expression level,” “abundance level,” or simply “abundance” refers to an amount of a gene product, (an RNA species, e.g., mRNA or miRNA, or protein molecule) transcribed or translated by a cell, or an average amount of a gene product transcribed or translated across multiple cells. When referring to mRNA or protein expression, the term generally refers to the amount of any RNA or protein species corresponding to a particular genomic locus, e.g., a particular gene. However, in some embodiments, an expression level can refer to the amount of a particular isoform of an mRNA or protein corresponding to a particular gene that gives rise to multiple mRNA or protein isoforms. The genomic locus can be identified using a gene name, a chromosomal location, or any other genetic mapping metric.

As used herein, the term “relative abundance” refers to a ratio of a first amount of a compound measured in a sample, e.g., a gene product (an RNA species, e.g., mRNA or miRNA, or protein molecule) or nucleic acid fragments having a particular characteristic (e.g., aligning to a particular locus or encompassing a particular allele), to a second amount of a compound measured in a second sample. In some embodiments, relative abundance refers to a ratio of an amount of species of a compound to a total amount of the compound in the same sample. For instance, a ratio of the amount of mRNA transcripts encoding a particular gene in a sample (e.g., aligning to a particular region of the exome) to the total amount of mRNA transcripts in the sample. In other embodiments, relative abundance refers to a ratio of an amount of a compound or species of a compound in a first sample to an amount of the compound of the species of the compound in a second sample. For instance, a ratio of a normalized amount of mRNA transcripts encoding a particular gene in a first sample to a normalized amount of mRNA transcripts encoding the particular gene in a second and/or reference sample.

As used herein, the terms “sequencing,” “sequence determination,” and the like refer to any biochemical processes that may be used to determine the order of biological macromolecules such as nucleic acids or proteins. For example, sequencing data can include all or a portion of the nucleotide bases in a nucleic acid molecule such as an mRNA transcript or a genomic locus.

As used herein, the term “nucleic acid sequence” refers to a recordation of a series of nucleotides present in a subject's RNA (e.g., mRNA) or DNA (e.g., genomic DNA) as determined by sequencing of nucleic acids from the subject.

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore® sequencing, for example, can provide sequence reads that can vary in size from tens to hundreds to thousands of base pairs. Illumina® parallel sequencing, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “read segment” refers to any form of nucleotide sequence read including the raw sequence reads obtained directly from a nucleic acid sequencing technique or from a sequence derived therefrom, e.g., an aligned sequence read, a collapsed sequence read, or a stitched sequence read.

As used herein, the term “read count” refers to the total number of nucleic acid reads generated, which may or may not be equivalent to the number of nucleic acid molecules generated, during a nucleic acid sequencing reaction.

As used herein, the term “read-depth,” “sequencing depth,” or “depth” can refer to a total number of unique nucleic acid fragments encompassing a particular locus or region of the genome of a subject that are sequenced in a particular sequencing reaction. Sequencing depth can be expressed as “Yx”, e.g., 50×, 100×, etc., where “Y” refers to the number of unique nucleic acid fragments encompassing a particular locus that are sequenced in a sequencing reaction. In such a case, Y is necessarily an integer, because it represents the actual sequencing depth for a particular locus. Alternatively, read-depth, sequencing depth, or depth can refer to a measure of central tendency (e.g., a mean or mode) of the number of unique nucleic acid fragments that encompass one of a plurality of loci or regions of the genome of a subject that are sequenced in a particular sequencing reaction. For example, in some embodiments, sequencing depth refers to the average depth of every locus across an arm of a chromosome, a targeted sequencing panel, an exome, or an entire genome. In such case, Y may be expressed as a fraction or a decimal, because it refers to an average coverage across a plurality of loci. When a mean depth is recited, the actual depth for any particular locus may be different than the overall recited depth. Metrics can be determined that provide a range of sequencing depths in which a defined percentage of the total number of loci fall. For instance, a range of sequencing depths within which 90% or 95%, or 99% of the loci fall. As understood by the skilled artisan, different sequencing technologies provide different sequencing depths. For instance, low-pass whole genome sequencing can refer to technologies that provide a sequencing depth of less than 5×, less than 4×, less than 3×, or less than 2×, e.g., from about 0.5× to about 3×.

As used herein, the term “sequencing breadth” refers to what fraction of a particular reference exome (e.g., human reference exome), a particular reference genome (e.g., human reference genome), or part of the exome or genome has been analyzed. Sequencing breadth can be expressed as a fraction, a decimal, or a percentage, and is generally calculated as (the number of loci analyzed/the total number of loci in a reference exome or reference genome). The denominator of the fraction can be a repeat-masked genome, and thus 100% can correspond to all of the reference genome minus the masked parts. A repeat-masked exome or genome can refer to an exome or genome in which sequence repeats are masked (e.g., sequence reads align to unmasked portions of the exome or genome). In some embodiments, any part of an exome or genome can be masked and, thus, sequencing breadth can be evaluated for any desired portion of a reference exome or genome. In some embodiments, “broad sequencing” refers to sequencing/analysis of at least 0.1% of an exome or genome.

As used herein, the term “sequencing probe” refers to a molecule that binds to a nucleic acid with affinity that is based on the expected nucleotide sequence of the RNA or DNA present at that locus.

As used herein, the term “targeted panel” or “targeted gene panel” refers to a combination of probes for sequencing (e.g., by next-generation sequencing) nucleic acids present in a biological sample from a subject (e.g., a tumor sample, liquid biopsy sample, germline tissue sample, white blood cell sample, or tumor or tissue organoid sample), selected to map to one or more loci of interest on one or more chromosomes. An example set of loci/genes useful for precision oncology, e.g., via solid or liquid biopsy assay, that can be analyzed using a targeted panel is described in Table 1. In some embodiments, in addition to loci that are informative for precision oncology, a targeted panel includes one or more probes for sequencing one or more of a loci associated with a different medical condition, a loci used for internal control purposes, or a loci from a pathogenic organism (e.g., an oncogenic pathogen).

As used herein, the term “reference construct” refers to a plurality of reference nucleic acid sequences, or a graphical representation, hashed representation, or similar representation thereof, corresponding to all or a portion of an exome, genome, transcriptome, etc., for a species. In some embodiments, a reference construct refers to a plurality of reference nucleic acid sequences, or representations thereof, corresponding to a panel of probes used to enrich targeted nucleic acids prior to a sequencing reaction. In other embodiments, a reference construct refers to a reference exome, reference transcriptome, or reference genome, or a representation thereof, for a species.

As used herein, the term “reference exome” refers to any sequenced or otherwise characterized exome, whether partial or complete, of any tissue from any organism or pathogen that may be used to reference identified sequences from a subject. Typically, a reference exome will be derived from a subject of the same species as the subject whose sequences are being evaluated. Example reference exomes used for human subjects as well as many other organisms are provided in the on-line genome browser hosted by the National Center for Biotechnology Information (“NCBI”). An “exome” refers to the complete transcriptional profile of an organism or pathogen, expressed in nucleic acid sequences. As used herein, a reference sequence or reference exome often is an assembled or partially assembled exomic sequence from an individual or multiple individuals. In some embodiments, a reference exome is an assembled or partially assembled exomic sequence from one or more human individuals. The reference exome can be viewed as a representative example of a species' set of expressed genes. In some embodiments, a reference exome comprises sequences assigned to chromosomes.

As used herein, the term “reference genome” refers to any sequenced or otherwise characterized genome, whether partial or complete, of any organism or pathogen that may be used to reference identified sequences from a subject. Typically, a reference genome will be derived from a subject of the same species as the subject whose sequences are being evaluated. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or pathogen, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38). For a haploid genome, there can be only one nucleotide at each locus. For a diploid genome, heterozygous loci can be identified; each heterozygous locus can have two alleles, where either allele can allow a match for alignment to the locus.

As used herein, the term “bioinformatics pipeline” refers to a series of processing stages used to determine characteristics of a subject's genome or exome based on sequencing data of the subject's genome or exome. A bioinformatics pipeline may be used to determine characteristics of a germline genome or exome of a subject and/or a cancer genome or exome of a subject. In some embodiments, the pipeline extracts information related to genomic alterations in the cancer genome of a subject, which is useful for guiding clinical decisions for precision oncology, from sequencing results of a biological sample, e.g., a tumor sample, liquid biopsy sample, reference normal sample, etc., from the subject. Certain processing stages in a bioinformatics may be ‘connected,’ meaning that the results of a first respective processing stage is informative and/or essential for execution of a second, downstream processing stage. For instance, in some embodiments, a bioinformatics pipeline includes a first respective processing stage for identifying genomic alterations that are unique to the cancer genome of a subject and a second respective processing stage that uses the quantity and/or identity of the identified genomic alterations to determine a metric that is informative for precision oncology, e.g., a tumor mutational burden. In some embodiments, the bioinformatics pipeline includes a reporting stage that generates a report of relevant and/or actionable information identified by upstream stages of the pipeline, which may or may not further include recommendations for aiding clinical therapy decisions.

As used herein, the term “limit of detection” or “LOD” refers to the minimal quantity of a feature that can be identified with a particular level of confidence. Accordingly, level of detection can be used to describe an amount of a substance that must be present in order for a particular assay to reliably detect the substance. A level of detection can also be used to describe a level of support needed for an algorithm to reliably identify a genomic alteration based on sequencing data. For example, a minimal number of unique sequence reads to support identification of a sequence variant such as a SNV.

As used herein, the term “BAM File” or “Binary file containing Alignment Maps” refers to a file storing sequencing data aligned to a reference sequence (e.g., a reference genome or exome). In some embodiments, a BAM file is a compressed binary version of a SAM (Sequence Alignment Map) file that includes, for each of a plurality of unique sequence reads, an identifier for the sequence read, information about the nucleotide sequence, information about the alignment of the sequence to a reference sequence, and optionally metrics relating to the quality of the sequence read and/or the quality of the sequence alignment. While BAM files generally relate to files having a particular format, for simplicity they are used herein to simply refer to a file, of any format, containing information about a sequence alignment, unless specifically stated otherwise.

As used herein, the term “measure of central tendency” refers to a central or representative value for a distribution of values. Non-limiting examples of measures of central tendency include an arithmetic mean, weighted mean, midrange, midhinge, trimean, geometric mean, geometric median, Winsorized mean, median, and mode of the distribution of values.

As used herein, the term “Positive Predictive Value” or “PPV” means the likelihood that a variant is properly called given that a variant has been called by an assay. PPV can be expressed as (number of true positives)/(number of false positives+number of true positives).

As used herein, the term “assay” refers to a technique for determining a property of a substance, e.g., a nucleic acid, a protein, a cell, a tissue, or an organ. An assay (e.g., a first assay or a second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art can be used to detect any of the properties of nucleic acids mentioned herein. Properties of a nucleic acids can include a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid fragments). An assay or method can have a particular sensitivity and/or specificity, and their relative usefulness as a diagnostic tool can be measured using ROC-AUC statistics.

As used herein, the term “classification” can refer to any number(s) or other characters(s) that are associated with a particular property of a sample. For example, in some embodiments, the term “classification” can refer to a type of cancer in a subject, a stage of cancer in a subject, a prognosis for a cancer in a subject, a tumor load, a presence of tumor metastasis in a subject, and the like. The classification can be binary (e.g., positive or negative) or have more levels of classification (e.g., a scale from 1 to 10 or 0 to 1). The terms “cutoff” and “threshold” can refer to predetermined numbers used in an operation. For example, a cutoff size can refer to a size above which fragments are excluded. A threshold value can be a value above or below which a particular classification applies. Either of these terms can be used in either of these contexts.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, an “actionable genomic alteration” or “actionable variant” refers to a genomic alteration (e.g., a SNV, MNV, indel, rearrangement, gene fusion, copy number variation, or ploidy variation), or value of another cancer metric derived from nucleic acid sequencing data (e.g., a tumor mutational burden, MSI status, or tumor fraction), that is known or believed to be associated with a therapeutic course of action that is more likely to produce a positive effect in a patient that has the actionable variant than in a similarly situated patient that does not have the actionable variant. For instance, administration of EGFR inhibitors (e.g., afatinib, erlotinib, gefitinib) is more effective for treating non-small cell lung cancer in patients with an EGFR mutation in exons 19/21 than for treating non-small cell lung cancer in patients that do not have an EGFR mutations in exons 19/21. Accordingly, an EGFR mutation in exon 19/21 is an actionable variant. In some instances, an actionable variant is only associated with an improved treatment outcome in one or a group of specific cancer types. In other instances, an actionable variant is associated with an improved treatment outcome in substantially all cancer types.

As used herein, “gene isoforms” or “isoforms” refer to mRNA molecules, transcribed from the same genomic locus, that include different combinations of partial and/or complete exons. Different gene isoforms can be formed, for example, by use of alternative transcriptional start sites, early transcriptional termination, and alternative mRNA splicing (e.g., through cryptic splice sites).

As used herein, an “actionable mRNA isoform,” “actionable mRNA isoform pattern,” and “actionable mRNA splicing pattern” interchangeably refer to a particular mRNA splicing status (e.g., the presence of a particular isoform, the absence of a particular isoform, the presence of a particular pattern of isoforms for one or more genes, a relative abundance of one or more isoforms, etc.) that is known or believed to be associated with a therapeutic course of action that is more likely to produce a positive effect in a patient that has the particular mRNA splicing status than in a similarly situated patient that does not have the particular mRNA splicing status.

As used herein, a “variant of uncertain significance” or “VUS” refers to a genomic alteration (e.g., a SNV, MNV, indel, rearrangement, copy number variation, or ploidy variation), or value of another cancer metric derived from nucleic acid sequencing data (e.g., a tumor mutational burden, MSI status, or tumor fraction), whose impact on disease development/progression is unknown.

As used herein, a “benign variant” or “likely benign variant” refers to a genomic alteration (e.g., a SNV, MNV, indel, rearrangement, copy number variation, or ploidy variation), or value of another cancer metric derived from nucleic acid sequencing data (e.g., a tumor mutational burden, MSI status, or tumor fraction), that is known or believed to not contribute to disease development/progression.

As used herein, a “pathogenic variant” or “likely pathogenic variant” refers to a genomic alteration (e.g., a SNV, MNV, indel, rearrangement, copy number variation, or ploidy variation), or value of another cancer metric derived from nucleic acid sequencing data (e.g., a tumor mutational burden, MSI status, or tumor fraction), that is known or believed to contribute to disease development/progression.

As used herein, an “effective amount” or “therapeutically effective amount” is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the therapeutic agent being administered.

As used herein, the terms “classifier” and “model” are used interchangeably and include any parametric, semiparametric, or non-perimetric model, including statistical inference models and machine learning models.

In some embodiments, a model is an unsupervised learning algorithm. One example of an unsupervised learning algorithm is cluster analysis.

In some embodiments, a model is a supervised machine learning algorithm. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a classifier is a multinomial classifier algorithm. In some embodiments, a classifier is a 2-stage stochastic gradient descent (SGD) model. In some embodiments, a classifier is a deep neural network (e.g., a deep-and-wide sample-level classifier).

Neural networks. In some embodiments, the model is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in the present disclosure. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used in the present disclosure in accordance with the present disclosure.

For instance, a deep neural network classifier comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network classifier. In some embodiments, at least 100 parameters, at least 1000 parameters, at least 2000 parameters or at least 5000 parameters are associated with the deep neural network classifier. As such, deep neural network classifiers require a computer to be used because they cannot be mentally solved. In other words, given an input to the classifier, the classifier output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as classifiers are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as classifiers are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as classifiers are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the model is a support vector machine (SVM) algorithm. SVM algorithms suitable for use as classifiers are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM classifier requires a computer to calculate because it cannot be mentally solved.

Naïve Bayes algorithms. In some embodiments, the model is a Naive Bayes algorithm. Naïve Bayes classifiers suitable for use as classifiers are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes classifier is any classifier in a family of “probabilistic classifiers” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a model is a nearest neighbor algorithm. Nearest neighbor models can be memory-based and include no classifier to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. Here, the distance to these neighbors is a function of the abundance values of the discriminating gene set. In some embodiments, Euclidean distance in feature space is used to determine distance as d₍ᵢ₎=∥x₍ᵢ₎−x₍₀₎∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning model in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor classifier is such that a computer is used to solve the classifier for a given input because it cannot be mentally performed.

Random forest, decision tree, and boosted tree algorithms. In some embodiments, the model is a decision tree. Decision trees suitable for use as classifiers are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests-Random Features,” Technical Report 567, Statistics Department, U.C. Berkeley, September 1999, which is hereby incorporated by reference in its entirety. In some embodiments, the decision tree classifier includes at least 10, at least 20, at least 50, or at least 100 parameters (e.g., weights and/or decisions) and requires a computer to calculate because it cannot be mentally solved.

Regression. In some embodiments, the model uses a regression algorithm. A regression algorithm can be any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the classifier. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the classifier makes use of a regression model disclosed in Hastie et al., 2001, The Elements of Statistical Learning, Springer-Verlag, New York. In some embodiments, the logistic regression classifier includes at least 10, at least 20, at least 50, at least 100, or at least 1000 parameters (e.g., weights) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the classifier (linear classifier) in some embodiments of the present disclosure.

Mixture model and Hidden Markov model. In some embodiments, the model is a mixture model, such as that described in McLachlan et al., Bioinformatics 18(3):413-422, 2002. In some embodiments, in particular, those embodiments including a temporal component, the classifier is a hidden Markov model such as described by Schliep et al., 2003, Bioinformatics 19(1):i255-i263.

Clustering. In some embodiments, the model is an unsupervised clustering model. In some embodiments, the classifier is a supervised clustering model. Clustering algorithms suitable for use as classifiers are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of classifiers and boosting. In some embodiments, an ensemble (two or more) of models is used. In some embodiments, a boosting technique such as AdaBoost is used in conjunction with many other types of learning algorithms to improve the performance of the classifier. In this approach, the output of any of the classifiers disclosed herein, or their equivalents, is combined into a weighted sum that represents the final output of the boosted classifier. In some embodiments, the plurality of outputs from the classifiers is combined using any measure of central tendency known in the art, including but not limited to a mean, median, mode, a weighted mean, weighted median, weighted mode, etc. In some embodiments, the plurality of outputs is combined using a voting method. In some embodiments, a respective classifier in the ensemble of classifiers is weighted or unweighted.

Generally, training a model (e.g., logistic regression model, a neural network, and/or another suitable model) includes updating the plurality of parameters for the respective classifier through backpropagation (e.g., gradient descent). First, a forward propagation is performed, in which input data is accepted into the untrained or partially untrained model, and an output is calculated based on the selected activation function and an initial set of parameters (e.g., weights). A backward pass can then be performed by calculating an error gradient for each respective parameter, where the error for each parameter is determined by calculating a loss (e.g., error) based on the output (e.g., the predicted value) and the input data (e.g., the expected value or true labels).

Training is performed against a training dataset that includes features and labels for each of a plurality of training samples. In some embodiments, the training data set is specific for a particular characteristic, e.g., a type of disease or disorder (e.g., a type of cancer) or a personal characteristic, e.g., an age, gender, ethnicity, etc., of the training subjects, that is the model is trained for use on a specific population. In some embodiments, one of more of a type of disease or disorder and/or personal characteristic is used as a feature of the model itself, that is the model accounts for these variables when providing an output.

Parameters can then be updated by adjusting the value based on the calculated loss metered by a predetermined learning rate hyperparameter that dictates the degree or severity to which parameters are updated (e.g., small adjustments versus large adjustments), thereby training the untrained or partially untrained model.

For example, in some general embodiments of machine learning, backpropagation is a method of training an untrained or partially untrained model comprising a plurality of parameters (e.g., embeddings). The output of an untrained or partially untrained model (e.g., a measure of the complexity of the sequencing data, the identification of a particular mRNA species, the elucidation of an mRNA isoform pattern, etc.) can be generated using a set of arbitrarily selected initial parameters. The output is then compared with the original input (e.g., a known measure of the complexity of the sequencing data, a ground truth for the presence of a particular mRNA species, a ground truth for an mRNA isoform pattern, etc., of the respective training subject) by evaluating an error function to compute an error (e.g., using a loss function). The parameters can then be updated such that the error is minimized (e.g., according to the loss function). In some embodiments, any one of a variety of backpropagation algorithms and/or methods are used to update the plurality of parameters.

In some embodiments, the error is computed using an error function (e.g., a loss function). In some embodiments, the loss function is mean square error, quadratic loss, mean absolute error, mean bias error, hinge, multi-class support vector machine, and/or cross-entropy. In some embodiments, training the untrained or partially untrained model comprises computing an error in accordance with a gradient descent algorithm and/or a minimization function.

In some embodiments, the error function is used to update one or more parameters in an untrained or partially untrained model by adjusting the value of the one or more parameters by an amount proportional to the calculated loss, thereby training the model. In some embodiments, the amount by which the parameters are adjusted is metered by a predetermined learning rate that dictates the degree or severity to which parameters are updated (e.g., smaller or larger adjustments). In some embodiments, the learning rate is a hyperparameter that can be selected by a practitioner.

In some embodiments, training the untrained or partially untrained model forms a trained classifier following a first evaluation of an error function. In some such embodiments, training the untrained or partially untrained model forms a trained classifier following a first updating of one or more parameters based on a first evaluation of an error function. In some alternative embodiments, training the untrained or partially untrained model forms a trained classifier following at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million evaluations of an error function. In some such embodiments, training the untrained or partially untrained model forms a trained classifier following at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million updatings of one or more parameters based on the at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million evaluations of an error function.

In some embodiments, training the untrained or partially untrained model forms a trained classifier when the model satisfies a minimum performance requirement. For example, in some embodiments, training the untrained or partially untrained model forms a trained classifier when the error calculated for the trained classifier, following an evaluation of an error function across one or more training datasets for a respective one or more training subjects, satisfies an error threshold. In some embodiments, the error calculated by the error function across one or more training datasets for a respective one or more training subjects satisfies an error threshold when the error is less than 20 percent, less than 18 percent, less than 15 percent, less than 10 percent, less than 5 percent, or less than 3 percent.

In some embodiments, the minimum performance requirement is satisfied based on a validation training. In some embodiments, validation training is performed through K-fold cross-validation.

In some embodiments, classifier training is performed on a plurality of machines (e.g., computers and/or systems). In some embodiments, using the classifier to variant allele at a genomic position in a test subject as somatic or germline is performed on a plurality of machines (e.g., computers and/or systems).

In some embodiments, model training further comprises fixing (e.g., freezing) one or more parameters in the plurality of parameters, thereby obtaining a corresponding trained classifier that can be used to perform determination and/or classification (e.g., of a measure of the complexity of the sequencing data, the identification of a particular mRNA species, the elucidation of an mRNA isoform pattern, etc.).

Any other model parameters and architectures suitable for training are contemplated, as will be apparent to one skilled in the art.

In some embodiments, features that are used in the training of the model include gene fusion information. Clinical information about a particular fusion informs predictions of the interactions within the progression of cancer and phenotype. Raw Sequence level information can help to track the break points, possible mode of action (DNA level fusion, RNA splice, RNA), and give population level data about the fusions. The sequence level data could also be used in predictive level for weak points, prediction of possible novel fusions that might be seen and predict the likelihood of a fusion if a novel fusion is observed. It would be the basis of how the scoring would work in a population estimate of sensitivity. For example, informing what the fusion is, how often the fusion is observed, whether there are similar fusions, and/or whether there is a common side of the fusion that is fused to an uncommon region.

In some embodiments, features that are used in training of the model include RNA-seq transcriptome data. In some embodiments, a model is trained against examples of normal and diseased transcriptome data to provide perspective on the differences seen in the transcriptome of diseased and non-diseased samples, as well as to provide insight in the types of artifacts typically observed in both types of samples and to consider different biological factors, including genetic origin, tissue of origin, and gene function. This sets a baseline for the boundary elements expected for a particular type of sample, and optionally for a particular ethnic origin or molecular profile. In some embodiments, this can also be done in conjunction with methylation data to establish the related methylation profile and methylation status, e.g., in a database. The methylation profile of a genome influences the expression profile and, thereby, the boundary element profile expected for a particular tissue. In some embodiments, methylation status can be tracked over time and corresponding shifts in the boundary element profile for a tissue can be correlated to particular methylation patterns.

In some embodiments, features that are used in training of the model include gene sequences and/or genomic sequencing data. This data benchmarks RNA sequencing data by further evidencing genomic alterations reflected in a transcriptome sequencing result. This would also allow identification of RNA alterations that are not reflected in the underlying genome. In this fashion, transcription elements that are part of a novel fusion may be identified as drivers of biological differences that can further inform clinical treatment of the subject. The may enable prediction of the extent to which a particular fusion is expressed, based on the underlying DNA sequencing data.

In some embodiments, features that are used in the training of the model include fusion breakpoint coverage, as well as direct and indirect reads evidencing a breakpoint. Information on fusion breakpoints and exon boundaries facilitate better understanding of the resulting phenotype of the disease and its characteristics. In this fashion, observed boundaries can be associated with specific disease states, ethic origin, tissue of origin, and other molecular phenotypes, in the model.

In some embodiments, features that are used in the training of the model include a tissue of origin. Tissue of origin can influence gene expression and, thus, expected RNA boundary profiles. It can also be leveraged to help identify a tissue of origin when unknown at the time of biopsy. Establishing correlations between tissue of origin and RNA boundary profiles can further be integrated with methylation data to better characterize a sample.

In some embodiments, features that are used in the training of the model include a known prevalence for a particular mRNA species, e.g., a particular gene fusion and/or mRNA variant isoform. This information assists general profile building and testing of the Machine learning algorithm. The better established data and profiles are for known prevalence assists in weighting and refining model parameters.

In some embodiments, features that are used in the training of the model include coverage, in a sequencing reaction, across genes, exons, and/or boundary elements. These information can be combined with specific RNA boundary profiles to better define a model for sample complexity and/or genetic status.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

Reference will now be made in detail to embodiments, examples of which are illustrated in the accompanying drawings. In the following detailed description, numerous specific details are set forth in order to provide a thorough understanding of the present disclosure, including example systems, methods, techniques, instruction sequences, and computing machine program products that embody illustrative implementations. However, the illustrative discussions below are not intended to be exhaustive or to limit the implementations to the precise forms disclosed. Many modifications and variations are possible in view of the above teachings. The features described herein are not limited by the illustrated ordering of acts or events, as some acts can occur in different orders and/or concurrently with other acts or events.

Example System Embodiments

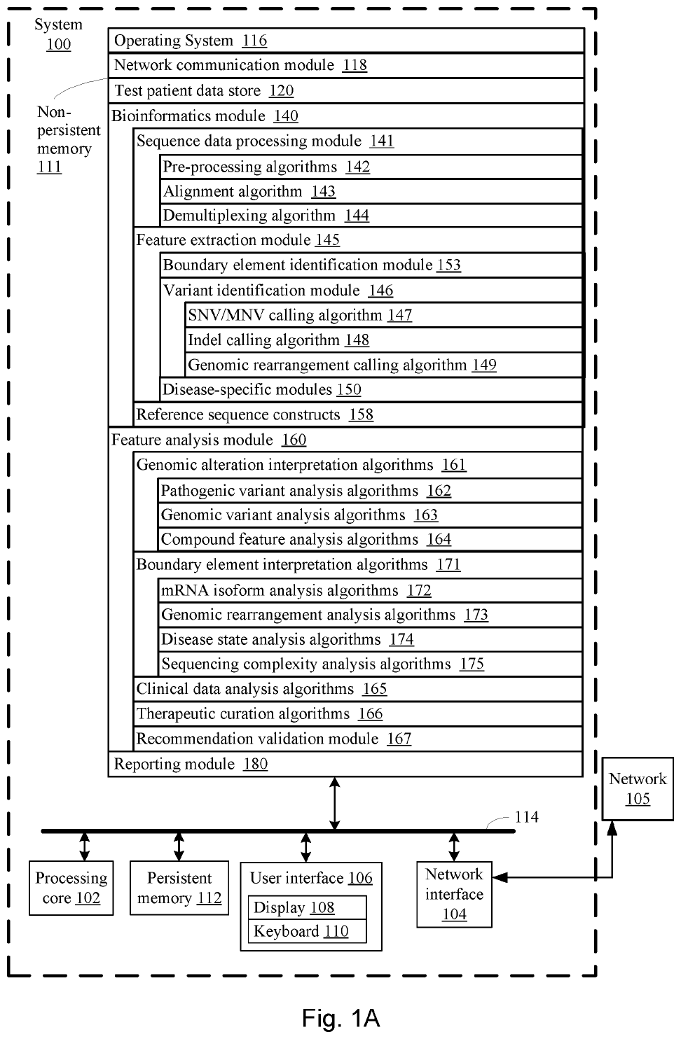

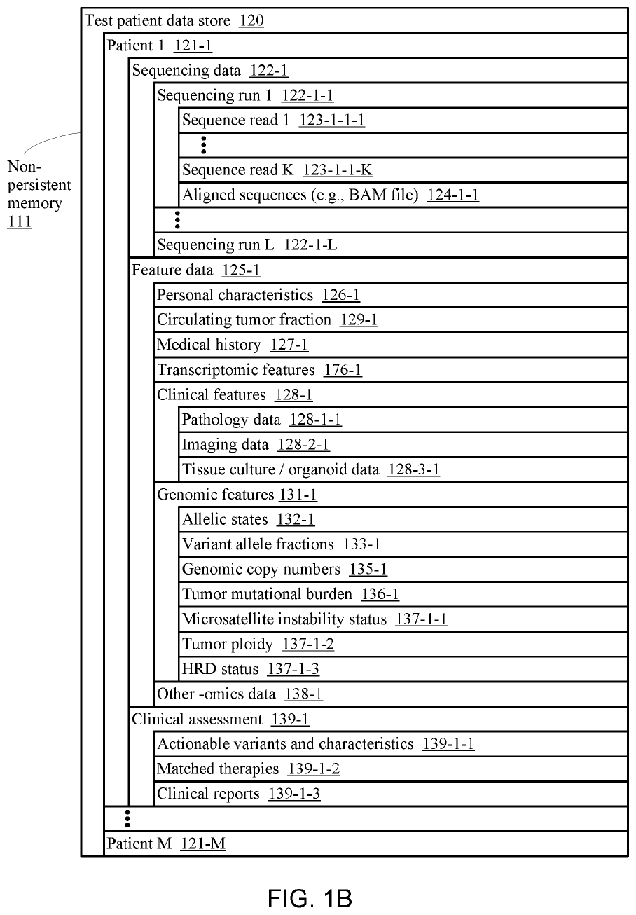

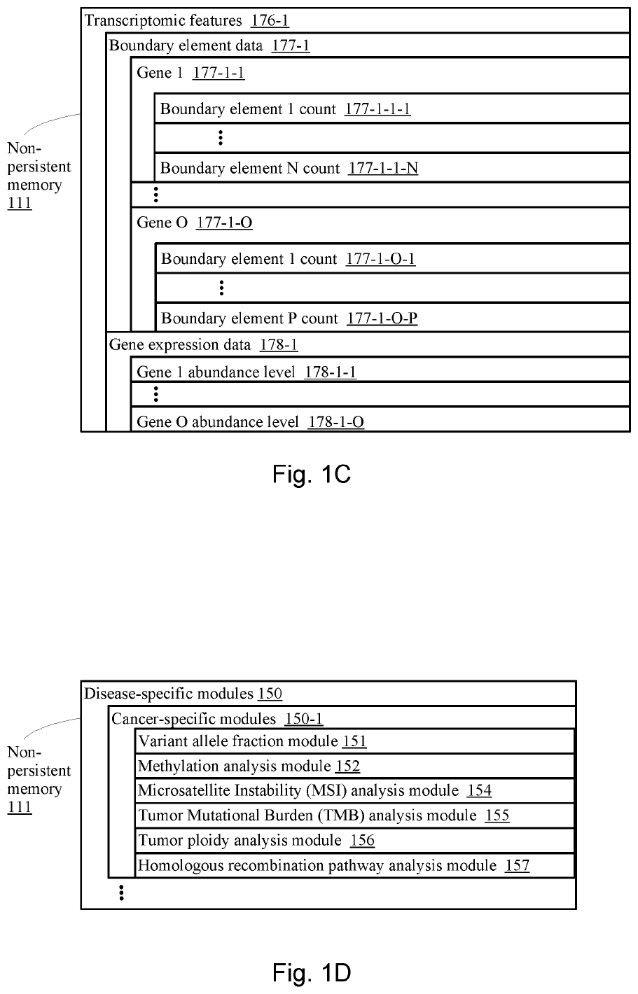

Now that an overview of some aspects of the present disclosure and some definitions used in the present disclosure have been provided, details of an exemplary system for providing clinical support for personalized therapy for various diseases and disorders (e.g., cardiovascular conditions, neurological conditions, cancers, etc.) are now described in conjunction with FIGS. 1A, 1B, 1C, and 1D. FIGS. 1A, 1B, 1C, and 1D collectively illustrate the topology of an example system for providing clinical support for personalized therapy, in accordance with some embodiments of the present disclosure. Advantageously, the example system illustrated in FIGS. 1A, 1B, 1C, and 1D improves upon conventional methods for providing clinical support for personalized therapy by improving determination of a genetic status of a subject and/or by improving evaluation of the nucleic acid complexity of a nucleic acid sequencing reaction, through various forms of mRNA boundary analysis.

FIG. 1A is a block diagram illustrating a system in accordance with some implementations. The device 100 in some implementations includes one or more processing units CPU(s) 102 (also referred to as processors), one or more network interfaces 104, a user interface 106, e.g., including a display 108 and/or an input 110 (e.g., a mouse, touchpad, keyboard, etc.), a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an operating system 116, which includes procedures for handling various basic system services and for performing hardware dependent tasks;
a network communication module (or instructions) 118 for connecting the system 100 with other devices and/or a communication network 105;
a test patient data store 120 for storing one or more collections of features from patients (e.g., subjects);
a bioinformatics module 140 for processing sequencing data and extracting features from sequencing data, e.g., from liquid biopsy sequencing assays;
a feature analysis module 160 for evaluating patient features, e.g., genomic alterations, compound genomic features, and clinical features; and
a reporting module 180 for generating and transmitting reports that provide clinical support for personalized cancer therapy.

Although FIGS. 1A, 1B, 1C, and 1D depict various components of a “system 100,” the figures are intended more as a functional description of the various features that may be present in computer systems than as a structural schematic of the implementations described herein. In practice, items shown separately could be combined and some items could be separated. Moreover, although FIG. 1 depicts certain data and modules in non-persistent memory 111, some or all of these data and modules may be in persistent memory 112. For example, in various implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations.

In some implementations, the non-persistent memory 111 optionally stores a subset of the modules and data structures identified above. Furthermore, in some embodiments, the memory stores additional modules and data structures not described above. In some embodiments, one or more of the above-identified elements is stored in a computer system, other than that of system 100, that is addressable by system 100 so that system 100 may retrieve all or a portion of such data when needed.

For purposes of illustration in FIG. 1A, system 100 is represented as a single computer that includes all of the functionality for providing clinical support for personalized cancer therapy. However, while a single machine is illustrated, the term “system” shall also be taken to include any collection of machines that individually or jointly execute a set (or multiple sets) of instructions to perform any one or more of the methodologies discussed herein.

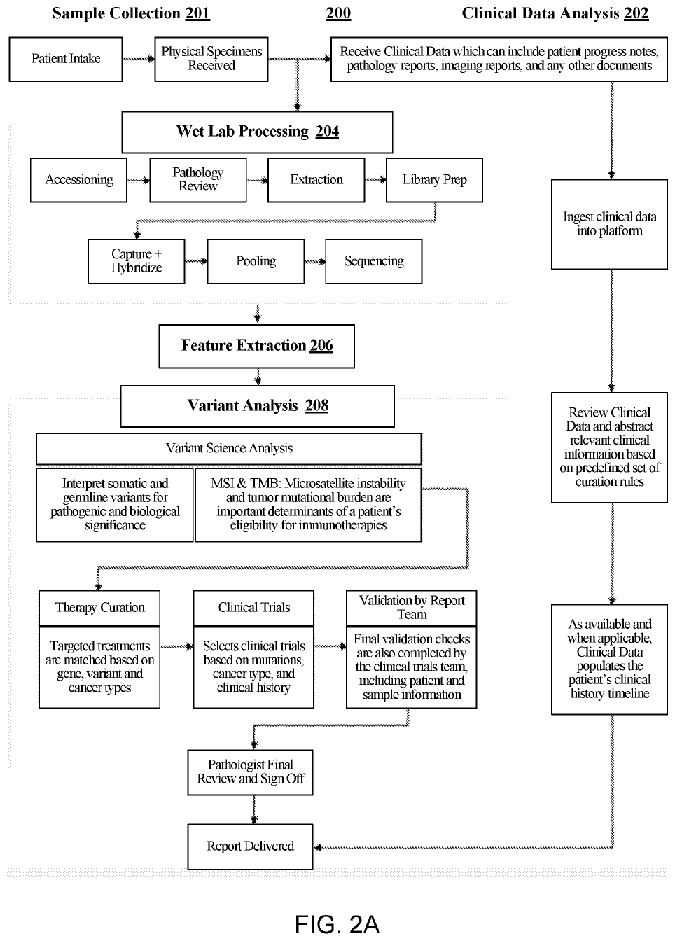

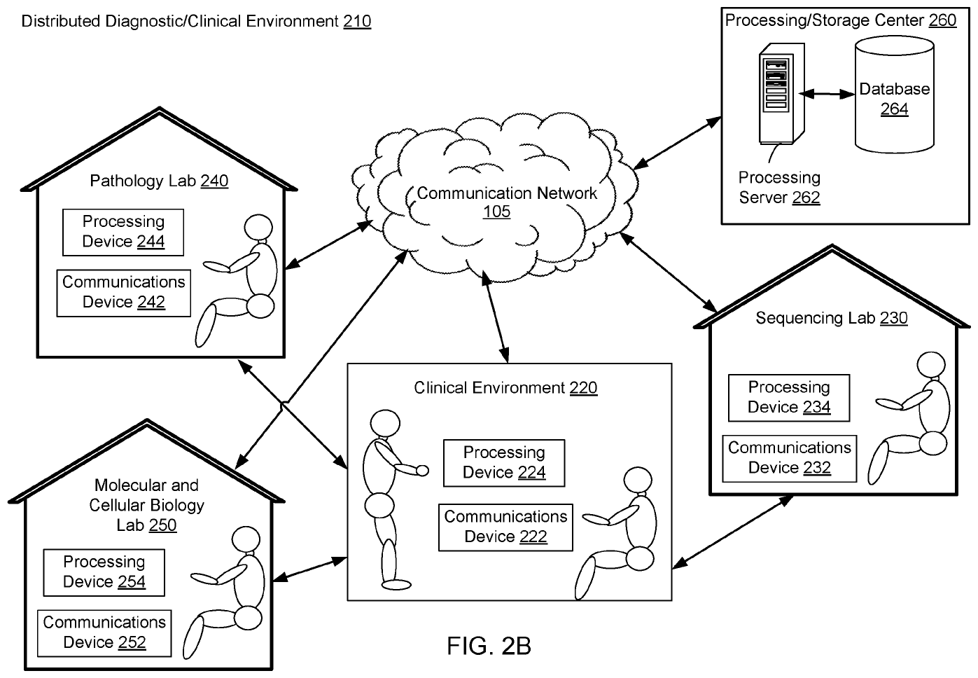

For example, in some embodiments, system 100 includes one or more computers. In some embodiments, the functionality for providing clinical support for personalized therapy is spread across any number of networked computers and/or resides on each of several networked computers and/or is hosted on one or more virtual machines at a remote location accessible across the communications network 105. For example, different portions of the various modules and data stores illustrated in FIGS. 1A, 1B, 1C, and 1D can be stored and/or executed on the various instances of a processing device and/or processing server/database in the distributed diagnostic environment 210 illustrated in FIG. 2B (e.g., processing devices 224, 234, 244, and 254, processing server 262, and database 264).

The system may operate in the capacity of a server or a client machine in a client-server network environment, as a peer machine in a peer-to-peer (or distributed) network environment, or as a server or a client machine in a cloud computing infrastructure or environment. The system may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine.

In another implementation, the system comprises a virtual machine that includes a module for executing instructions for performing any one or more of the methodologies disclosed herein. In computing, a virtual machine (VM) is an emulation of a computer system that is based on computer architectures and provides functionality of a physical computer. Some such implementations may involve specialized hardware, software, or a combination of hardware and software.

One of skill in the art will appreciate that any of a wide array of different computer topologies are used for the application and all such topologies are within the scope of the present disclosure.

Test Patient Data Store (120)

Referring to FIG. 1B, in some embodiments, the system (e.g., system 100) includes a patient data store 120 that stores data for patients 121-1 to 121-M including one or more sequencing data 122, feature data 125, and clinical assessments 139. These data are used and/or generated by the various processes stored in the bioinformatics module 140 and feature analysis module 160 of system 100, to ultimately generate a report providing clinical support for personalized therapy of a patient. While the feature scope of patient data 121 across all patients may be informationally dense, an individual patient's feature set may be sparsely populated across the entirety of the collective feature scope of all features across all patients. That is to say, the data stored for one patient may include a different set of features that the data stored for another patient. Further, while illustrated as a single data construct in FIG. 1B, different sets of patient data may be stored in different databases or modules spread across one or more system memories.

In some embodiments, sequencing data 122 from one or more sequencing reactions 122-i, including a plurality of sequence reads 123-1 to 123-K, is stored in the test patient data store 120. The data store may include different sets of sequencing data from a single subject, corresponding to different samples from the patient, e.g., salivary samples, blood samples, solid tissue samples, tumor samples, and/or to samples acquired at different times, e.g., while monitoring the progression, regression, remission, and/or recurrence of a disease or disorder in a subject. The sequence reads may be in any suitable file format, e.g., BCL, FASTA, FASTQ, etc. In some embodiments, sequencing data 122 is accessed by a sequencing data processing module 141, which performs various pre-processing, genome alignment, and demultiplexing operations, as described in detail below with reference to bioinformatics module 140. In some embodiments, sequence data that has been aligned to a reference construct, e.g., BAM file 124, is stored in test patient data store 120.

In some embodiments, the test patient data store 120 includes feature data 125, e.g., that is useful for identifying clinical support for personalized therapy. In some embodiments, the feature data 125 includes personal characteristics 126 of the patient, such as patient name, date of birth, gender, ethnicity, physical address, smoking status, alcohol consumption characteristic, anthropomorphic data, etc.

In some embodiments, the feature data 125 includes medical history data 127 for the patient, (e.g., date of initial disorder diagnosis, previous treatments and outcomes, adverse effects of therapy, therapy group history, clinical trial history, previous and current medications, surgical history, etc.), previous or current symptoms, previous or current therapies, previous treatment outcomes, previous disease diagnoses, diagnoses of depression, diagnoses of other physical or mental maladies, and family medical history. In some embodiments, the feature data 125 includes clinical features 128, such as pathology data 128-1, medical imaging data 128-2, and tissue culture and/or tissue organoid culture data 128-3.

In some embodiments, yet other clinical features, such as previous laboratory testing results, are stored in the test patient data store 120. Medical history data 127 and clinical features may be collected from various sources, including at intake directly from the patient, from an electronic medical record (EMR) or electronic health record (EHR) for the patient, or curated from other sources, such as fields from various testing records (e.g., genetic sequencing reports).

In some embodiments, the feature data 125 includes transcriptomic features 176, e.g., features extracted from RNA sequencing (RNA-seq) of mRNA, for the patient. As illustrated in FIG. 1C, non-limiting examples of transcriptomic features include boundary element data 177, including counts of the number of occurrences of each of a plurality of boundary elements in an RNA-Seq data set (e.g., counts of exon-exon, exon-intron, exon-promoter, promoter-promoter, intron-intron, exon-flanking nucleic acid, intron-flanking nucleic acid, and/or intron-noncoding sequence boundaries, or any junction not seen in the reference DNA genome) for each of a plurality of genes (e.g., genes 1 to 0 as illustrated in FIG. 1C), and gene expression data 178, including abundance levels for mRNA sequences in an RNA-seq dataset for each of a plurality of genes (e.g., genes 1 to 0 as illustrated in FIG. 1C).

In some embodiments, the feature data 125 includes genomic features 131 for the patient. Non-limiting examples of genomic features include allelic states 132 (e.g., the identity of alleles at one or more loci, support for wild type or variant alleles at one or more loci, support for SNVs/MNVs at one or more loci, support for indels at one or more loci, and/or support for gene rearrangements at one or more loci), methylation states 134 (e.g., a distribution of methylation patterns at one or more loci and/or support for aberrant methylation patterns at one or more loci), genomic copy numbers 135 (e.g., a copy number value at one or more loci and/or support for an aberrant (increased or decreased) copy number at one or more loci). In some embodiments, e.g., when the methods and systems described herein are used for precision oncology, the feature data includes one or more tumor-specific genomic features, e.g., allelic fractions (e.g., ratios of variant to reference alleles (or vice versa), tumor mutational burden (e.g., a measure of the number of mutations in the cancer genome of the subject), microsatellite instability status (e.g., a measure of the repeated unit length at one or more microsatellite loci and/or a classification of the MSI status for the patient's cancer), tumor ploidy, and homologous recombination deficiency (HRD) status.

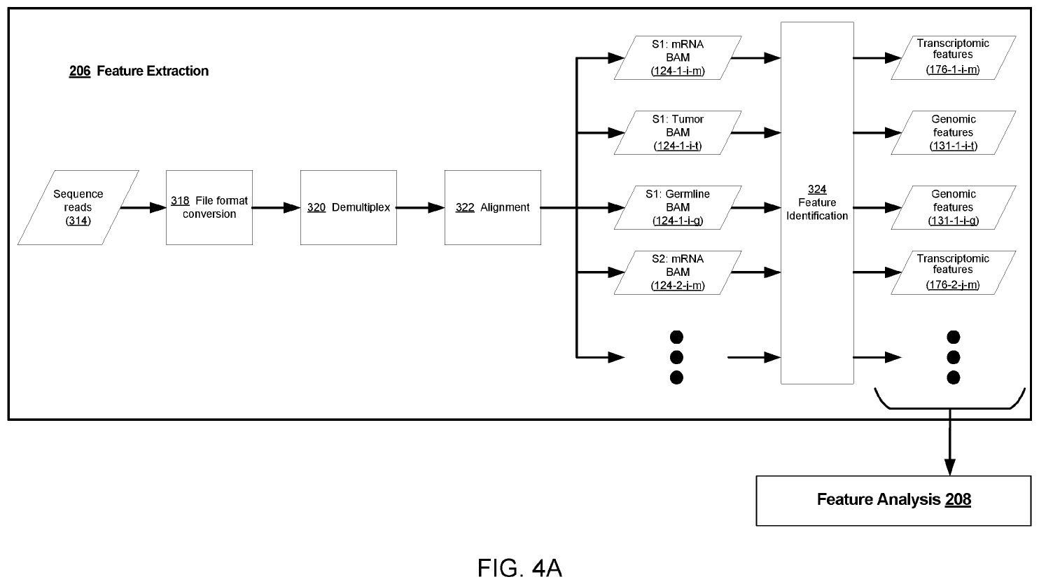

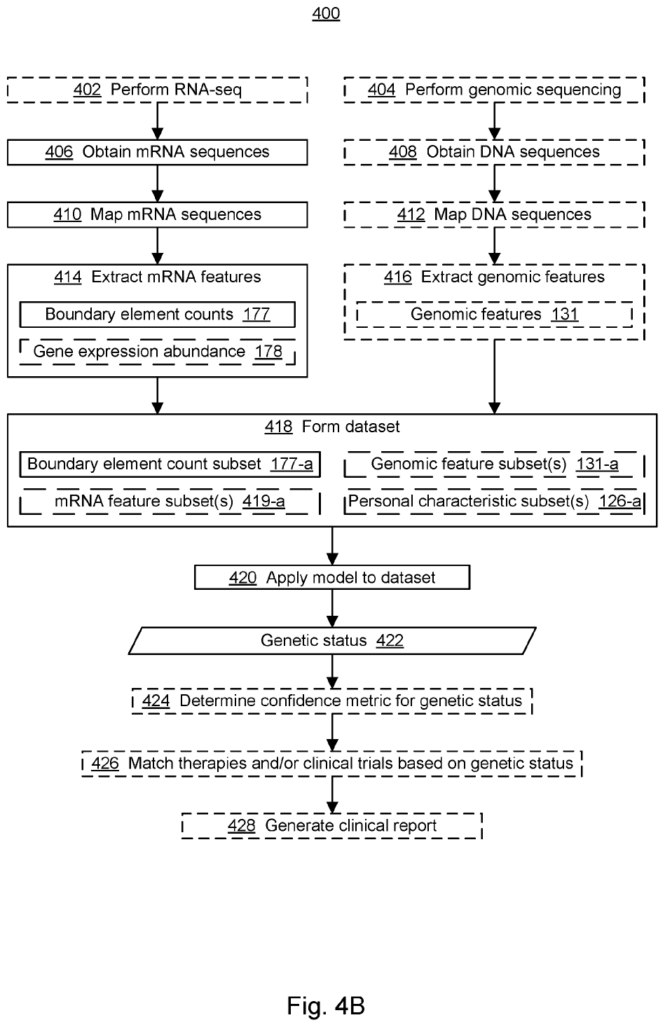

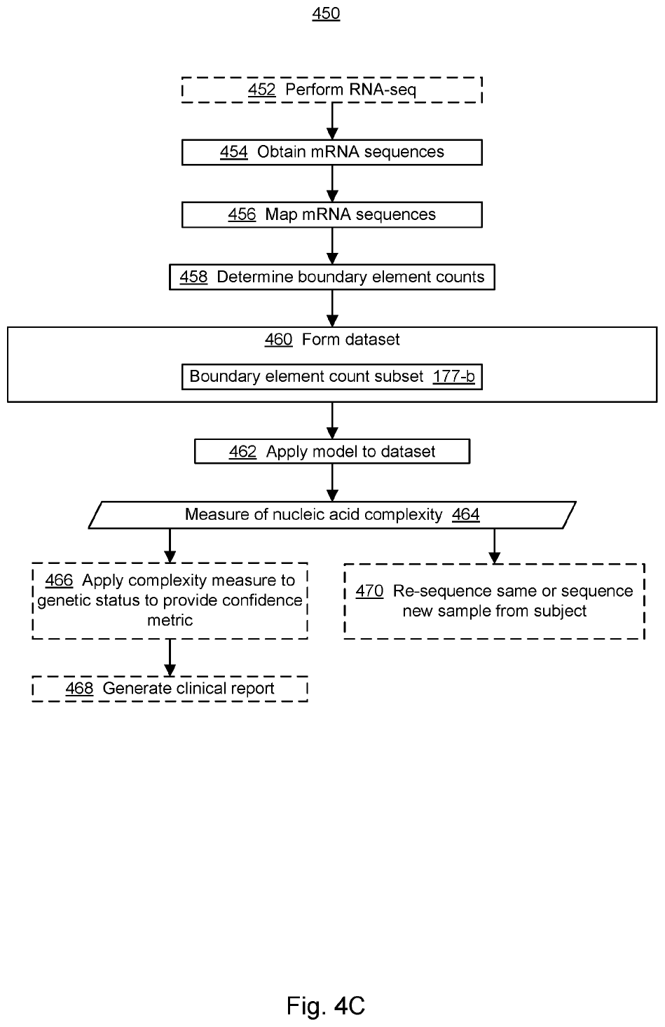

In some embodiments, one or more of the transcriptomic features 176 and/or genomic features 131 (e.g., that are used to determine a genetic status of a subject and/or evaluate the complexity of an RNA sequencing reaction through mRNA boundary analysis) are determined by a nucleic acid bioinformatics pipeline, e.g., as described in detail below with reference to FIG. 4. For example, in some embodiments, the feature data 125 include boundary element counts 177 (e.g., boundary element data 177-1 for Patient 1 121-1), as determined using a bioinformatics pipeline as described in further detail below with reference to FIGS. 1, 4, 5, and 7. In some embodiments, one or more of the genomic features 131 are obtained from an external source, e.g., not connected to the bioinformatics pipeline as described below.

For example, in some embodiments, data sets evaluated using the models described herein include all or a subset of the boundary element counts 177 determined for an RNA sequencing reaction. In some embodiments, the methods and systems for performing such methods determine more boundary element counts than are used in a particular model for determining a genetic status of a subject and/or evaluating the complexity of an RNA sequencing reaction. As such, in some embodiments, feature analysis module 160 selects a subset of the boundary element counts 177 stored in a test patient data store for analysis using a boundary element interpretation algorithm 171.

Referring again to FIG. 1B, in some embodiments, the feature data 125 further includes data 138 from other -omics fields of study. Non-limiting examples of -omics fields of study that may yield feature data useful for providing clinical support for personalized cancer therapy include transcriptomics, epigenomics, proteomics, metabolomics, metabonomics, microbiomics, lipidomics, glycomics, cellomics, and organoidomics.

In some embodiments, yet other features may include features derived from machine learning approaches, e.g., based at least in part on evaluation of any relevant molecular or clinical features, considered alone or in combination, not limited to those listed above. For instance, in some embodiments, one or more latent features learned from evaluation of cancer patient training datasets improve the diagnostic and prognostic power of the various analysis algorithms in the feature analysis module 160.

The skilled artisan will know of other types of features useful for providing clinical support for personalized therapy. The listing of features above is merely representative and should not be construed to be limiting.

In some embodiments, a test patient data store 120 includes clinical assessment data 139 for patients, e.g., based on the feature data 125 collected for the subject. In some embodiments, the clinical assessment data 139 includes a catalogue of actionable variants, actionable gene fusions, actionable mRNA isoform patterns, and/or other actionable characteristics 139-1 (e.g., genomic alterations such as CNV, focal CNV, SNV, MNV, as well as compound metrics thereof, known or believed to be targetable by one or more specific therapies), matched therapies 139-2 (e.g., the therapies known or believed to be particularly beneficial for treatment of subjects having actionable variants), and/or clinical reports 139-3 generated for the subject, e.g., based on identified actionable variants and characteristics 139-1, and/or matched therapies 139-2, and/or matched clinical trials.

In some embodiments, clinical assessment data 139 is generated by analysis of feature data 125 using the various algorithms of feature analysis module 160, as described in further detail below. In some embodiments, clinical assessment data 139 is generated, modified, and/or validated by evaluation of feature data 125 by a clinician, e.g., an oncologist. For instance, in some embodiments, a clinician (e.g., at clinical environment 220) uses feature analysis module 160, or accesses test patient data store 120 directly, to evaluate feature data 125 to make recommendations for personalized treatment of a patient. Similarly, in some embodiments, a clinician (e.g., at clinical environment 220) reviews recommendations determined using feature analysis module 160 and approves, rejects, or modifies the recommendations, e.g., prior to the recommendations being sent to a medical professional treating the patient.

Bioinformatics Module (140)

Referring again to FIG. 1A, the system (e.g., system 100) includes a bioinformatics module 140 that includes a feature extraction module 145 and optional ancillary data processing constructs, such as a sequence data processing module 141 and/or one or more reference sequence constructs 158 (e.g., a reference genome, exome, or targeted-panel construct that includes reference sequences for a plurality of loci targeted by a sequencing panel).

In some embodiments, bioinformatics module 140 includes a sequence data processing module 141 that includes instructions for processing sequence reads, e.g., raw sequence reads 123 from one or more sequencing reactions 122-i, prior to analysis by the various feature extraction algorithms, as described in detail below. In some embodiments, sequence data processing module 141 includes one or more pre-processing algorithms 142 that prepare the data for analysis. In some embodiments, the pre-processing algorithms 142 include instructions for converting the file format of the sequence reads from the output of the sequencer (e.g., a BCL file format) into a file format compatible with downstream analysis of the sequences (e.g., a FASTQ or FASTA file format). In some embodiments, the pre-processing algorithms 142 include instructions for evaluating the quality of the sequence reads (e.g., by interrogating quality metrics like Phred score, base-calling error probabilities, Quality (Q) scores, and the like) and/or removing sequence reads that do not satisfy a threshold quality (e.g., an inferred base call accuracy of at least 80%, at least 90%, at least 95%, at least 99%, at least 99.5%, at least 99.9%, or higher). In some embodiments, the pre-processing algorithms 142 include instructions for filtering the sequence reads for one or more properties, e.g., removing sequences failing to satisfy a lower or upper size threshold or removing duplicate sequence reads.

In some embodiments, sequence data processing module 141 includes one or more alignment algorithms 143, for aligning pre-processed sequence reads 123 to a reference sequence construct 158, e.g., a reference genome, exome, or targeted-panel construct. Many algorithms for aligning sequencing data to a reference construct are known in the art, for example, BWA, Blat, SHRiMP, LastZ, and MAQ. One example of a sequence read alignment package is the Burrows-Wheeler Alignment tool (BWA), which uses a Burrows-Wheeler Transform (BWT) to align short sequence reads against a large reference construct, allowing for mismatches and gaps. Li and Durbin, Bioinformatics, 25(14):1754-60 (2009), the content of which is incorporated herein by reference, in its entirety, for all purposes. Sequence read alignment packages import raw or pre-processed sequence reads 122, e.g., in BCL, FASTA, or FASTQ file formats, and output aligned sequence reads 124, e.g., in SAM or BAM file formats. Generally, any known alignment methodology, including pseudoalignment methodologies, find use in the methods and systems described herein.

In some embodiments, sequence data processing module 141 includes one or more demultiplexing algorithms 144, for dividing sequence read or sequence alignment files generated from sequencing reactions of pooled nucleic acids into separate sequence read or sequence alignment files, each of which corresponds to a different source of nucleic acids in the nucleic acid sequencing pool. For instance, because of the cost of sequencing reactions, it is common practice to pool nucleic acids from a plurality of samples into a single sequencing reaction. The nucleic acids from each sample are tagged with a sample-specific and/or molecule-specific sequence tag (e.g., a UMI), which is sequenced along with the molecule. In some embodiments, demultiplexing algorithms 144 sort these sequence tags in the sequence read or sequence alignment files to demultiplex the sequencing data into separate files for each of the samples included in the sequencing reaction.

Bioinformatics module 140 includes a feature extraction module 145, which includes instructions for identifying diagnostic features, e.g., transcriptomic features 176 and/or genomic features 131, from sequencing data 122 of biological samples from a subject. For instance, in some embodiments, boundary element identification module 153 identifies a boundary element (e.g., an exon-exon boundary element, gene fusion boundary element, focal deletion boundary element, etc.) through direct evidence (e.g., by identifying a sequence spanning the boundary element in a sequence read) or indirect evidence (e.g., by identifying sequences known to flank both sides of a boundary element in a sequence read; for instance, detection of a first sequence known to be from a first gene and a second sequence known to be from a second gene in a single RNA sequence read indicates that a gene fusion boundary element is present in the sequence read) by comparing a sequence from the sequencing data 122 to the known sequence of a boundary element (e.g., using direct evidence) or to sequences known to flank a boundary element (e.g., using indirect evidence) to determine counts for each of a plurality of different boundary elements.

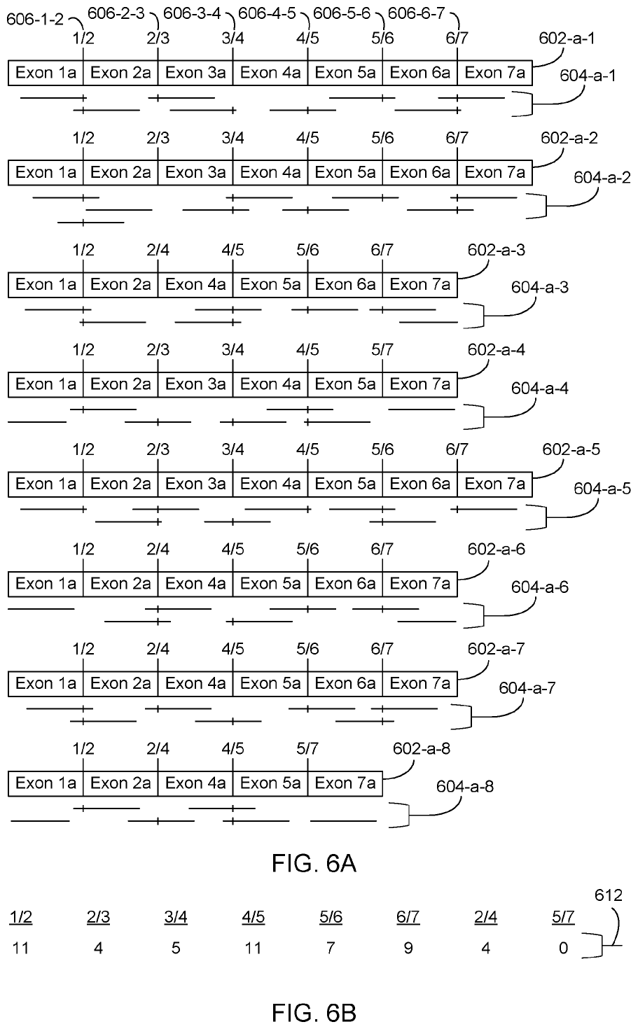

For example, FIG. 6A illustrates the portion of nucleic acid sequences 604 that map to a particular gene 602-a from an RNA sequencing reaction of a plurality of mRNA molecules 602-a-i of different isoforms. For ease of explanation, nucleic acid sequences 604 are illustrated in FIG. 6 as aligned to the specific mRNA molecule 602-a-i from which they were derived. However, because RNA-seq generates relatively short sequence reads, aligned sequencing data 124 only identifies the gene to which the sequence read 604 maps, not the particular mRNA molecule 602-a-i. Boundary element identification module 153 searches for a plurality of exon-exon boundary elements 606 (e.g., exon-exon boundary element 606-1-2 at the junction between exon 1 of gene 602-a and exon 2 of gene 602-a), present in the sequence reads 604 as denoted by the tick marks illustrated in FIG. 6. Boundary element identification module 153 then totals the number of each boundary element identified in sequencing data 122 (e.g., by advancing a dedicated counter upon each instance of the identification of the boundary element in the sequencing data 122), thereby determining boundary element counts 177 for each boundary element queried, e.g., boundary element counts 612 for gene 602-a.

In another example, feature extraction module 145 compares the identity of one or more nucleotides at a locus from the sequencing data 122 to the identity of the nucleotides at that locus in a reference sequence construct (e.g., a reference genome, exome, or targeted-panel construct) to determine whether the subject has a variant at that locus. In some embodiments, a feature extraction algorithm evaluates data other than the raw sequence, e.g., copy number, to identify a genomic alteration in the subject, e.g., a copy number variation (CNV).

For instance, in some embodiments, feature extraction module 145 includes one or more variant identification modules 146 that include instructions for various variant calling processes. In some embodiments, the variant identification module includes instructions for identifying one or more of nucleotide variants (e.g., single nucleotide variants (SNV) and multi-nucleotide variants (MNV)) using one or more SNV/MNV calling algorithms (e.g., algorithm 147), indels (e.g., insertions or deletions of nucleotides) using one or more indel calling algorithms (e.g., algorithm 148), and genomic rearrangements (e.g., inversions, translocation, and fusions of nucleotide sequences) using one or more genomic rearrangement calling algorithms (e.g., algorithm 149).

In some embodiments where the disease or disorder is a cancer, variants are identified in both the germline of the subject (e.g., germline variants) and in a cancer genome (e.g., somatic variants) of the subject, e.g., using the variant identification module 146. In some embodiments, separate germline and somatic variant identification modules are used, while in some embodiments they are integrated into a single module.

A SNV/MNV algorithm 147 may identify a substitution of a single nucleotide that occurs at a specific position in the genome. For example, at a specific base position, or locus, in the human genome, the C nucleotide may appear in most individuals, but in a minority of individuals, the position is occupied by an A. This means that there is a SNP at this specific position and the two possible nucleotide variations, C or A, are said to be alleles for this position. SNPs underlie differences in human susceptibility to a wide range of diseases (e.g., sickle-cell anemia, β-thalassemia and cystic fibrosis result from SNPs). The severity of illness and the way the body responds to treatments are also manifestations of genetic variations. For example, a single-base mutation in the APOE (apolipoprotein E) gene is associated with a lower risk for Alzheimer's disease. A single-nucleotide variant (SNV) is a variation in a single nucleotide without any limitations of frequency and may arise in somatic cells. A somatic single-nucleotide variation (e.g., caused by cancer) may also be called a single-nucleotide alteration. An MNP (Multiple-nucleotide polymorphisms) module may identify the substitution of consecutive nucleotides at a specific position in the genome.

An indel calling algorithm 148 may identify an insertion or deletion of bases in the genome of an organism classified among small genetic variations. While indels usually measure from 1 to 10 000 base pairs in length, a microindel is defined as an indel that results in a net change of 1 to 50 nucleotides. Indels can be contrasted with a SNP or point mutation. An indel inserts and/or deletes nucleotides from a sequence, while a point mutation is a form of substitution that replaces one of the nucleotides without changing the overall number in the DNA. Indels, being insertions and/or deletions, can be used as genetic markers in natural populations, especially in phylogenetic studies. Indel frequency tends to be markedly lower than that of single nucleotide polymorphisms (SNP), except near highly repetitive regions, including homopolymers and microsatellites.

A genomic rearrangement algorithm 149 may identify hybrid genes formed from two previously separate genes. It can occur as a result of translocation, interstitial deletion, or chromosomal inversion. Gene fusion can play an important role in tumorigenesis. Fusion genes can contribute to tumor formation because fusion genes can produce much more active abnormal protein than non-fusion genes. Often, fusion genes are oncogenes that cause cancer; these include BCR-ABL, TEL-AML1 (ALL with t(12; 21)), AML1-ETO (M2 AML with t(8; 21)), and TMPRSS2-ERG with an interstitial deletion on chromosome 21, often occurring in prostate cancer. In the case of TMPRSS2-ERG, by disrupting androgen receptor (AR) signaling and inhibiting AR expression by oncogenic ETS transcription factor, the fusion product regulates prostate cancer. Most fusion genes are found from hematological cancers, sarcomas, and prostate cancer. BCAM-AKT2 is a fusion gene that is specific and unique to high-grade serous ovarian cancer. Oncogenic fusion genes may lead to a gene product with a new or different function from the two fusion partners. Alternatively, a proto-oncogene is fused to a strong promoter, and thereby the oncogenic function is set to function by an upregulation caused by the strong promoter of the upstream fusion partner. The latter is common in lymphomas, where oncogenes are juxtaposed to the promoters of the immunoglobulin genes. Oncogenic fusion transcripts may also be caused by trans-splicing or read-through events. Since chromosomal translocations play such a significant role in neoplasia, a specialized database of chromosomal aberrations and gene fusions in cancer has been created. This database is called Mitelman Database of Chromosome Aberrations and Gene Fusions in Cancer.

In some embodiments where the disease or disorder is a cancer, feature extraction module 145 includes cancer-specific modules 150 (e.g., as illustrated in FIG. 1D) for identifying one or more complex genomic alterations (e.g., features that incorporate more than a change in the primary sequence of the genome) in a genome of the subject. For instance, in some embodiments, feature extraction module 145 includes modules for identifying one or more of variant allele fraction (e.g., variant allele fraction module 151), methylation status (e.g., methylation analysis module 152), microsatellite instability status (e.g., microsatellite instability analysis module 154), tumor mutational burden (e.g., tumor mutational burden analysis module 155), tumor ploidy (e.g., tumor ploidy analysis module 156), and homologous recombination pathway deficiencies (e.g., homologous recombination pathway analysis module 157).

Further details and specific embodiments regarding methods for determining a genetic status of a subject and/or evaluating the nucleic acid complexity of a nucleic acid sequencing reaction through mRNA boundary analysis are provided below with reference to FIGS. 4B, 4C, 5A-5G, 6, and 7A-7D.

Feature Analysis Module (160)

Referring again to FIG. 1A, the system (e.g., system 100) includes a feature analysis module 160 that includes one or more boundary element interpretation algorithms 171, e.g., an mRNA isoform analysis algorithm 172, genomic rearrangement analysis algorithm 173, disease state analysis algorithm 174, and/or sequence complexity analysis algorithm 175, one or more optional genomic alteration interpretation algorithms 161, one or more optional clinical data analysis algorithms 165, an optional therapeutic curation algorithm 165, and an optional recommendation validation module 167. In some embodiments, feature analysis module 160 identifies actionable variants and characteristics (e.g., gene fusions, mRNA isoforms and patterns thereof, genomic rearrangements, etc.) 139-1 and corresponding matched therapies 139-2 and/or clinical trials using one or more analysis algorithms (e.g., algorithms 162, 163, 164, and 165) to evaluate feature data 125. The identified actionable variants and characteristics 139-1 and corresponding matched therapies 139-2, which are optionally stored in test patient data store 120, are then curated by feature analysis module 160 to generate a clinical report 139-3, which is optionally validated by a user, e.g., a clinician, before being transmitted to a medical professional, e.g., an oncologist, treating the patient.

In some embodiments, the genomic alteration interpretation algorithms 161 include instructions for evaluating the effect that one or more transcriptomic features 176 and/or genomic features 131 of the subject, e.g., as identified by feature extraction module 145, have on the characteristics of the patient's medical condition (e.g., a disease or disorder such as cancer) and/or whether one or more personalized therapies may improve the clinical outcome for the patient. For example, in some embodiments, one or more genomic variant analysis algorithms 163 evaluate various transcriptomic features 176 and/or genomic alterations by querying a database, e.g., a look-up-table (“LUT”) of actionable mRNA isoforms, targeted therapies associated with the actionable transcriptomic feature and/or genomic alterations, and any other conditions that should be met before administering the targeted therapy to a subject having the actionable transcriptomic feature or genomic alteration. For instance, evidence suggests that depatuxizumab mafodotin (an anti-EGFR mAb conjugated to monomethyl auristatin F) has improved efficacy for the treatment of recurrent glioblastomas having EGFR focal amplifications. van den Bent M. et al., Cancer Chemother Pharmacol., 80(6):1209-17 (2017). Accordingly, the actionable genomic alteration LUT would have an entry for the focal amplification of the EGFR gene indicating that depatuxizumab mafodotin is a targeted therapy for glioblastomas (e.g., recurrent glioblastomas) having a focal gene amplification. In some instances, the LUT may also include counter indications for the associated targeted therapy, e.g., adverse drug interactions or personal characteristics that are counter-indicated for administration of the particular targeted therapy.

In some embodiments, a boundary element interpretation algorithm 171 determines whether a particular transcriptomic feature 176 should be reported to a medical professional treating the patient. In some embodiments, transcriptomic features 176 and/or genomic features 131 (e.g., genomic alterations and compound features) are reported when there is clinical evidence that the feature significantly impacts the biology of the disease or disorder, impacts the prognosis for the disease or disorder, and/or impacts pharmacogenomics, e.g., by indicating or counter-indicating particular therapeutic approaches. For instance, a boundary element interpretation algorithm 171 may classify the presence of a particular gene fusion or mRNA isoform as “Reportable,” e.g., meaning that the gene fusion or mRNA isoform has been identified as influencing the character of the disease or disorder, the overall disease state, and/or pharmacogenomics, as “Not Reportable,” e.g., meaning that the gene fusion or mRNA isoform has not been identified as influencing the character of the disease or disorder, the overall disease state, and/or pharmacogenomics, as “No Evidence,” e.g., meaning that no evidence exists supporting that the gene fusion or mRNA isoform is “Reportable” or “Not Reportable,” or as “Conflicting Evidence,” e.g., meaning that evidence exists supporting both that the gene fusion or mRNA isoform is “Reportable” and that the gene fusion or mRNA isoform is “Not Reportable.”

In some embodiments, the boundary element interpretation algorithms 171 include one or more feature analysis algorithms that evaluate a plurality of features to classify a disease or disorder, e.g., with respect to the effects of one or more targeted therapies. For instance, in some embodiments, feature analysis module 160 includes one or more models trained against transcriptomic feature data 176, one or more clinical therapies, and their associated clinical outcomes for a plurality of training subjects to classify a disease or disorder based on their predicted clinical outcomes following one or more therapies.

In some embodiments, the model is implemented as an artificial intelligence engine and may include gradient boosting models, random forest models, neural networks (NN), regression models, Naive Bayes models, and/or machine learning algorithms (MLA). An MLA or a NN may be trained from a training data set that includes one or more features 125, including transcriptomic feature data 176, personal characteristics 126, medical history 127, clinical features 128, genomic features 131, and/or other -omic features 138. MLAs include supervised algorithms (such as algorithms where the features/classifications in the data set are annotated) using linear regression, logistic regression, decision trees, classification and regression trees, naïve Bayes, nearest neighbor clustering; unsupervised algorithms (such as algorithms where no features/classification in the data set are annotated) using Apriori, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as mincut, harmonic function, manifold regularization), heuristic approaches, or support vector machines.

NNs include conditional random fields, convolutional neural networks, attention based neural networks, deep learning, long short term memory networks, or other neural models where the training data set includes a plurality of tumor samples, RNA expression data for each sample, and pathology reports covering imaging data for each sample.

While MLA and neural networks identify distinct approaches to machine learning, the terms may be used interchangeably herein. Thus, a mention of MLA may include a corresponding NN or a mention of NN may include a corresponding MLA unless explicitly stated otherwise. Training may include providing optimized datasets, labeling these traits as they occur in patient records, and training the MLA to predict or classify based on new inputs. Artificial NNs are efficient computing models which have shown their strengths in solving hard problems in artificial intelligence. They have also been shown to be universal approximators, that is, they can represent a wide variety of functions when given appropriate parameters.

In some embodiments, the optional genomic alteration interpretation algorithms 161 include one or more pathogenic variant analysis algorithms 162, which evaluate various genomic features to identify the presence of a pathogen associated with the patient's disease or disorder and/or targeted therapies associated with a pathogenic infection in the disease or disorder. For instance, RNA expression patterns of some cancers are associated with the presence of an oncogenic pathogen that is helping to drive the cancer. See, for example, U.S. patent application Ser. No. 16/802,126, filed Feb. 26, 2020, the content of which is hereby incorporated by reference, in its entirety, for all purposes. In some instances, the recommended therapy for the disease or disorder is different when the disease or disorder is associated with the pathogenic infection than when it is not. In some embodiments, one or more pathogenic variant analysis algorithms 162 evaluate RNA abundance data 178 to determine whether a signature exists in the data that indicates the presence of the pathogen in the disease or disorder. Similarly, in some embodiments, bioinformatics module 140 includes an algorithm that searches for the presence of pathogenic nucleic acid sequences in sequencing data 122. See, for example, U.S. Provisional Patent Application Ser. No. 62/978,067, filed Feb. 18, 2020, the content of which is hereby incorporated by reference, in its entirety, for all purposes. Accordingly, in some embodiments, one or more pathogenic variant analysis algorithms 162 evaluates whether the presence of a pathogen in a subject is associated with an actionable therapy. In some embodiments, system 100 queries a database, e.g., a look-up-table (“LUT”), of actionable pathogenic infections, targeted therapies associated with the actionable infections, and any other conditions that should be met before administering the targeted therapy to a subject that is infected with the pathogen. In some instances, the LUT may also include counter indications for the associated targeted therapy, e.g., adverse drug interactions or personal characteristics that are counter-indicated for administration of the particular targeted therapy.

In some embodiments, system 100 includes a model training module that includes instructions for training one or more untrained or partially trained models based on feature data from a training dataset. In some embodiments, system 100 also includes a database of training data for use in training the one or more classifiers. In other embodiments, the classifier training module accesses a remote storage device hosting training data. In some embodiments, the training data includes a set of training features, including but not limited to, various types of the feature data 125 illustrated in FIG. 1B. In some embodiments, the classifier training module uses patient data 121, e.g., when test patient data store 120 also stores a record of treatments administered to the patient and patient outcomes following therapy.

In some embodiments, feature analysis module 160 includes one or more clinical data analysis algorithms 165, which evaluate clinical features 128 of a disease or disorder to identify targeted therapies which may benefit the subject. For example, in some embodiments, e.g., where feature data 125 includes pathology data 128-1, one or more clinical data analysis algorithms 165 evaluate the data to determine whether an actionable therapy is indicated based on the histopathology of a tumor biopsy from the subject, e.g., which is indicative of a particular cancer type and/or stage of cancer. In some embodiments, system 100 queries a database, e.g., a look-up-table (“LUT”), of actionable clinical features (e.g., pathology features), targeted therapies associated with the actionable features, and any other conditions that should be met before administering the targeted therapy to a subject associated with the actionable clinical features 128 (e.g., pathology features 128-1). In some embodiments, system 100 evaluates the clinical features 128 (e.g., pathology features 128-1) directly to determine whether the patient's disease or disorder is sensitive to a particular therapeutic agent. Further details on example methods, systems, and algorithms for classifying cancer and identifying targeted therapies based on clinical data, such as pathology data 128-1, imaging data 138-2, and/or tissue culture/organoid data 128-3 are discussed, for example, in U.S. patent application Ser. No. 16/830,186, filed on Mar. 25, 2020, U.S. patent application Ser. No. 16/789,363, filed on Feb. 12, 2020, and U.S. patent application Ser. No. 17/227,120, filed on Apr. 9, 2021, the contents of which are hereby incorporated by reference, in their entireties, for all purposes.

In some embodiments, feature analysis module 160 includes a clinical trials module that evaluates test patient data 121 to determine whether the patient is eligible for inclusion in a clinical trial for treatment of a disease or disorder, e.g., a clinical trial that is currently recruiting patients, a clinical trial that has not yet begun recruiting patients, and/or an ongoing clinical trial that may recruit additional patients in the future. In some embodiments, a clinical trial module evaluates test patient data 121 to determine whether the results of a clinical trial are relevant for the patient, e.g., the results of an ongoing clinical trial and/or the results of a completed clinical trial. For instance, in some embodiments, system 100 queries a database, e.g., a look-up-table (“LUT”) of clinical trials, e.g., active and/or completed clinical trials, and compares patient data 121 with inclusion criteria for the clinical trials, stored in the database, to identify clinical trials with inclusion criteria that closely match and/or exactly match the patient's data 121. In some embodiments, a record of matching clinical trials, e.g., those clinical trials that the patient may be eligible for and/or that may inform personalized treatment decisions for the patient, are stored in clinical assessment database 139.

In some embodiments, feature analysis module 160 includes a therapeutic curation algorithm 166 that assembles actionable variants and characteristics 139-1, matched therapies 139-2, and/or relevant clinical trials identified for the patient, as described above. In some embodiments, a therapeutic curation algorithm 166 evaluates certain criteria related to which actionable variants and characteristics 139-1, matched therapies 139-2, and/or relevant clinical trials should be reported and/or whether certain matched therapies, considered alone or in combination, may be counter-indicated for the patient, e.g., based on personal characteristics 126 of the patient and/or known drug-drug interactions. In some embodiments, the therapeutic curation algorithm then generates one or more clinical reports 139-3 for the patient. In some embodiments, the therapeutic curation algorithm generates a first clinical report 139-3-1 that is to be reported to a medical professional treating the patient and a second clinical report 139-3-2 that will not be communicated to the medical professional but may be used to improve various algorithms within the system.

In some embodiments, feature analysis module 160 includes a recommendation validation module 167 that includes an interface allowing a clinician to review, modify, and approve a clinical report 139-3 prior to the report being sent to a medical professional, e.g., an oncologist, treating the patient.

In some embodiments, each of the one or more feature collections, sequencing modules, bioinformatics modules (including, e.g., alteration module(s), structural variant calling and data processing modules), classification modules and outcome modules are communicatively coupled to a data bus to transfer data between each module for processing and/or storage. In some alternative embodiments, each of the feature collection, alteration module(s), structural variant and feature store are communicatively coupled to each other for independent communication without sharing the data bus.

Further details on systems and exemplary embodiments of modules and feature collections are discussed in PCT Application PCT/US19/69149, titled “A METHOD AND PROCESS FOR PREDICTING AND ANALYZING PATIENT COHORT RESPONSE, PROGRESSION, AND SURVIVAL,” filed Dec. 31, 2019, the content of which is incorporated herein by reference, in its entirety, for all purposes.

### EXAMPLE EMBODIMENTS

Now that details of a system 100 for providing clinical support for personalized therapy have been disclosed, e.g., with improved determination of a genetic status of a subject and/or with improved determination of the nucleic acid complexity of a nucleic acid sequencing reaction, through various forms of mRNA boundary analysis, are provided below. Specifically, example processes are described below with reference to FIGS. 2A-2B, 3, 4A-4C, 5A-5G, and 7A-7D. In some embodiments, such processes and features of the system are carried out by modules 118, 120, 140, 160, and/or 180, as illustrated in FIG. 1A. Referring to these methods, the systems described herein (e.g., system 100) include instructions for determining a genetic status of a subject and/or evaluating the nucleic acid complexity of a nucleic acid sequencing reaction that are improved compared to conventional methods.

FIG. 2B: Distributed Diagnostic and Clinical Environment

In some aspects, the methods described herein for providing clinical support for a disease or disorder are performed across a distributed diagnostic/clinical environment, e.g., as illustrated in FIG. 2B. However, in some embodiments, the improved methods described herein for supporting clinical decisions in personalized (e.g., by determining a genetic status of a subject and/or evaluating the nucleic acid complexity of a nucleic acid sequencing reaction) are performed at a single location, e.g., at a single computing system or environment, although ancillary procedures supporting the methods described herein, and/or procedures that make further use of the results of the methods described herein, may be performed across a distributed diagnostic/clinical environment.

FIG. 2B illustrates an example of a distributed diagnostic/clinical environment 210. In some embodiments, the distributed diagnostic/clinical environment is connected via communication network 105. In some embodiments, one or more biological samples are collected from a subject in clinical environment 220, e.g., a doctor's office, hospital, or medical clinic, or at a home health care environment (not depicted). In some embodiments, one or more biological samples, or portions thereof, are processed within the clinical environment 220 where collection occurred, using a processing device 224, e.g., a nucleic acid sequencer for obtaining sequencing data, a microscope for obtaining pathology data, a mass spectrometer for obtaining proteomic data, etc. In some embodiments, one or more biological samples, or portions thereof, are sent to one or more external environments, e.g., sequencing lab 230, pathology lab 240, and/or molecular biology lab 250, each of which includes a processing device 234, 244, and 254, respectively, to generate biological data 121 for the subject. Each environment includes a communications device 222, 232, 242, and 252, respectively, for communicating biological data 121 about the subject to a processing server 262 and/or database 264, which may be located in yet another environment, e.g., processing/storage center 260. Thus, in some embodiments, different portions of the systems and methods described herein are fulfilled by different processing devices located in different physical environments.

Accordingly, in some embodiments, a method for providing clinical support for personalized therapy, e.g., with improved determination of a genetic status of a subject and/or with improved determination of the nucleic acid complexity of a nucleic acid sequencing reaction, through various forms of mRNA boundary analysis, is performed across one or more environments, as illustrated in FIG. 2B. For instance, in some such embodiments, a sample is collected at clinical environment 220 or in a home healthcare environment. The sample, or a portion thereof, is sent to sequencing lab 230 where raw sequence reads 123 of nucleic acids in the sample are generated by sequencer 234. The raw sequencing data 123 is communicated, e.g., from communications device 232, to database 264 at processing/storage center 260, where processing server 262 extracts features from the sequence reads by executing one or more of the processes in bioinformatics module 140, thereby generating genomic features 131 for the sample. Processing server 262 may then analyze the identified features by executing one or more of the processes in feature analysis module 160, thereby generating clinical assessment 139, including a clinical report 139-3. A clinician may access clinical report 139-3, e.g., at processing/storage center 260 or through communications network 105, via recommendation validation module 167. After final approval, clinical report 139-3 is transmitted to a medical professional, e.g., an oncologist, at clinical environment 220, who uses the report to support clinical decision making for personalized treatment of the patient.

FIG. 2A: Example Workflow

FIG. 2A is a flowchart of an example workflow 200 for collecting and analyzing data in order to generate a clinical report 139 to support clinical decision making in personalized medicine. Advantageously, the methods described herein improve this process, for example, by improving various stages within feature extraction 206, including determining a genetic status of a subject and/or analyzing the nucleic acid complexity of a nucleic acid sequencing reaction, through various forms of mRNA boundary analysis. Workflow 200 is tailored for a precision oncology application, but the skilled artisan will know how to tailor such workflows to provide clinical support for other diseases and disorders.

Briefly, the workflow begins with patient intake and sample collection 201, where one or more liquid biopsy samples, one or more tumor biopsy, and one or more normal and/or control tissue samples are collected from the patient (e.g., at a clinical environment 220 or home healthcare environment, as illustrated in FIG. 2B). In some embodiments, personal data 126 corresponding to the patient and a record of the one or more biological samples obtained (e.g., patient identifiers, patient clinical data, sample type, sample identifiers, cancer conditions, etc.) are entered into a data analysis platform, e.g., test patient data store 120. Accordingly, in some embodiments, the methods disclosed herein include obtaining one or more biological samples from one or more subjects, e.g., cancer patients. In some embodiments, the subject is a human, e.g., a human cancer patient.

In some embodiments, one or more of the biological samples obtained from the patient is a biological liquid sample, also referred to as a liquid biopsy sample. In some embodiments, one or more of the biological samples obtained from the patient are selected from blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. In some embodiments, the liquid biopsy sample includes blood and/or saliva. In some embodiments, the liquid biopsy sample is peripheral blood. In some embodiments, blood samples are collected from patients in commercial blood collection containers, e.g., using a PAXgene® Blood DNA Tubes. In some embodiments, saliva samples are collected from patients in commercial saliva collection containers, e.g., using an Oragene® DNA Saliva Kit.

In some embodiments, one or more biological samples collected from the patient is a solid tissue sample, e.g., a solid tumor sample or a solid normal tissue sample. Methods for obtaining solid tissue samples, e.g., of cancerous and/or normal tissue are known in the art and are dependent upon the type of tissue being sampled. For example, bone marrow biopsies and isolation of circulating tumor cells can be used to obtain samples of blood cancers, endoscopic biopsies can be used to obtain samples of cancers of the digestive tract, bladder, and lungs, needle biopsies (e.g., fine-needle aspiration, core needle aspiration, vacuum-assisted biopsy, and image-guided biopsy, can be used to obtain samples of subdermal tumors, skin biopsies, e.g., shave biopsy, punch biopsy, incisional biopsy, and excisional biopsy, can be used to obtain samples of dermal cancers, and surgical biopsies can be used to obtain samples of cancers affecting internal organs of a patient. In some embodiments, a solid tissue sample is a formalin-fixed tissue (FFT). In some embodiments, a solid tissue sample is a macro-dissected formalin fixed paraffin embedded (FFPE) tissue. In some embodiments, a solid tissue sample is a fresh frozen tissue sample.

In some embodiments, a dedicated normal sample is collected from the patient, for co-processing with a liquid biopsy sample. Generally, the normal sample is of a non-cancerous tissue, and can be collected using any tissue collection means described above. In some embodiments, buccal cells collected from the inside of a patient's cheeks are used as a normal sample. Buccal cells can be collected by placing an absorbent material, e.g., a swab, in the subject's mouth and rubbing it against their cheek, e.g., for at least 15 second or for at least 30 seconds. The swab is then removed from the patient's mouth and inserted into a tube, such that the tip of the tube is submerged into a liquid that serves to extract the buccal cells off of the absorbent material. An example of buccal cell recovery and collection devices is provided in U.S. Pat. No. 9,138,205, the content of which is hereby incorporated by reference, in its entirety, for all purposes. In some embodiments, the buccal swab DNA is used as a source of normal DNA in circulating heme malignancies.

The biological samples collected from the patient are, optionally, sent to various analytical environments (e.g., sequencing lab 230, pathology lab 240, and/or molecular biology lab 250) for processing (e.g., data collection) and/or analysis (e.g., feature extraction). Wet lab processing 204 may include cataloguing samples (e.g., accessioning), examining clinical features of one or more samples (e.g., pathology review), and nucleic acid sequence analysis (e.g., extraction, library prep, capture+hybridize, pooling, and sequencing). In some embodiments, the workflow includes clinical analysis of one or more biological samples collected from the subject, e.g., at a pathology lab 240 and/or a molecular and cellular biology lab 250, to generate clinical features such as pathology features 128-3, imaging data 128-3, and/or tissue culture/organoid data 128-3.

In some embodiments, the pathology data 128-1 collected during clinical evaluation includes visual features identified by a pathologist's inspection of a specimen (e.g., a solid tumor biopsy), e.g., of stained H&E or IHC slides. In some embodiments, the sample is a solid tissue biopsy sample. In some embodiments, the tissue biopsy sample is a formalin-fixed tissue (FFT), e.g., a formalin-fixed paraffin-embedded (FFPE) tissue. In some embodiments, the tissue biopsy sample is an FFPE or FFT block. In some embodiments, the tissue biopsy sample is a fresh-frozen tissue biopsy. The tissue biopsy sample can be prepared in thin sections (e.g., by cutting and/or affixing to a slide), to facilitate pathology review (e.g., by staining with immunohistochemistry stain for IHC review and/or with hematoxylin and eosin stain for H&E pathology review). For instance, analysis of slides for H&E staining or IHC staining may reveal features such as tumor infiltration, programmed death-ligand 1 (PD-L1) status, human leukocyte antigen (HLA) status, or other immunological features.

In some embodiments, a liquid sample (e.g., blood) collected from the patient (e.g., in EDTA-containing collection tubes) is prepared on a slide (e.g., by smearing) for pathology review. In some embodiments, macrodissected FFPE tissue sections, which may be mounted on a histopathology slide, from solid tissue samples (e.g., tumor or normal tissue) are analyzed by pathologists. In some embodiments, tumor samples are evaluated to determine, e.g., the tumor purity of the sample, the percent tumor cellularity as a ratio of tumor to normal nuclei, etc. For each section, background tissue may be excluded or removed such that the section meets a tumor purity threshold, e.g., where at least 20% of the nuclei in the section are tumor nuclei, or where at least 25%, 30%, 40%, 50%, 60%, 70%, 80%, 90%, or more of the nuclei in the section are tumor nuclei.

Further details on methods, systems, and algorithms for using pathology data to classify cancer and identify targeted therapies are discussed, for example, in are discussed, for example, in U.S. patent application Ser. No. 16/830,186, filed on Mar. 25, 2020, and U.S. patent application Ser. No. 17/227,120, filed on Apr. 9, 2021, the contents of which are hereby incorporated by reference, in their entireties, for all purposes.

In some embodiments, imaging data 128-2 collected during clinical evaluation includes features identified by review of in vitro and/or in vivo imaging results (e.g., of a tumor site), for example a size of a tumor, tumor size differentials over time (such as during treatment or during other periods of change). In some embodiments, imaging data 128-2 includes features determined using machine learning algorithms to evaluate imaging data collected as described above.

Further details on methods, systems, and algorithms for using medical imaging to classify cancer and identify targeted therapies are discussed, for example, in are discussed, for example, in U.S. patent application Ser. No. 16/830,186, filed on Mar. 25, 2020, and U.S. patent application Ser. No. 17/227,120, filed on Apr. 9, 2021, the contents of which are hereby incorporated by reference, in their entireties, for all purposes.

In some embodiments, tissue culture/organoid data 128-3 collected during clinical evaluation includes features identified by evaluation of cultured tissue from the subject. For instance, in some embodiments, tissue samples obtained from the patients (e.g., tumor tissue, normal tissue, or both) are cultured (e.g., in liquid culture, solid-phase culture, and/or organoid culture) and various features, such as cell morphology, growth characteristics, genomic alterations, and/or drug sensitivity, are evaluated. In some embodiments, tissue culture/organoid data 128-3 includes features determined using machine learning algorithms to evaluate tissue culture/organoid data collected as described above. Examples of tissue organoid (e.g., personal tumor organoid) culturing and feature extractions thereof are described in PCT/US20/56930, filed on Oct. 22, 2020, and U.S. patent application Ser. No. 16/693,117, filed on Nov. 22, 2019, the contents of which are hereby incorporated by reference, in their entireties, for all purposes.

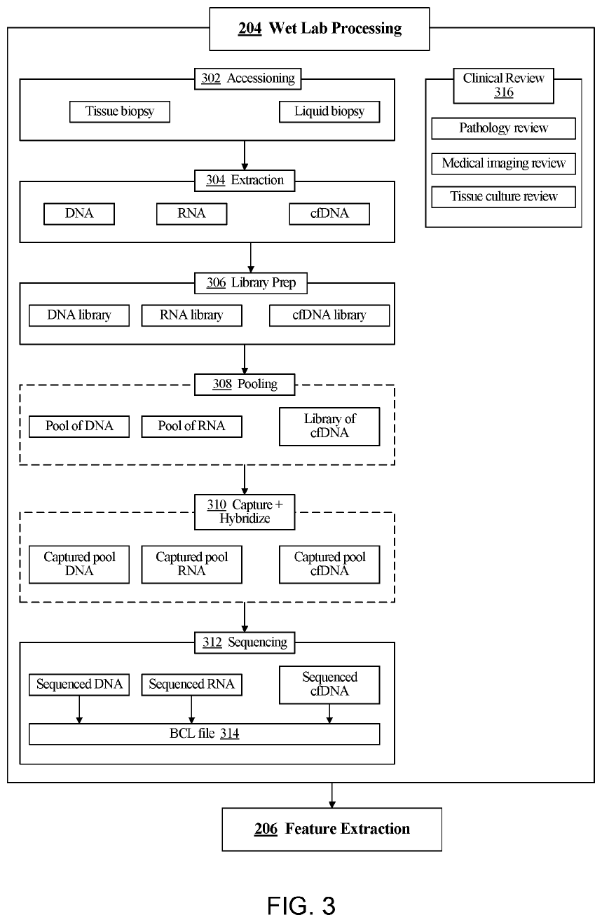

Nucleic acid sequencing of one or more samples collected from the subject is performed, e.g., at sequencing lab 230, during wet lab processing 204. An example workflow for nucleic acid sequencing is illustrated in FIG. 3. In some embodiments, the one or more biological samples obtained at the sequencing lab 230 are accessioned (302), to track the sample and data through the sequencing process.

Next, nucleic acids, e.g., RNA and/or DNA are extracted (304) from the one or more biological samples. Methods for isolating nucleic acids from biological samples are known in the art and are dependent upon the type of nucleic acid being isolated (e.g., cfDNA, DNA, and/or RNA) and the type of sample from which the nucleic acids are being isolated (e.g., liquid biopsy samples, white blood cell buffy coat preparations, formalin-fixed paraffin-embedded (FFPE) solid tissue samples, and fresh frozen solid tissue samples). The selection of any particular nucleic acid isolation technique for use in conjunction with the embodiments described herein is well within the skill of the person having ordinary skill in the art, who will consider the sample type, the state of the sample, the type of nucleic acid being sequenced, and the sequencing technology being used.

For instance, many techniques for DNA isolation, e.g., genomic DNA isolation, from a tissue sample are known in the art, such as organic extraction, silica adsorption, and anion exchange chromatography. Likewise, many techniques for RNA isolation, e.g., mRNA isolation, from a tissue sample are known in the art. For example, acid guanidinium thiocyanate-phenol-chloroform extraction (see, for example, Chomczynski and Sacchi, 2006, Nat Protoc, 1(2):581-85, which is hereby incorporated by reference herein), and silica bead/glass fiber adsorption (see, for example, Poeckh, T. et al., 2008, Anal Biochem., 373(2):253-62, which is hereby incorporated by reference herein). The selection of any particular DNA or RNA isolation technique for use in conjunction with the embodiments described herein is well within the skill of the person having ordinary skill in the art, who will consider the tissue type, the state of the tissue, e.g., fresh, frozen, formalin-fixed, paraffin-embedded (FFPE), and the type of nucleic acid analysis that is to be performed.

In some embodiments, isolated DNA molecules are mechanically sheared to an average length using an ultrasonicator (for example, a Covaris ultrasonicator). In some embodiments, isolated nucleic acid molecules are analyzed to determine their fragment size, e.g., through gel electrophoresis techniques and/or the use of a device such as a LabChip GX Touch. The skilled artisan will know of an appropriate range of fragment sizes, based on the sequencing technique being employed, as different sequencing techniques have differing fragment size requirements for robust sequencing. In some embodiments, quality control testing is performed on the extracted nucleic acids (e.g., DNA and/or RNA), e.g., to assess the nucleic acid concentration and/or fragment size. For example, sizing of DNA fragments provides valuable information used for downstream processing, such as determining whether DNA fragments require additional shearing prior to sequencing.

Wet lab processing 204 then includes preparing a nucleic acid library from the isolated nucleic acids (e.g., cfDNA, DNA, and/or RNA). For example, in some embodiments, DNA libraries (e.g., gDNA and/or cfDNA libraries) are prepared from isolated DNA from the one or more biological samples. In some embodiments, the DNA libraries are prepared using a commercial library preparation kit, e.g., the KAPA Hyper Prep Kit, a New England Biolabs (NEB) kit, or a similar kit.

In some embodiments, during library preparation, adapters (e.g., UDI adapters, such as Roche SeqCap dual end adapters, or UMI adapters such as full length or stubby Y adapters) are ligated onto the nucleic acid molecules. In some embodiments, the adapters include unique molecular identifiers (UMIs), which are short nucleic acid sequences (e.g., 3-10 base pairs) that are added to ends of DNA fragments during adapter ligation. In some embodiments, UMIs are degenerate base pairs that serve as a unique tag that can be used to identify sequence reads originating from a specific DNA fragment. In some embodiments, e.g., when multiplex sequencing will be used to sequence DNA from a plurality of samples (e.g., from the same or different subjects) in a single sequencing reaction, a patient-specific index is also added to the nucleic acid molecules. In some embodiments, the patient specific index is a short nucleic acid sequence (e.g., 3-20 nucleotides) that are added to ends of DNA fragments during library construction, that serve as a unique tag that can be used to identify sequence reads originating from a specific patient sample. Examples of identifier sequences are described, for example, in Kivioj a et al., Nat. Methods 9(1):72-74 (2011) and Islam et al., Nat. Methods 11(2):163-66 (2014), the contents of which are hereby incorporated by reference, in their entireties, for all purposes.

In some embodiments, an adapter includes a PCR primer landing site, designed for efficient binding of a PCR or second-strand synthesis primer used during the sequencing reaction. In some embodiments, an adapter includes an anchor binding site, to facilitate binding of the DNA molecule to anchor oligonucleotide molecules on a sequencer flow cell, serving as a seed for the sequencing process by providing a starting point for the sequencing reaction. During PCR amplification following adapter ligation, the UMIs, patient indexes, and binding sites are replicated along with the attached DNA fragment. This provides a way to identify sequence reads that came from the same original fragment in downstream analysis.

In some embodiments, DNA libraries are amplified and purified using commercial reagents, (e.g., Axygen MAG PCR clean up beads). In some such embodiments, the concentration and/or quantity of the DNA molecules are then quantified using a fluorescent dye and a fluorescence microplate reader, standard spectrofluorometer, or filter fluorometer. In some embodiments, library amplification is performed on a device (e.g., an Illumina C-Bot2) and the resulting flow cell containing amplified target-captured DNA libraries is sequenced on a next generation sequencer (e.g., an Illumina HiSeq 4000 or an Illumina NovaSeq 6000) to a unique on-target depth selected by the user. In some embodiments, DNA library preparation is performed with an automated system, using a liquid handling robot (e.g., a SciClone NGSx).

In some embodiments, where feature data 125 includes methylation states 132 for one or more genomic locations, nucleic acids isolated from the biological sample (e.g., cfDNA and/or DNA) are treated to convert unmethylated cytosines to uracils, e.g., prior to generating the sequencing library. Accordingly, when the nucleic acids are sequenced, all cytosines called in the sequencing reaction were necessarily methylated, since the unmethylated cytosines were converted to uracils and accordingly would have been called as thymidines, rather than cytosines, in the sequencing reaction. Commercial kits are available for bisulfite-mediated conversion of methylated cytosines to uracils, for instance, the EZ DNA Methylation™-Gold, EZ DNA Methylation™-Direct, and EZ DNA Methylation™-Lightning kit (available from Zymo Research Corp (Irvine, CA)). Commercial kits are also available for enzymatic conversion of methylated cytosines to uracils, for example, the APOBEC-Seq kit (available from NEBiolabs, Ipswich, MA).

In some embodiments, wet lab processing 204 includes pooling (308) DNA molecules from a plurality of libraries, corresponding to different samples from the same and/or different patients, to forming a sequencing pool of DNA libraries. When the pool of DNA libraries is sequenced, the resulting sequence reads correspond to nucleic acids isolated from multiple samples. The sequence reads can be separated into different sequence read files, corresponding to the various samples represented in the sequencing read based on the unique identifiers present in the added nucleic acid fragments. In this fashion, a single sequencing reaction can generate sequence reads from multiple samples. Advantageously, this allows for the processing of more samples per sequencing reaction.

In some embodiments, wet lab processing 204 includes enriching (310) a sequencing library, or pool of sequencing libraries, for target nucleic acids, e.g., nucleic acids encompassing loci that are informative for precision oncology and/or used as internal controls for the sequencing or bioinformatics processes. In some embodiments, enrichment is achieved by hybridizing target nucleic acids in the sequencing library to probes that hybridize to the target sequences, and then isolating the captured nucleic acids away from off-target nucleic acids that are not bound by the capture probes. In some embodiments, one or more off-target nucleic acids will remain in the final sequencing pool.

Advantageously, enriching for target sequences prior to sequencing nucleic acids significantly reduces the costs and time associated with sequencing, facilitates multiplex sequencing by allowing multiple samples to be mixed together for a single sequencing reaction, and significantly reduces the computation burden of aligning the resulting sequence reads, as a result of significantly reducing the total amount of nucleic acids analyzed from each sample.

In some embodiments, the enrichment is performed prior to pooling multiple nucleic acid sequencing libraries. However, in other embodiments, the enrichment is performed after pooling nucleic acid sequencing libraries, which has the advantage of reducing the number of enrichment assays that have to be performed.

In some embodiments, the enrichment is performed prior to generating a nucleic acid sequencing library. This has the advantage that fewer reagents are needed to perform both the enrichment (because there are fewer target sequences at this point, prior to library amplification) and the library production (because there are fewer nucleic acid molecules to tag and amplify after the enrichment). However, this raises the possibility of pull-down bias and/or that small variations in the enrichment protocol will result in less consistent results.

In some embodiments, nucleic acid libraries are pooled (two or more DNA libraries may be mixed to create a pool) and treated with reagents to reduce off-target capture, for example Human COT-1 and/or IDT xGen Universal Blockers. Pools may be dried in a vacufuge and resuspended. DNA libraries or pools may be hybridized to a probe set (for example, a probe set specific to a panel that includes loci from at least 100, 600, 1,000, 10,000, etc. of the 19,000 known human genes) and amplified with commercially available reagents (for example, the KAPA HiFi HotStart ReadyMix). For example, in some embodiments, a pool is incubated in an incubator, PCR machine, water bath, or other temperature-modulating device to allow probes to hybridize. Pools may then be mixed with Streptavidin-coated beads or another means for capturing hybridized DNA-probe molecules, such as DNA molecules representing exons of the human genome and/or genes selected for a genetic panel.

Pools may be amplified and purified more than once using commercially available reagents, for example, the KAPA HiFi Library Amplification kit and Axygen MAG PCR clean up beads, respectively. The pools or DNA libraries may be analyzed to determine the concentration or quantity of DNA molecules, for example by using a fluorescent dye (for example, PicoGreen pool quantification) and a fluorescence microplate reader, standard spectrofluorometer, or filter fluorometer. In one example, the DNA library preparation and/or capture is performed with an automated system, using a liquid handling robot (for example, a SciClone NGSx).

In some embodiments, e.g., where a whole genome sequencing method will be used, nucleic acid sequencing libraries are not target-enriched prior to sequencing, in order to obtain sequencing data on substantially all of the competent nucleic acids in the sequencing library. Similarly, in some embodiments, e.g., where a whole genome sequencing method will be used, nucleic acid sequencing libraries are not mixed, because of bandwidth limitations related to obtaining significant sequencing depth across an entire genome. However, in other embodiments, e.g., where a low-pass whole genome sequencing (LPWGS) methodology will be used, nucleic acid sequencing libraries can still be pooled, because very low average sequencing coverage is achieved across a respective genome, e.g., between about 0.5× and about 5×.

In some embodiments, a plurality of nucleic acid probes (e.g., a probe set) is used to enrich one or more target sequences in a nucleic acid sample (e.g., an isolated nucleic acid sample or a nucleic acid sequencing library), e.g., where one or more target sequences is informative for precision oncology. For instance, in some embodiments, one or more of the target sequences encompasses a locus that is associated with an actionable allele. That is, variations of the target sequence are associated with targeted therapeutic approaches. In some embodiments, one or more of the target sequences and/or a property of one or more of the target sequences is used in a classifier trained to distinguish two or more cancer states.

In some embodiments, the probe set includes probes targeting one or more gene loci, e.g., exon or intron loci. In some embodiments, the probe set includes probes targeting one or more loci not encoding a protein, e.g., regulatory loci, miRNA loci, and other non-coding loci, e.g., that have been found to be associated with cancer. In some embodiments, the plurality of loci includes at least 25, 50, 100, 150, 200, 250, 300, 350, 400, 500, 750, 1000, 2500, 5000, or more human genomic loci. In some embodiments, the probe set is a whole exome sequencing panel.

Generally, probes for enrichment of nucleic acids include DNA, RNA, or a modified nucleic acid structure with a base sequence that is complementary to a locus of interest. For instance, a probe designed to hybridize to a locus in a DNA molecule can contain a sequence that is complementary to either strand, because the DNA molecules are double stranded. In some embodiments, each probe in the plurality of probes includes a nucleic acid sequence that is identical or complementary to at least 10, at least 11, at least 12, at least 13, at least 14, or at least 15 consecutive bases of a locus of interest. In some embodiments, each probe in the plurality of probes includes a nucleic acid sequence that is identical or complementary to at least 20, 25, 30, 40, 50, 75, 100, 150, 200, or more consecutive bases of a locus of interest.

Targeted panels provide several benefits for nucleic acid sequencing. For example, in some embodiments, algorithms for discriminating between, e.g., a first and second disease or disorder condition can be trained on smaller, more informative data sets (e.g., fewer genes), which leads to more computationally efficient training of classifiers that discriminate between the first and second cancer states. Such improvements in computational efficiency, owing to the reduced size of the discriminating gene set, can advantageously either be used to speed up classifier training or be used to improve the performance of such classifiers (e.g., through more extensive training of the classifier).

In some embodiments, the gene panel is a whole-exome panel that analyzes the exomes of a biological sample. In some embodiments, the gene panel is a whole genome panel that analyzes the genome of a specimen.

In some embodiments, the probes include additional nucleic acid sequences that do not share any homology to the locus of interest. For example, in some embodiments, the probes also include nucleic acid sequences containing an identifier sequence, e.g., a unique molecular identifier (UMI), e.g., that is unique to a particular sample or subject. Examples of identifier sequences are described, for example, in Kivioj a et al., 2011, Nat. Methods 9(1), pp. 72-74 and Islam et al., 2014, Nat. Methods 11(2), pp. 163-66, which are incorporated by reference herein. Similarly, in some embodiments, the probes also include primer nucleic acid sequences useful for amplifying the nucleic acid molecule of interest, e.g., using PCR. In some embodiments, the probes also include a capture sequence designed to hybridize to an anti-capture sequence for recovering the nucleic acid molecule of interest from the sample.

Likewise, in some embodiments, the probes each include a non-nucleic acid affinity moiety covalently attached to nucleic acid molecule that is complementary to the locus of interest, for recovering the nucleic acid molecule of interest. Non-limited examples of non-nucleic acid affinity moieties include biotin, digoxigenin, and dinitrophenol. In some embodiments, the probe is attached to a solid-state surface or particle, e.g., a dipstick or magnetic bead, for recovering the nucleic acid of interest. In some embodiments, the methods described herein include amplifying the nucleic acids that bound to the probe set prior to further analysis, e.g., sequencing. Methods for amplifying nucleic acids, e.g., by PCR, are well known in the art.

Sequence reads are then generated (312) from the sequencing library or pool of sequencing libraries. Sequencing data may be acquired by any methodology known in the art. For example, next generation sequencing (NGS) techniques such as sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators. In some embodiments, sequencing is performed using next generation sequencing technologies, such as short-read technologies. In other embodiments, long-read sequencing or another sequencing method known in the art is used.

Next-generation sequencing produces millions of short reads (e.g., sequence reads) for each biological sample. Accordingly, in some embodiments, the plurality of sequence reads obtained by next-generation sequencing of nucleic acid molecules are DNA sequence reads. In some embodiments, the sequence reads have an average length of at least fifty nucleotides. In other embodiments, the sequence reads have an average length of at least 50, 60, 70, 80, 90, 100, 150, 200, 250, 300, or more nucleotides.

In some embodiments, sequencing is performed after enriching for nucleic acids (e.g., cfDNA, gDNA, and/or RNA) encompassing a plurality of predetermined target sequences, e.g., human genes and/or non-coding sequences associated with cancer. Advantageously, sequencing a nucleic acid sample that has been enriched for target nucleic acids, rather than all nucleic acids isolated from a biological sample, significantly reduces the average time and cost of the sequencing reaction. Accordingly, in some embodiments, the methods described herein include obtaining a plurality of sequence reads of nucleic acids that have been hybridized to a probe set for hybrid-capture enrichment (e.g., of one or more genes listed in Table 1, List 1, and/or List 2).

In some embodiments, panel-targeting sequencing is performed to an average on-target depth of at least 30×, at least 40×, at least 50×, at least 60×, at least 70×, at least 80×, at least 90×, at least 100×, at least 500×, at least 750×, at least 1000×, at least 2500×, at least 500×, at least 10,000×, or greater depth. In some embodiments, samples are further assessed for uniformity above a sequencing depth threshold (e.g., 95% of all targeted base pairs at 300× sequencing depth). In some embodiments, the sequencing depth threshold is a minimum depth selected by a user or practitioner.

In some embodiments, the sequence reads are obtained by a whole genome sequencing methodology. As described herein, the whole genome sequencing is performed at lower sequencing depth than smaller target-panel sequencing reactions, because many more loci are being sequenced. For example, in some embodiments, whole genome sequencing is performed to an average sequencing depth of at least 0.2×, at least 0.5×, at least 1×, at least 1.5×, at least 2×, at least 2.5×, at least 3×, at least 3.5×, at least 4×, at least 4.5×, or greater. In some embodiments, whole genome sequencing is performed to an average sequencing depth of no more than 7.5×, no more than 7×, no more than 6.5×, no more than 6×, no more than 5.5×, no more than 5×, no more than 4.5×, no more than 4×, no more than 3.5×, no more than 3×, no more than 2.5×, no more than 2×, no more than 1.5×, no more than 1×, or less. In some embodiments, low-pass whole genome sequencing (LPWGS) is performed to an average sequencing depth of about 0.25× to about 5×, or to an average sequencing depth of about 0.5× to about 5×, or to an average sequencing depth of about 1× to about 5×, or to an average sequencing depth of about 2× to about 5×, or to an average sequencing depth of about 3× to about 5×, or to an average sequencing depth of about 1× to about 4×, or to an average sequencing depth of about 1× to about 3×, or to an average sequencing depth of about 1.5× to about 4×, or to an average sequencing depth of about 1.5× to about 3×, or to an average sequencing depth of about 2× to about 3×.

In some embodiments, the raw sequence reads resulting from the sequencing reaction are output from the sequencer in a native file format, e.g., a BCL file. In some embodiments, the native file is passed directly to a bioinformatics pipeline (e.g., variant analysis 206), components of which are described in detail below. In other embodiments, pre-processing is performed prior to passing the sequences to the bioinformatics platform. For instance, in some embodiments, the format of the sequence read file is converted from the native file format (e.g., BCL) to a file format compatible with one or more algorithms used in the bioinformatics pipeline (e.g., FASTQ or FASTA). In some embodiments, the raw sequence reads are filtered to remove sequences that do not meet one or more quality thresholds. In some embodiments, raw sequence reads generated from the same unique nucleic acid molecule in the sequencing read are collapsed into a single sequence read representing the molecule, e.g., using UMIs as described above. In some embodiments, one or more of these pre-processing activities is performed within the bioinformatics pipeline itself.

In one example, a sequencer may generate a BCL file. A BCL file may include raw image data of a plurality of patient specimens which are sequenced. BCL image data is an image of the flow cell across each cycle during sequencing. A cycle may be implemented by illuminating a patient specimen with a specific wavelength of electromagnetic radiation, generating a plurality of images which may be processed into base calls via BCL to FASTQ processing algorithms which identify which base pairs are present at each cycle. The resulting FASTQ file includes the entirety of reads for each patient specimen paired with a quality metric, e.g., in a range from 0 to 64 where a 64 is the best quality and a 0 is the worst quality. In embodiments where both a diseased tissue sample and a non-diseased tissue sample are sequenced, sequence reads in the corresponding FASTQ files may be matched, such that a diseased-normal analysis may be performed.

FASTQ format is a text-based format for storing both a biological sequence, such as a nucleotide sequence, and its corresponding quality scores. These FASTQ files are analyzed to determine what genetic variants or copy number changes are present in the sample. Each FASTQ file contains reads that may be paired-end or single reads and may be short-reads or long-reads, where each read represents one detected sequence of nucleotides in a nucleic acid molecule that was isolated from the patient sample or a copy of the nucleic acid molecule, detected by the sequencer. Each read in the FASTQ file is also associated with a quality rating. The quality rating may reflect the likelihood that an error occurred during the sequencing procedure that affected the associated read. In some embodiments, the results of paired-end sequencing of each isolated nucleic acid sample are contained in a split pair of FASTQ files, for efficiency. Thus, in some embodiments, forward (Read 1) and reverse (Read 2) sequences of each isolated nucleic acid sample are stored separately but in the same order and under the same identifier.

In various embodiments, the bioinformatics pipeline may filter FASTQ data from the corresponding sequence data file for each respective biological sample. Such filtering may include correcting or masking sequencer errors and removing (trimming) low quality sequences or bases, adapter sequences, contaminations, chimeric reads, overrepresented sequences, biases caused by library preparation, amplification, or capture, and other errors.

While workflow 200 illustrates obtaining a biological sample, extracting nucleic acids from the biological sample, and sequencing the isolated nucleic acids, in some embodiments, sequencing data used in the improved systems and methods described herein (e.g., which include improved methods for determining copy number variation status) is obtained by receiving previously generated sequence reads, in electronic form.

FIG. 4A illustrates an example bioinformatics pipeline 206 (e.g., as used for feature extraction in the various workflows illustrated in the Figures and described herein) for providing clinical support for treatment of a disease or disorder. As shown in FIG. 4A, sequencing data 122 obtained from the wet lab processing 204 (e.g., sequence reads 314) is input into the pipeline. The pipeline may detect RNA boundary elements, expression abundance data, SNVs, INDELs, copy number amplifications/deletions and genomic rearrangements (for example, fusions). The pipeline may employ unique molecular index (UMI)-based consensus base calling as a method of error suppression as well as a Bayesian tri-nucleotide context-based position level error suppression. In various embodiments, it is able to detect variants having a 0.1%, 0.15%, 0.2%, 0.25%, 0.3%, 0.4%, or 0.5% variant allele fraction.

In some embodiments, the sequencing data is processed (e.g., using sequence data processing module 141) to prepare it for genomic feature identification 385. For instance, in some embodiments as described above, the sequencing data is present in a native file format provided by the sequencer. Accordingly, in some embodiments, the system (e.g., system 100) applies a pre-processing algorithm 142 to convert the file format (318) to one that is recognized by one or more upstream processing algorithms. For example, BCL file outputs from a sequencer can be converted to a FASTQ file format using the bcl2fastq or bcl2fastq2 conversion software (Illumina®). FASTQ format is a text-based format for storing both a biological sequence, such as nucleotide sequence, and its corresponding quality scores. These FASTQ files are analyzed to determine what genetic variants, copy number changes, etc., are present in the sample.

In some embodiments, other preprocessing functions are performed, e.g., filtering sequence reads 122 based on a desired quality, e.g., size and/or quality of the base calling. In some embodiments, quality control checks are performed to ensure the data is sufficient for variant calling. For instance, entire reads, individual nucleotides, or multiple nucleotides that are likely to have errors may be discarded based on the quality rating associated with the read in the FASTQ file, the known error rate of the sequencer, and/or a comparison between each nucleotide in the read and one or more nucleotides in other reads that has been aligned to the same location in the reference genome. Filtering may be done in part or in its entirety by various software tools, for example, a software tool such as Skewer. See, Jiang, H. et al., BMC Bioinformatics 15(182):1-12 (2014). FASTQ files may be analyzed for rapid assessment of quality control and reads, for example, by a sequencing data QC software such as AfterQC, Kraken, RNA-SeQC, FastQC, or another similar software program. For paired end reads, reads may be merged.

In some embodiments, two FASTQ output files are generated, e.g., one for RNA-seq and one for genomic sequencing. If two or more patient samples are processed simultaneously on the same sequencer flow cell, e.g., an RNA-seq reaction and a genomic sequencing reaction, a difference in the sequence of the adapters used for each patient sample barcodes nucleic acids extracted from both samples, to associate each read with the correct patient sample and facilitate assignment to the correct FASTQ file.

For efficiency, in some embodiments, the results of paired-end sequencing of each isolate are contained in a split pair of FASTQ files. Forward (Read 1) and reverse (Read 2) sequences of each sequencing run are stored separately but in the same order and under the same identifier. In various embodiments, the bioinformatics pipeline may filter FASTQ data from each isolate. Such filtering may include correcting or masking sequencer errors and removing (trimming) low quality sequences or bases, adapter sequences, contaminations, chimeric reads, overrepresented sequences, biases caused by library preparation, amplification, or capture, and other errors.

Similarly, in some embodiments, sequencing (312) is performed on a pool of nucleic acid sequencing libraries prepared from different biological samples, e.g., from the same or different patients. Accordingly, in some embodiments, the system demultiplexes (320) the data (e.g., using demultiplexing algorithm 144) to separate sequence reads into separate files for each sequencing library included in the sequencing pool, e.g., based on UMI or patient identifier sequences added to the nucleic acid fragments during sequencing library preparation, as described above. In some embodiments, the demultiplexing algorithm is part of the same software package as one or more pre-processing algorithms 142. For instance, the bcl2fastq or bcl2fastq2 conversion software (Illumina®) include instructions for both converting the native file format output from the sequencer and demultiplexing sequence reads 122 output from the reaction.

In some embodiments, the sequence reads are then aligned (322), e.g., using an alignment algorithm 143, to a reference sequence construct 158, e.g., a reference genome, reference exome, reference transcriptome, or other reference construct prepared for a particular targeted-panel sequencing reaction. For example, in some embodiments, individual sequence reads 123, in electronic form (e.g., in FASTQ files), are aligned against a reference sequence construct for the species of the subject (e.g., a reference human transcriptome) by identifying a sequence in a region of the reference sequence construct that best matches the sequence of nucleotides in the sequence read. In some embodiments, the sequence reads are aligned to a reference exome, reference transcriptome, or reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. Any of a variety of alignment tools can be used for this task.

For instance, local sequence alignment algorithms compare subsequences of different lengths in the query sequence (e.g., sequence read) to subsequences in the subject sequence (e.g., reference construct) to create the best alignment for each portion of the query sequence. In contrast, global sequence alignment algorithms align the entirety of the sequences, e.g., end to end. Examples of local sequence alignment algorithms include the Smith-Waterman algorithm (see, for example, Smith and Waterman, J Mol. Biol., 147(1):195-97 (1981), which is incorporated herein by reference), Lalign (see, for example, Huang and Miller, Adv. Appl. Math, 12:337-57 (1991), which is incorporated by reference herein), and PatternHunter (see, for example, Ma B. et al., Bioinformatics, 18(3):440-45 (2002), which is incorporated by reference herein).

In some embodiments, the read mapping process starts by building an index of either the reference genome or the reads, which is then used to retrieve the set of positions in the reference sequence where the reads are more likely to align. Once this subset of possible mapping locations has been identified, alignment is performed in these candidate regions with slower and more sensitive algorithms. See, for example, Hatem et al., 2013, “Benchmarking short sequence mapping tools,” BMC Bioinformatics 14: p. 184; and Flicek and Birney, 2009, “Sense from sequence reads: methods for alignment and assembly,” Nat Methods 6(Suppl. 11), S6-S12, each of which is hereby incorporated by reference. In some embodiments, the mapping tools methodology makes use of a hash table or a Burrows-Wheeler transform (BWT). See, for example, Li and Homer, 2010, “A survey of sequence alignment algorithms for next-generation sequencing,” Brief Bioinformatics 11, pp. 473-483, which is hereby incorporated by reference.

Other software programs designed to align reads include, for example, Novoalign (Novocraft, Inc.), Bowtie, Burrows Wheeler Aligner (BWA), and/or programs that use a Smith-Waterman algorithm. Candidate reference genomes include, for example, hg19, GRCh38, hg38, GRCh37, and/or other reference genomes developed by the Genome Reference Consortium. In some embodiments, the alignment generates a SAM file, which stores the locations of the start and end of each read according to coordinates in the reference genome and the coverage (number of reads) for each nucleotide in the reference genome.

For example, in some embodiments, each read of a FASTQ file is aligned to a location in the human genome having a sequence that best matches the sequence of nucleotides in the read. There are many software programs designed to align reads, for example, Novoalign (Novocraft, Inc.), Bowtie, Burrows Wheeler Aligner (BWA), programs that use a Smith-Waterman algorithm, etc. Alignment may be directed using a reference genome (for example, hg19, GRCh38, hg38, GRCh37, other reference genomes developed by the Genome Reference Consortium, etc.) by comparing the nucleotide sequences in each read with portions of the nucleotide sequence in the reference genome to determine the portion of the reference genome sequence that is most likely to correspond to the sequence in the read. In some embodiments, one or more SAM files are generated for the alignment, which store the locations of the start and end of each read according to coordinates in the reference genome and the coverage (number of reads) for each nucleotide in the reference genome. The SAM files may be converted to BAM files. In some embodiments, the BAM files are sorted, and duplicate reads are marked for deletion, resulting in de-duplicated BAM files.

In some embodiments, adapter-trimmed FASTQ files are aligned to the 19th edition of the human reference genome build (HG19) using Burrows-Wheeler Aligner (BWA, Li and Durbin, Bioinformatics, 25(14):1754-60 (2009). Following alignment, reads are grouped by alignment position and UMI family and collapsed into consensus sequences, for example, using fgbio tools (e.g., available on the internet at fulcrumgenomics.github.io/fgbio/). Bases with insufficient quality or significant disagreement among family members (for example, when it is uncertain whether the base is an adenine, cytosine, guanine, etc.) may be replaced by N's to represent a wildcard nucleotide type. PHRED scores are then scaled based on initial base calling estimates combined across all family members. Following single-strand consensus generation, duplex consensus sequences are generated by comparing the forward and reverse oriented PCR products with mirrored UMI sequences. In various embodiments, a consensus can be generated across read pairs. Otherwise, single-strand consensus calls will be used. Following consensus calling, filtering is performed to remove low-quality consensus fragments. The consensus fragments are then re-aligned to the human reference genome using BWA. A BAM output file is generated after the re-alignment, then sorted by alignment position, and indexed.

In some embodiments, this process produces a BAM file for the RNA-seq reaction (e.g., mRNA BAM 124-1-i-m), and optionally a tumor genomic BAM (e.g., Tumor BAM 124-1-i-t) and/or germline genomic BAM (e.g., Germline BAM 124-1-i-g), as illustrated in FIG. 4A. In various embodiments, BAM files may be analyzed to detect boundary elements, expression abundance levels, genetic variants and other genetic features, including single nucleotide variants (SNVs), copy number variants (CNVs), gene rearrangements, etc.

In some embodiments, the sequencing data is normalized, e.g., to account for pull-down, amplification, and/or sequencing bias (e.g., mappability, GC bias etc.). See, for example, Schwartz et al., PLoS ONE 6(1):e16685 (2011) and Benjamini and Speed, Nucleic Acids Research 40(10):e72 (2012), the contents of which are hereby incorporated by reference, in their entireties, for all purposes.

In some embodiments, SAM files generated after alignment are converted to BAM files 124. Thus, after preprocessing sequencing data generated for a pooled sequencing reaction, BAM files are generated for each of the sequencing libraries present in the master sequencing pools. In some embodiments, one or more samples acquired from one or more additional subjects at time j (e.g., mRNA BAM 124-2-j-m corresponding to alignments of sequence reads of nucleic acids isolated from a sample from subject 2). In some embodiments, BAM files are sorted, and duplicate reads are marked for deletion, resulting in de-duplicated BAM files. For example, tools like SamBAMBA mark and filter duplicate alignments in the sorted BAM files.

Generally, the methods and systems described herein are independent and, thus, not reliant upon any particular sequencing data generation methods, e.g., sample preparation, sequencing, and/or data pre-processing methodologies. However, in some embodiments, the methods described below include one or more features 204 of generating sequencing data, as illustrated in FIGS. 2A and 3.

Alignment files prepared as described above (e.g., BAM files 124) are then passed to a feature extraction module 145, where the sequences are analyzed (324) to identify transcriptomic features (e.g., boundary element counts, expression abundance levels, etc.), genomic alterations (e.g., SNVs/MNVs, indels, genomic rearrangements, copy number variations, etc.), and/or determine various characteristics of the patient's disease or disorder. Many software packages for identifying genomic alterations are known in the art, for example, freebayes, PolyBayse, samtools, GATK, pindel, SAMtools, Breakdancer, Cortex, Crest, Delly, Gridss, Hydra, Lumpy, Manta, and Socrates. For a review of many of these variant calling packages see, for example, Cameron, D. L. et al., Nat. Commun., 10(3240):1-11 (2019), the content of which is hereby incorporated by reference, in its entirety, for all purposes. Generally, these software packages identify variants in sorted SAM or BAM files 124, relative to one or more reference sequence constructs 158. The software packages then output a file e.g., a raw VCF (variant call format), listing the variants (e.g., genomic features 131) called and identifying their location relevant to the reference sequence construct (e.g., where the sequence of the sample nucleic acids differ from the corresponding sequence in the reference construct). In some embodiments, system 100 digests the contents of the native output file to populate feature data 125 in test patient data store 120. In other embodiments, the native output file serves as the record of these genomic features 131 in test patient data store 120.

Generally, the systems described herein can employ any combination of available variant calling software packages and internally developed variant identification algorithms. In some embodiments, the output of a particular algorithm of a variant calling software is further evaluated, e.g., to improve variant identification. Accordingly, in some embodiments, system 100 employs an available variant calling software package to perform some of all of the functionality of one or more of the algorithms shown in feature extraction module 145.

In various aspects, the detected genetic variants and genetic features are analyzed as a form of quality control. For example, a pattern of detected genetic variants or features may indicate an issue related to the sample, sequencing procedure, and/or bioinformatics pipeline (e.g., example, contamination of the sample, mislabeling of the sample, a change in reagents, a change in the sequencing procedure and/or bioinformatics pipeline, etc.).

Generally, any combination of the modules and algorithms of feature extraction module 145, e.g., illustrated in FIG. 1A, can be used for a bioinformatics pipeline used in conjunction with the methods and systems described herein. For instance, in some embodiments, an architecture useful for the methods and systems described herein includes at least one of the modules (e.g., boundary element identification module 153) shown in feature extraction module 145. In some embodiments, an architecture includes at least 2, 3, 4, 5, 6, 7, 8, 9, 10, or more of the modules or algorithms shown in feature extraction module 145. Further, in some embodiments, feature extraction modules and/or algorithms not illustrated in FIG. 1A find use in the methods and systems described herein.

Quality Control

In some embodiments, a positive sensitivity control sample is processed and sequenced along with one or more clinical samples. In some embodiments, the control sample is included in at least one flow cell of a multi-flow cell reaction and is processed and sequenced each time a set of samples is sequenced or periodically throughout the course of a plurality of sets of samples. In some embodiments, the control includes a pool of controls. In some embodiments, a quality control analysis requires that read metrics of variants present in the control sample fall within acceptable criteria. In some embodiments, a quality control requires approval by a pathologist before the results are reported. Examples of criteria used for such purpose are described, for example, in WO 2021/168146.

Feature Characterization

In some embodiments, a predicted functional effect and/or clinical interpretation for one or more identified features and/or genetic statuses is curated by using information from databases. In some embodiments, a weighted-heuristic model is used to characterize each feature and/or genetic status.

In some embodiments, identified features and/or genetic statuses are labeled as “potentially actionable,” “biologically relevant,” “variants of unknown significance (VUSs),” or “benign.” Potentially actionable features and/or genetic statuses have an associated therapy based on evidence from the medical literature. Biologically relevant features and/or genetic statuses may have functional significance or have been observed in the medical literature but are not associated with a specific therapy. Features and/or genetic statuses of unknown significance exhibit an unclear effect on function and/or without sufficient evidence to determine their pathogenicity. In some embodiments, benign variants are not reported.

For instance, in some embodiments, feature and/or genetic status evaluation and reporting is performed, where detected features and/or genetic statuses are investigated following criteria from known evolutionary models, functional data, clinical data, literature, and other research endeavors, e.g., including tumor organoid experiments. In some embodiments, features and/or genetic statuses are prioritized and classified based on known feature/status-disease relationships, hotspot regions within genes, internal and external somatic databases, primary literature, and other features of somatic drivers. Features and/or genetic statuses can be added to a patient (or sample, for example, organoid sample) report based on recommendations from the AMP/ASCO/CAP guidelines. Additional guidelines may be followed. Briefly, features and/or genetic statuses with therapeutic, diagnostic, or prognostic significance may be prioritized in the report. Non-actionable features and/or genetic statuses may be included as biologically relevant, followed by variants of uncertain significance. Translocations may be reported based on features of known gene fusions, relevant breakpoints, and biological relevance. Evidence may be curated from public and private databases or research and presented as 1) consensus guidelines 2) clinical research, or 3) case studies, with a link to the supporting literature. Germline alterations may be reported as secondary findings in a subset of genes for consenting patients. These may include genes recommended by the ACMG and additional genes associated with cancer predisposition or drug resistance.

In some embodiments, a clinical report 139-3 includes information about clinical trials for which the patient is eligible, therapies that are specific to the patient's disease or disorder, and/or possible therapeutic adverse effects associated with the specific characteristics of the patient's disease or disorder, e.g., the patient's genetic variations, epigenetic abnormalities, associated oncogenic pathogenic infections, and/or pathology abnormalities, or other characteristics of the patient's sample and/or clinical records. For example, in some embodiments, the clinical report includes such patient information and analysis metrics, including diagnosis, patient demographic and/or institution, matched therapies (e.g., FDA approved and/or investigational), matched clinical trials, variants of unknown significance (VUS), genes with low coverage, panel information, specimen information, details on reported variants, patient clinical history, status and/or availability of previous test results, and/or version of bioinformatics pipeline.

In some embodiments, the results included in the report, and/or any additional results (for example, from the bioinformatics pipeline), are used to query a database of clinical data, for example, to determine whether there is a trend showing that a particular therapy was effective or ineffective in treating (e.g., slowing or halting cancer progression), and/or adverse effects of such treatments in other patients having the same or similar characteristics.

As illustrated in FIG. 2A, in some embodiments, a clinical report is checked for final validation, review, and sign-off by a medical practitioner. The clinical report is then sent to a clinician treating the patient.

Genetic Status Determination

In some embodiments, the analysis of aligned sequence reads, e.g., in SAM or BAM format, includes determination of a genetic status for the subject (e.g., a gene fusion or other genomic rearrangement status, an mRNA isoform status or profile, and/or a disease characterization) using one or more boundary element interpretation algorithms 171. For example, FIG. 4B illustrates a workflow of an exemplary method 400 for determining a genetic status of a subject, e.g., to support clinical decision making in treating a disease or disorder, in accordance with some embodiments of the present disclosure.

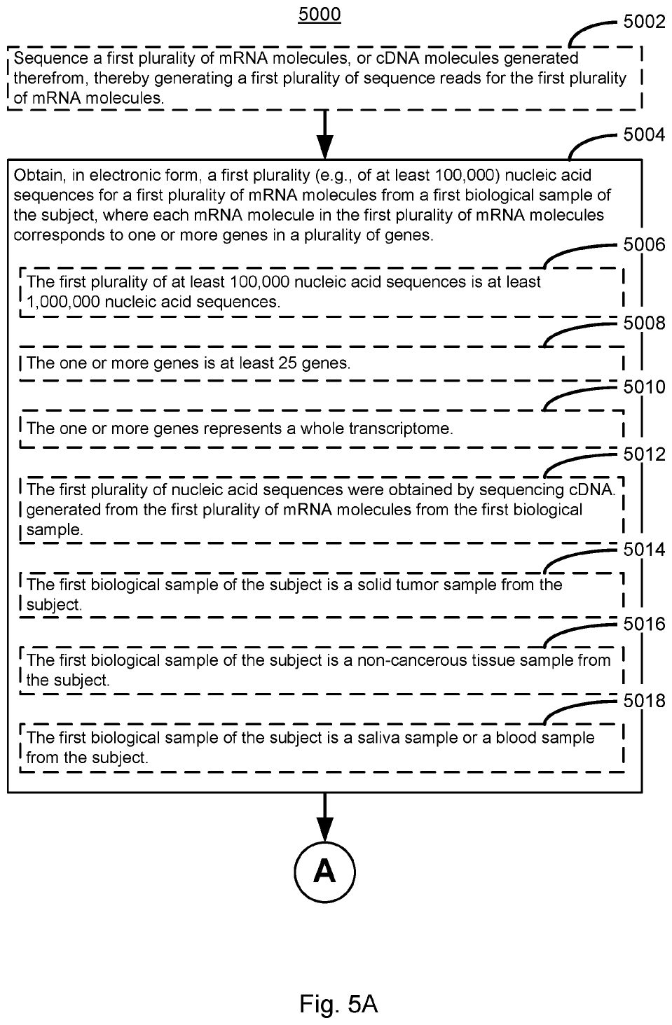

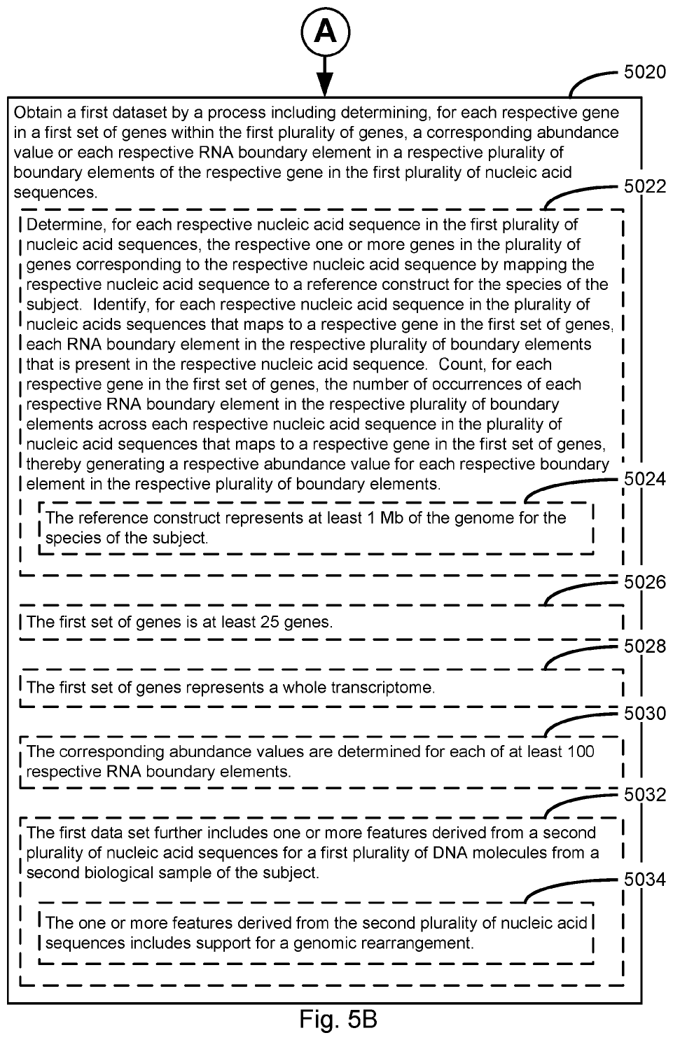

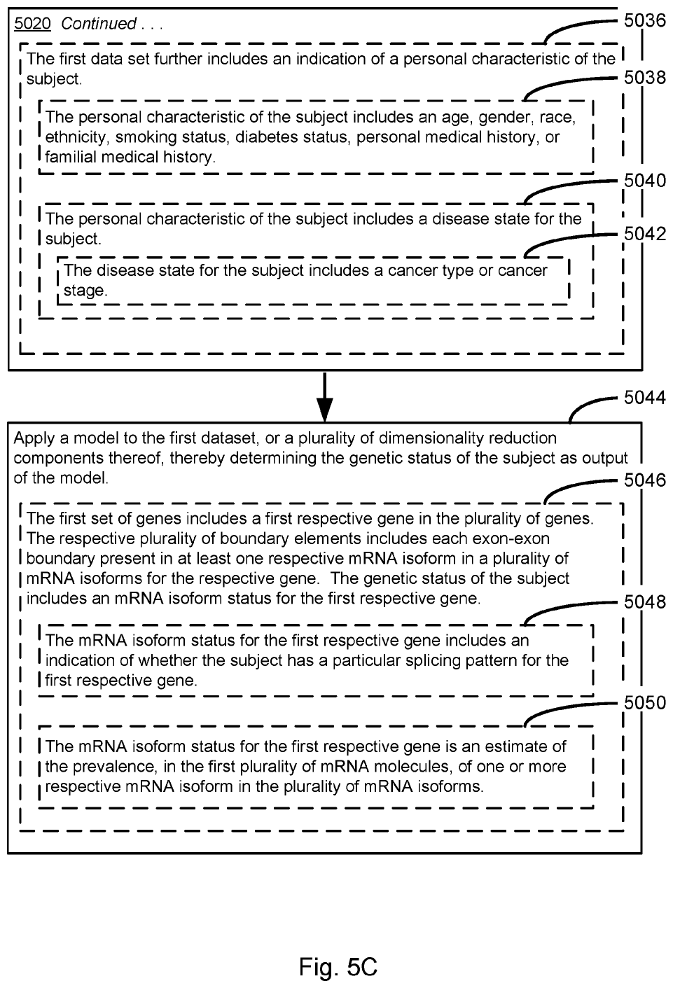

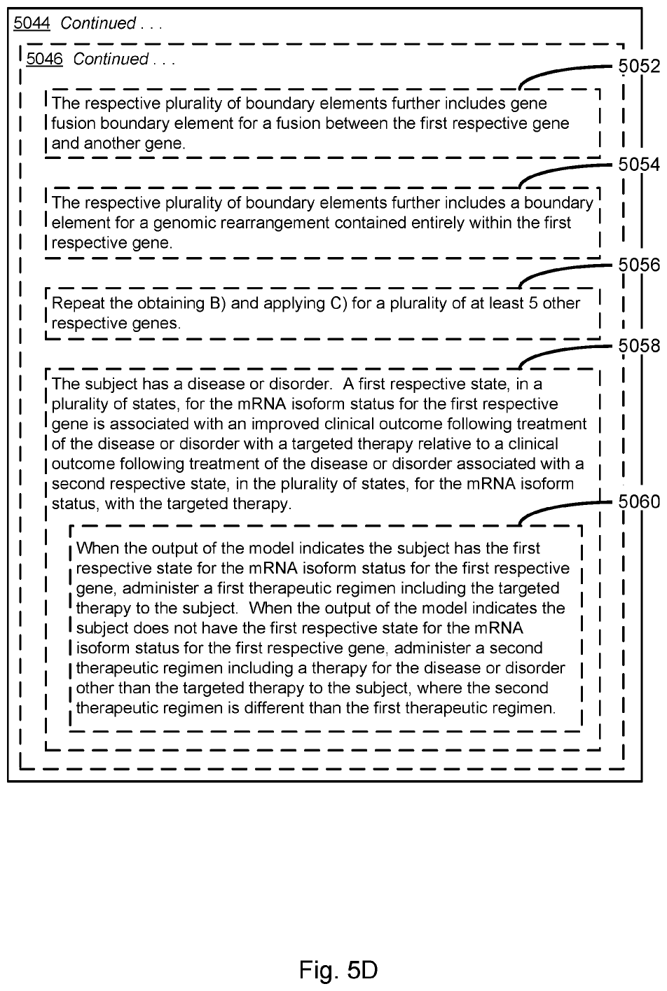

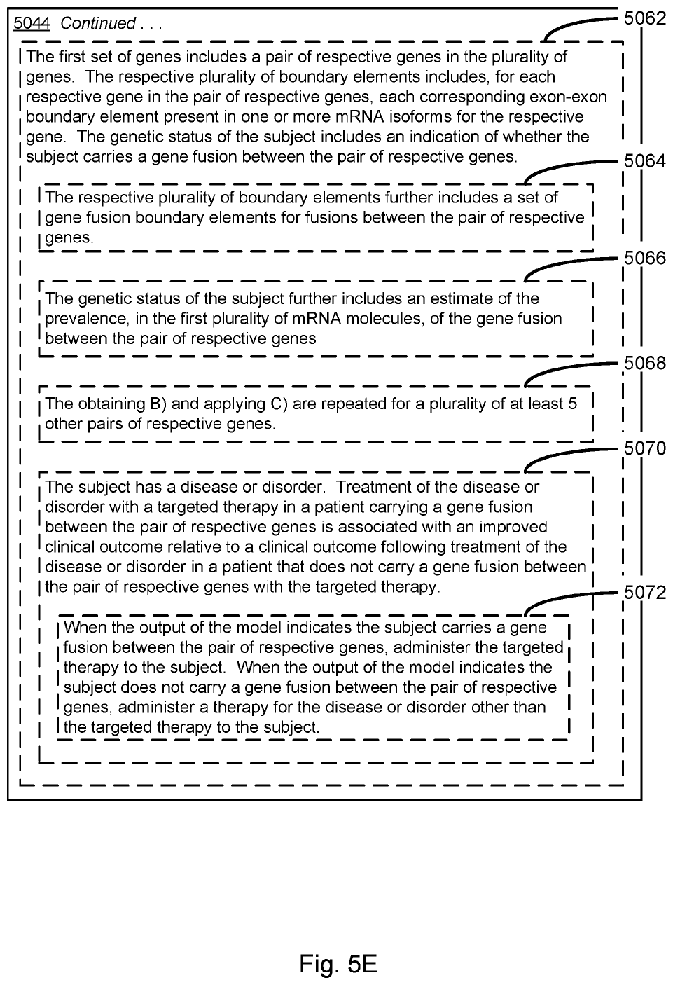

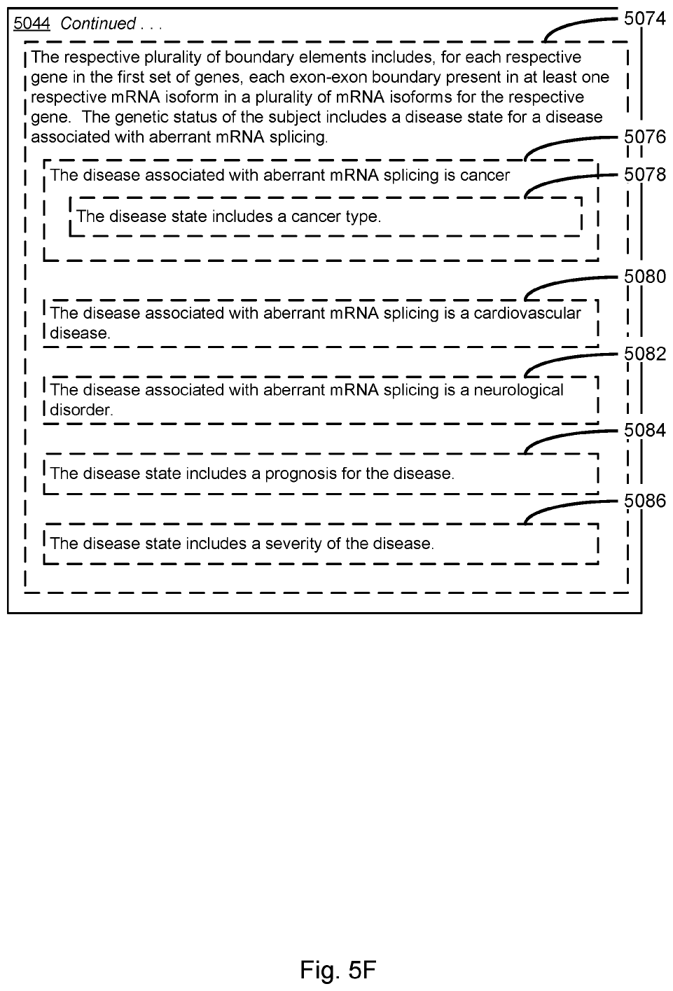

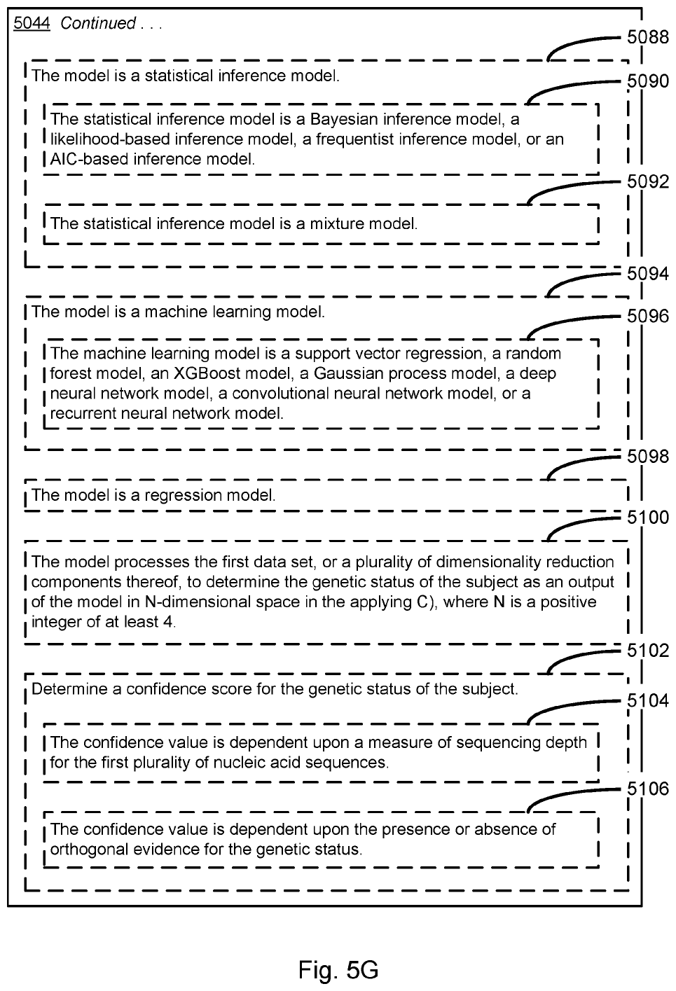

An overview of methods for providing clinical support for personalized therapy is described above with reference to FIGS. 1-4F above. Below, systems and methods based on RNA boundary analysis are described for improving determination of genetic statuses, e.g., within the context of the methods and systems described above, are described with reference to FIGS. 4B and 5A-5G.

Many of the embodiments described below, in conjunction with FIGS. 4B and 5A-5G, relate to analyses performed using sequencing data for nucleic acid molecules obtained from samples of a subject. Generally, these embodiments are independent and, thus, not reliant upon any particular nucleic acid sequencing methods. However, in some embodiments, the methods described below include generating the sequencing data.

In one aspect, the disclosure provides a method 5000 for determining a genetic status of a subject. In some embodiments, all or part of the method is performed on a computer system having one or more processors and memory storing one or more programs for execution by the one or more processors, e.g., such as system 100.

In some embodiments, the method includes sequencing (5002) a first plurality of mRNA molecules from a sample of the subject, or cDNA molecules generated therefrom, thereby generating a first plurality of sequence reads for the first plurality of mRNA molecules. However, in some embodiments, the sequencing has already been performed, e.g., and the method begins with obtaining nucleic acid sequences from the previously executed sequencing reaction.

In some embodiments, the sequencing reaction is a panel-targeted sequencing reaction that uses a plurality of nucleic acid capture probe species. In some embodiments, each respective nucleic acid probe species (e.g., all nucleic acid probes that align to the same subsequence of a respective target region) in the plurality of nucleic acid probe species aligns to a different subsequence of a respective target region of a reference construct for the species of the subject. For instance, in some embodiments, a first respective set of nucleic acid probes tiles (e.g., via overlapping or non-overlapping tiling) a respective genomic region, such as a gene. Thus, the nucleic acid probes in the set of probes bind to different subsequences of the genomic region.

As used herein, a “nucleic acid probe species” refers to all nucleic acid probes in a composition that align to the same or substantially the same genomic sequence (e.g., the first 150 nucleotides of a particular exon of a gene). Generally, all probes of a particular nucleic acid probe species will have the same nucleotide sequence. However, in some embodiments, a particular probe of nucleic acid probe species may have one or a small number of nucleotide variations relative to other probes within the nucleic acid probe species. Regardless, two probes that differ by one or a small number of nucleotide variants still belong to the same nucleic acid probe species because they align to the same position in the genome. Similarly, it can be envisioned that, in some embodiments, a probe in a particular nucleic acid probe species may be one or a small number of nucleotides longer or shorter than other probes in the particular nucleic acid probe species. Furthermore, it can be envisioned that, in some embodiments, a probe in a particular nucleic acid probe species may be shifted by one or a small number of nucleotides relative to the sequence of other probes in the particular nucleic acid probe species. In addition, probes in a particular nucleic acid probe species may be differently conjugated to a chemical moiety.

In some embodiments, the plurality of nucleic acid probe species comprises at least 100, at least 500, at least 1000, at least 2000, at least 5000, at least 10,000, at least 50,000, at least 100,000, at least 500,000, at least 1,000,000, at least 2,500,000, or at least 5,000,000 nucleic acid probe species. In some embodiments, the plurality of nucleic acid probe species is no more than 10,000,000, no more than 1,000,000, no more than 500,000, no more than 100,000, no more than 50,000, no more than 10,000, no more than 5000, no more than 1000, or no more than 500 nucleic acid probe species. In some embodiments, the plurality of nucleic acid probe species is from 100 to 500, from 250 to 1000, from 1000 to 5000, from 1000 to 10,000,000, from 1,000,000 to 10,000,000, from 100 to 5,000,000, or from 100,000 to 500,000 nucleic acid probe species. In some embodiments, the plurality of nucleic acid probe species falls within another range starting no lower than 100 nucleic acid probe species and ending no higher than 10,000,000 nucleic acid probe species.

Additional embodiments for probes suitable for use in the present disclosure are further described in U.S. patent application Ser. No. 17/076,704, filed Oct. 21, 2020, the content of which is hereby incorporated by reference, in its entirety, for all purposes.

Method 5000 includes obtaining (5004), in electronic form, a first plurality (e.g., of at least 100,000) nucleic acid sequences for a first plurality of mRNA molecules from a first biological sample of the subject, where each mRNA molecule in the first plurality of mRNA molecules corresponds to one or more genes in a plurality of genes.

In some embodiments, the first plurality nucleic acid sequences is at least 1,000,000 sequences (5006). In some embodiments, the first plurality of nucleic acid sequences is at least 5000 nucleic acid sequences, at least 10,000 nucleic acid sequences, at least 50,000 nucleic acid sequences, at least 100,000 nucleic acid sequences, at least 250,000 nucleic acid sequences, at least 500,000 nucleic acid sequences, at least 2,000,000 nucleic acid sequences, or more nucleic acid sequences. In some embodiments, the first plurality of nucleic acid sequences is no more than 10,000,000 nucleic acid sequences, no more than 5,000,000 nucleic acid sequences, no more than 2,500,000 nucleic acid sequences, no more than 1,000,000 nucleic acid sequences, nor more than 500,000 nucleic acid sequences, no more than 250,000 nucleic acid sequences, or less.

In some embodiments, the first plurality nucleic acid sequences is from 5000 to 10,000,000 nucleic acid sequences. In some embodiments, the first plurality nucleic acid sequences is from 5000 to 5,000,000 nucleic acid sequences. In some embodiments, the first plurality nucleic acid sequences is from 5000 to 2,500,000 nucleic acid sequences. In some embodiments, the first plurality nucleic acid sequences is from 5000 to 1,000,000 nucleic acid sequences. In some embodiments, the first plurality nucleic acid sequences is from 5000 to 500,000 nucleic acid sequences. In some embodiments, the first plurality nucleic acid sequences is from 5000 to 250,000 nucleic acid sequences. In some embodiments, the first plurality nucleic acid sequences is from 25,000 to 10,000,000 nucleic acid sequences. In some embodiments, the first plurality nucleic acid sequences is from 25,000 to 5,000,000 nucleic acid sequences. In some embodiments, the first plurality nucleic acid sequences is from 25,000 to 2,500,000 nucleic acid sequences. In some embodiments, the first plurality nucleic acid sequences is from 25,000 to 5,000,000 nucleic acid sequences. In some embodiments, the first plurality nucleic acid sequences is from 25,000 to 2,500,000 nucleic acid sequences. In some embodiments, the first plurality nucleic acid sequences is from 25,000 to 1,000,000 nucleic acid sequences. In some embodiments, the first plurality nucleic acid sequences is from 25,000 to 500,000 nucleic acid sequences. In some embodiments, the first plurality nucleic acid sequences is from 25,000 to 250,000 nucleic acid sequences. In some embodiments, the first plurality nucleic acid sequences is from 100,000 to 10,000,000 nucleic acid sequences. In some embodiments, the first plurality nucleic acid sequences is from 100,000 to 5,000,000 nucleic acid sequences. In some embodiments, the first plurality nucleic acid sequences is from 100,000 to 2,500,000 nucleic acid sequences. In some embodiments, the first plurality nucleic acid sequences is from 100,000 to 1,000,000 nucleic acid sequences. In some embodiments, the first plurality nucleic acid sequences is from 100,000 to 500,000 nucleic acid sequences. In some embodiments, the first plurality nucleic acid sequences is from 100,000 to 250,000 nucleic acid sequences. In some embodiments, the first plurality nucleic acid sequences is from 250,000 to 10,000,000 nucleic acid sequences. In some embodiments, the first plurality nucleic acid sequences is from 250,000 to 5,000,000 nucleic acid sequences. In some embodiments, the first plurality nucleic acid sequences is from 250,000 to 2,500,000 nucleic acid sequences. In some embodiments, the first plurality nucleic acid sequences is from 250,000 to 1,000,000 nucleic acid sequences. In some embodiments, the first plurality nucleic acid sequences is from 250,000 to 500,000 nucleic acid sequences.

In some embodiments, the one or more genes is at least 25 genes (5008). In some embodiments, the one or more genes is at least 10, at least 15, at least 25, at least 30, at least 40, at least 50, at least 100, at least 200, at least 250, at least 400, at least 500, at least 600, at least 700, at least 800, at least 900, at least 1000, at least 2000, at least 2500, at least 4000, at least 5000, at least 6000, at least 7000, at least 8000, at least 9000, at least 10,000, at least 15,000, or at least 20,000 genes. In some embodiments, the one or more genes is no more than 40,000, no more than 30,000, no more than 20,000, no more than 10,000, no more than 8000, no more than 7500, no more than 5000, no more than 4000, no more than 3000, no more than 2000, no more than 1000, no more than 750, no more than 500, no more than 250, no more than 100, no more than 50, or no more than 25 genes. In some embodiments, the one or more genes is from 10 to 50,000 genes. In some embodiments, the one or more genes is from 10 to 40,000 genes. In some embodiments, the one or more genes is from 10 to 25,000 genes. In some embodiments, the one or more genes is from 10 to 10,000 genes. In some embodiments, the one or more genes is from 10 to 5000 genes. In some embodiments, the one or more genes is from 10 to 1000 genes. In some embodiments, the one or more genes is from 10 to 500 genes. In some embodiments, the one or more genes is from 25 to 50,000 genes. In some embodiments, the one or more genes is from 25 to 40,000 genes. In some embodiments, the one or more genes is from 25 to 25,000 genes. In some embodiments, the one or more genes is from 25 to 10,000 genes. In some embodiments, the one or more genes is from 25 to 5000 genes. In some embodiments, the one or more genes is from 25 to 1000 genes. In some embodiments, the one or more genes is from 25 to 500 genes. In some embodiments, the one or more genes is from 100 to 50,000 genes. In some embodiments, the one or more genes is from 100 to 40,000 genes. In some embodiments, the one or more genes is from 100 to 25,000 genes. In some embodiments, the one or more genes is from 100 to 10,000 genes. In some embodiments, the one or more genes is from 100 to 5000 genes. In some embodiments, the one or more genes is from 100 to 1000 genes. In some embodiments, the one or more genes is from 100 to 500 genes. In some embodiments, the one or more genes is from 500 to 50,000 genes. In some embodiments, the one or more genes is from 500 to 40,000 genes. In some embodiments, the one or more genes is from 500 to 25,000 genes. In some embodiments, the one or more genes is from 500 to 10,000 genes. In some embodiments, the one or more genes is from 500 to 5000 genes. In some embodiments, the one or more genes is from 500 to 1000 genes. In some embodiments, the one or more genes is from 1000 to 50,000 genes. In some embodiments, the one or more genes is from 1000 to 40,000 genes. In some embodiments, the one or more genes is from 1000 to 25,000 genes. In some embodiments, the one or more genes is from 1000 to 10,000 genes. In some embodiments, the one or more genes is from 1000 to 5000 genes. In some embodiments, the one or more genes is from 5000 to 50,000 genes. In some embodiments, the one or more genes is from 5000 to 40,000 genes. In some embodiments, the one or more genes is from 5000 to 25,000 genes. In some embodiments, the one or more genes is from 5000 to 10,000 genes. In some embodiments, the one or more genes represents a whole transcriptome (5010).

In some embodiments, first plurality of nucleic acid sequences were obtained by sequencing cDNA. generated from the first plurality of mRNA molecules from the first biological sample (5012).

In some embodiments, the first biological sample of the subject is a solid tumor sample from the subject (5014). In some embodiments, the first biological sample of the subject is a non-cancerous tissue sample from the subject (5016). In some embodiments, the first biological sample of the subject is a saliva sample or a blood sample from the subject (5018). In some embodiments, the method is performed for more than one type of biological sample from the subject, e.g., for 2, 3, 4, 5, 6, 7, 8, 9, 10, or more biological samples from the subject.

Method 5000 also includes obtaining (5020) a first dataset by a process including determining, for each respective gene (e.g., gene 602a illustrated in FIG. 6) in a first set of genes within the first plurality of genes, a corresponding abundance value (e.g., abundance values 612 illustrated in FIG. 6) for each respective RNA boundary element (e.g., e.g., boundary elements 606 illustrated in FIG. 6) in a respective plurality of boundary elements of the respective gene in the first plurality of nucleic acid sequences. For example, in some embodiments, the abundance value is a value for the number of unique occurrences of the RNA boundary sub-sequence in the plurality of sequences. In some embodiments, this is performed by identifying instances of the sequence of a respective boundary element in the first plurality of nucleic acid sequences and/or sequences known to flank both sides of a boundary element in the same respective nucleic acid sequence in the plurality of nucleic acid sequences. In some embodiments, the plurality of boundary elements of the respective gene is each possible exon-exon boundary of the gene. In some embodiments, abundances for one or more gene fusion boundaries are also determined.

In some embodiments, the process for obtaining the first dataset includes determining (5022), for each respective nucleic acid sequence in the first plurality of nucleic acid sequences, the respective one or more genes in the plurality of genes corresponding to the respective nucleic acid sequence by mapping the respective nucleic acid sequence to a reference construct for the species of the subject, identifying, for each respective nucleic acid sequence in the plurality of nucleic acids sequences that maps to a respective gene in the first set of genes, each RNA boundary element in the respective plurality of boundary elements that is present in the respective nucleic acid sequence, and counting, for each respective gene in the first set of genes, the number of occurrences of each respective RNA boundary element in the respective plurality of boundary elements across each respective nucleic acid sequence in the plurality of nucleic acid sequences that maps to a respective gene in the first set of genes, thereby generating a respective abundance value for each respective boundary element in the respective plurality of boundary elements.

In some embodiments, the reference construct represents at least 1 Mb of the genome, exome, and/or transcriptome for the species of the subject (5024). In other embodiments, the reference construct represents at least 250 kb, 500 kb, 750 kb, 1 Mb, 2 Mb, 5 Mb, 10 Mb, 25 Mb, 50 Mb, 100 Mb, 250 Mb, or more of the genome, exome, and/or transcriptome for the species of the subject. However, in some embodiments, there is no size limitation of the reference sequence. For example, in some embodiments, the reference sequence can be a sequence for a single locus, e.g., a single exon, gene, etc. within the genome, exome, and/or transcriptome for the species of the subject.

In some embodiments, the first set of genes is a single gene. In some embodiments, the first set of genes is at least 25 genes (5026). In some embodiments, the first set of genes is at least 10, at least 15, at least 25, at least 30, at least 40, at least 50, at least 100, at least 200, at least 250, at least 400, at least 500, at least 600, at least 700, at least 800, at least 900, at least 1000, at least 2000, at least 2500, at least 4000, at least 5000, at least 6000, at least 7000, at least 8000, at least 9000, at least 10,000, at least 15,000, or at least 20,000 genes. In some embodiments, the first set of genes is no more than 40,000, no more than 30,000, no more than 20,000, no more than 10,000, no more than 8000, no more than 7500, no more than 5000, no more than 4000, no more than 3000, no more than 2000, no more than 1000, no more than 750, no more than 500, no more than 250, no more than 100, no more than 50, or no more than 25 genes. In some embodiments, the first set of genes is from 10 to 50,000 genes. In some embodiments, the first set of genes is from 10 to 40,000 genes. In some embodiments, the first set of genes is from 10 to 25,000 genes. In some embodiments, the first set of genes is from 10 to 10,000 genes. In some embodiments, the first set of genes is from 10 to 5000 genes. In some embodiments, the first set of genes is from 10 to 1000 genes. In some embodiments, the first set of genes is from 10 to 500 genes. In some embodiments, the first set of genes is from 25 to 50,000 genes. In some embodiments, the first set of genes is from 25 to 40,000 genes. In some embodiments, the first set of genes is from 25 to 25,000 genes. In some embodiments, the first set of genes is from 25 to 10,000 genes. In some embodiments, the first set of genes is from 25 to 5000 genes. In some embodiments, the first set of genes is from 25 to 1000 genes. In some embodiments, the first set of genes is from 25 to 500 genes. In some embodiments, the first set of genes is from 100 to 50,000 genes. In some embodiments, the first set of genes is from 100 to 40,000 genes. In some embodiments, the first set of genes is from 100 to 25,000 genes. In some embodiments, the first set of genes is from 100 to 10,000 genes. In some embodiments, the first set of genes is from 100 to 5000 genes. In some embodiments, the first set of genes is from 100 to 1000 genes. In some embodiments, the first set of genes is from 100 to 500 genes. In some embodiments, the first set of genes is from 500 to 50,000 genes. In some embodiments, the first set of genes is from 500 to 40,000 genes. In some embodiments, the first set of genes is from 500 to 25,000 genes. In some embodiments, the first set of genes is from 500 to 10,000 genes. In some embodiments, the first set of genes is from 500 to 5000 genes. In some embodiments, the first set of genes is from 500 to 1000 genes. In some embodiments, the first set of genes is from 1000 to 50,000 genes. In some embodiments, the first set of genes is from 1000 to 40,000 genes. In some embodiments, the first set of genes is from 1000 to 25,000 genes. In some embodiments, the first set of genes is from 1000 to 10,000 genes. In some embodiments, the first set of genes is from 1000 to 5000 genes. In some embodiments, the first set of genes is from 5000 to 50,000 genes. In some embodiments, the first set of genes is from 5000 to 40,000 genes. In some embodiments, the first set of genes is from 5000 to 25,000 genes. In some embodiments, the first set of genes is from 5000 to 10,000 genes. In some embodiments, the first set of genes represents a whole transcriptome (5028).

In some embodiments, corresponding abundance values are determined for each of at least 100 respective RNA boundary elements (5030). In some embodiments, corresponding abundance values are determined for a first set of RNA boundary elements. In some embodiments, the first set of RNA boundary elements is at least 5, at least 10, at least 15, at least 25, at least 30, at least 40, at least 50, at least 100, at least 200, at least 250, at least 400, at least 500, at least 600, at least 700, at least 800, at least 900, at least 1000, at least 2000, at least 2500, at least 4000, at least 5000, at least 6000, at least 7000, at least 8000, at least 9000, at least 10,000, at least 15,000, at least 20,000, at least 50,000, at least 100,000, at least 250,000, at least 500,000, at least 1,000,000, or more RNA boundary elements. In some embodiments, the first set of RNA boundary elements is no more than 10,000,000, no more than 5,000,000, no more than 2,500,000, no more than 1,000,000, no more than 750,000, no more than 500,000, no more than 250,000, no more than 100,000, no more than 50,000, no more than 25,000, no more than 10,000, no more than 5000, or no more than 2500 RNA boundary elements. In some embodiments, the first set of RNA boundary elements is from 10 to 10,000,000 RNA boundary elements. In some embodiments, the first set of RNA boundary elements is from 10 to 5,000,000 RNA boundary elements. In some embodiments, the first set of RNA boundary elements is from 10 to 2,500,000 RNA boundary elements. In some embodiments, the first set of RNA boundary elements is from 10 to 1,000,000 RNA boundary elements. In some embodiments, the first set of RNA boundary elements is from 10 to 500,000 RNA boundary elements. In some embodiments, the first set of RNA boundary elements is from 10 to 100,000 RNA boundary elements. In some embodiments, the first set of RNA boundary elements is from 10 to 50,000 RNA boundary elements. In some embodiments, the first set of RNA boundary elements represents a whole transcriptome.

In some embodiments, the first data set further includes one or more features derived from a second plurality of nucleic acid sequences for a first plurality of DNA molecules from a second biological sample of the subject (5032). In some embodiments, the one or more features derived from the second plurality of nucleic acid sequences includes support for a genomic rearrangement (5034). For example, in some embodiments, the one or more features derived from the second plurality of nucleic acid sequences includes evidence (e.g., direct or indirect) of a boundary element in the genome of the subject, e.g., where the second plurality of nucleic acid sequences are for genomic DNA from the subject.

In some embodiments, the first data set further includes an indication of a personal characteristic of the subject (5036). In some embodiments, the personal characteristic of the subject includes an age, gender, race, ethnicity, smoking status, diabetes status, personal medical history, or familial medical history of the subject (5038).

In some embodiments, the personal characteristic of the subject includes a disease state for the subject (5040). In some embodiments, the disease state for the subject includes a cancer type or cancer stage (5044).

Method 5000 also includes applying (5044) a model to the first dataset, or a plurality of dimensionality reduction components thereof, thereby determining the genetic status of the subject as output of the model.

A variety of dimensionality reduction techniques can be used. Examples include, but are not limited to, principal component analysis (PCA), non-negative matrix factorization (NMF), linear discriminant analysis (LDA), diffusion maps, or network (e.g., neural network) techniques such as an autoencoder.

In some embodiments, the dimension reduction is a principal components algorithm, a random projection algorithm, an independent component analysis algorithm, a feature selection method, a factor analysis algorithm, Sammon mapping, curvilinear components analysis, a stochastic neighbor embedding (SNE) algorithm, an Isomap algorithm, a maximum variance unfolding algorithm, a locally linear embedding algorithm, a t-SNE algorithm, a non-negative matrix factorization algorithm, a kernel principal component analysis algorithm, a graph-based kernel principal component analysis algorithm, a linear discriminant analysis algorithm, a generalized discriminant analysis algorithm, a uniform manifold approximation and projection (UMAP) algorithm, a LargeVis algorithm, a Laplacian Eigenmap algorithm, or a Fisher's linear discriminant analysis algorithm. See, for example, Fodor, 2002, “A survey of dimension reduction techniques,” Center for Applied Scientific Computing, Lawrence Livermore National, Technical Report UCRL-ID-148494; Cunningham, 2007, “Dimension Reduction,” University College Dublin, Technical Report UCD-CSI-2007-7, Zahorian et al., 2011, “Nonlinear Dimensionality Reduction Methods for Use with Automatic Speech Recognition,” Speech Technologies. doi:10.5772/16863. ISBN 978-953-307-996-7; and Lakshmi et al., 2016, “2016 IEEE 6th International Conference on Advanced Computing (IACC),” pp. 31-34. doi:10.1109/IACC.2016.16, ISBN 978-1-4673-8286-1, the contents of which are hereby incorporated by reference, in their entireties, for all purposes. Accordingly, in some embodiments, the dimension reduction is a principal component analysis (PCA) algorithm, and each respective extracted dimension reduction component comprises a respective principal component derived by the PCA. In such embodiments, the number of principal components in the plurality of principal components can be limited to a threshold number of principal components calculated by the PCA algorithm. The threshold number of principal components can be, for example, at least 5, at least 10, at least 20, at least 50, at least 100, at least 1000, at least 1500, or any other number.

In some embodiments, the method further includes performing manifold learning using the dataset. Generally, manifold learning is used to describe the low-dimensional structure of high-dimensional data by determining maximal variations in a dataset. Examples include, but are not limited to, force-directed layout (see, e.g., Fruchterman, T. M., & Reingold, E. M. (1991). Graph drawing by force-directed placement. Software: Practice and experience, 21(11), 1129-1164) (e.g., Force Atlas 2), t-distributed stochastic neighbor embedding (t-SNE), locally linear embedding (see, e.g., Roweis, S. T., & Saul, L. K. (2000). Nonlinear dimensionality reduction by locally linear embedding. Science, 290(5500), 2323-2326), local linear isometric mapping (ISOMAP; see, e.g., Tenenbaum, J. B., De Silva, V., & Langford, J. C. (2000). A global geometric framework for nonlinear dimensionality reduction. Science, 290(5500), 2319-2323), kernel PCA, graph-based kernel PCA, Potential of Heat-Diffusion for Affinity Based Trajectory Embedding (PHATE), generalized discriminant analysis (GDA), Uniform Manifold Approximation and Projection (UMAP), or kernel discriminant analysis. In some embodiments, the method includes performing discriminant analysis. Force-directed layouts are useful in various particular embodiments because of their ability to identify new, lower dimensions that encode non-linear aspects of the underlying data which arise from underlying relationships between data elements. Force directed layouts use physics-based models as mechanisms for determining a reduced dimensionality that best represents the data. As an example, a force directed layout uses a form of physics simulation in which, in this embodiment, each input element in the first and/or second mapped datasets is assigned a “repulsion” force and there exists a global “gravitation force” that, when computed over the plurality of elements, identifies sectors of the data that “diffuse” together under these competing “forces.” Force directed layouts make few assumptions about the structure of the data, and do not impose a de-noising approach. Manifold learning is further described, for example, in Wang et al., 2004, “Adaptive Manifold Learning,” Advances in Neural Information Processing Systems 17, the content of which is hereby incorporated by reference, in its entirety, for all purposes.

In some embodiments, first set of genes includes a first respective gene in the plurality of genes (5046). The respective plurality of boundary elements includes each exon-exon boundary present in at least one respective mRNA isoform in a plurality of mRNA isoforms for the respective gene, e.g., for each respective mRNA isoform in a plurality of mRNA isoforms for the respective gene, each corresponding exon-exon boundary element present in the respective mRNA isoform. The genetic status of the subject includes an mRNA isoform status for the first respective gene, e.g., the presence of a particular mRNA isoform, the absence of a particular mRNA isoform, a prevalence of a particular mRNA isoform, a prevalence of a first respective mRNA isoform relative to a prevalence of a second respective mRNA isoform, the number of detected isoforms and percentage prevalence for each, how that profile compares to normal splicing patterns seen in a patient population, etc. In some embodiments, the mRNA isoform status for the first respective gene includes an indication (e.g., a probability, likelihood, dichotomous or binary prediction) of whether the subject has a particular splicing pattern for the first respective gene (5048). In some embodiments, the mRNA isoform status for the first respective gene is an estimate of the prevalence (e.g., prevalence relative to total mRNA or to one or more other mRNA isoforms), in the first plurality of mRNA molecules, of one or more respective mRNA isoforms in the plurality of mRNA isoforms (5050). In some embodiments, the respective plurality of boundary elements further includes gene fusion boundary element for a fusion between the first respective gene and another gene (5052). In some embodiments, the respective plurality of boundary elements further includes a boundary element for a genomic rearrangement (e.g., insertion, deletion, or inversion) contained entirely within the first respective gene (5054).

In some embodiments, the method is repeated for a plurality of at least 5 other respective genes. In some embodiments, the method is repeated for at least 1, 2, 3, 4, 5, 6, 7, 8, 9, 10, 20, 30, 40, 50, 60, 70, 80, 90, 100, 250, 500, 1000, 2500, 5000, 10,000, or more genes.

In some embodiments, the subject has a disease or disorder (5058). A first respective state, in a plurality of states, for the mRNA isoform status for the first respective gene is associated with an improved clinical outcome following treatment of the disease or disorder with a targeted therapy relative to a clinical outcome following treatment of the disease or disorder associated with a second respective state, in the plurality of states, for the mRNA isoform status, with the targeted therapy. In some embodiments, when the output of the model indicates the subject has the first respective state for the mRNA isoform status for the first respective gene, the method includes administering a first therapeutic regimen including the targeted therapy to the subject, and when the output of the model indicates the subject does not have the first respective state for the mRNA isoform status for the first respective gene, the method includes administering a second therapeutic regimen including a therapy for the disease or disorder other than the targeted therapy to the subject, where the second therapeutic regimen is different than the first therapeutic regimen (5060).

In some embodiments of the methods described herein, the treatment is selected from the group consisting of spliceostatin A, pladienolide-B, GEX1A, E1707, Amiloride, H3B-8800, splice-switching antisense oligonucleotides (SSO), anti-sense oligonucleotides (ASO), short hairpin RNA interference/small interference RNA, clustered regularly interspaced short palindromic repeats (CRISPR)-associated (Cas) systems, CRISPR-Cas13a enzyme, and single-base editors (BEs), cytosine-BEs (CBEs) and adenosine-BEs (ABEs). In some embodiments of the methods described herein, the treatment is selected from inhibitors of the EGFR (Epidermal Growth Factor Receptor), MET (Mesenchymal Epithelial Transition Factor), and AR (Androgen Receptor) genes. Additional embodiments involved methods where the EGFR inhibitor is a tyrosine kinase inhibitor selected from the group consisting of osimertinib, rociletinib, olmutinib, nazartinib, naquotinib, mavelertinib (PF-0647775), and avitinib or an anti-EGFR antibody selected from the group consisting of cetuximab, panitumumab, nimotuzumab, and necitumumab. In some embodiments of the methods described herein, the treatment is a MET inhibitor is selected from the group consisting of crizotinib, tivantinib, savolitinib, tepotinib, cabozantinib, and foretinib or an anti-MET antibody selected from ficlatuzumab and rilotumumab. In some embodiments of the methods described herein, the treatment is an androgen receptor antagonist selected from the group consisting of flutamide, bicalutamide, and nilutamide. The method can also be done where the disease is a thalassemia, familial dysautonomia, spinal muscular atrophy, amyotrophic lateral sclerosis, or Parkinson's disease.

In some embodiments, the first set of genes includes a pair of respective genes in the plurality of genes (5062), e.g., two genes known to rearrange with each other to form a gene fusion. The respective plurality of boundary elements includes, for each respective gene in the pair of respective genes, each corresponding exon-exon boundary element present in one or more mRNA isoforms for the respective gene. The genetic status of the subject includes an indication (e.g., a probability, likelihood, dichotomous or binary prediction) of whether the subject carries a gene fusion between the pair of respective genes. In some embodiments, the respective plurality of boundary elements further includes a set of gene fusion boundary elements for fusions between the pair of respective genes (5064). In some embodiments, the genetic status of the subject further includes an estimate of the prevalence, in the first plurality of mRNA molecules, of the gene fusion between the pair of respective genes (5066).

In some embodiments, the method is repeated for a plurality of at least 5 other pairs of respective genes (5068). In some embodiments, the method is repeated for at least 1, 2, 3, 4, 5, 6, 7, 8, 9, 10, 20, 30, 40, 50, 60, 70, 80, 90, 100, 250, 500, 1000, 2500, 5000, 10,000, or more pairs of respective genes.

In some embodiments, the subject has a disease or disorder (5070). Treatment of the disease or disorder with a targeted therapy in a patient carrying a gene fusion between the pair of respective genes is associated with an improved clinical outcome relative to a clinical outcome following treatment of the disease or disorder in a patient that does not carry a gene fusion between the pair of respective genes with the targeted therapy. In some embodiments, when the output of the model indicates the subject carries a gene fusion between the pair of respective genes, the method includes administering the targeted therapy to the subject, and when the output of the model indicates the subject does not carry a gene fusion between the pair of respective genes, administer a therapy for the disease or disorder other than the targeted therapy to the subject (5072).

In some embodiments, the respective plurality of boundary elements includes, for each respective gene in the first set of genes, each exon-exon boundary present in at least one respective mRNA isoform in a plurality of mRNA isoforms for the respective gene (5074). The genetic status of the subject includes a disease state for a disease associated with aberrant mRNA splicing. In some embodiments, the disease associated with aberrant mRNA splicing is cancer (5076). In some embodiments, the disease state includes a cancer type (5078). In some embodiments, the disease associated with aberrant mRNA splicing is a cardiovascular disease (5080). In some embodiments, the disease associated with aberrant mRNA splicing is a neurological disorder (5082). In some embodiments, the disease state includes a prognosis for the disease (5084), e.g., a prediction for development of the disease, a prediction for survival, a prediction for therapeutic efficacy, a prediction for disease-free survival, a prediction for recurrence, etc. In some embodiments, the disease state includes a severity of the disease (5086), e.g., a cancer stage.

In certain embodiments of the present method the cancer is selected from group consisting breast cancer, squamous cell cancer, lung cancer, adenocarcinoma of the lung, and squamous carcinoma of the lung, head and neck cancer, cancer of the peritoneum, hepatocellular cancer, gastric cancer, stomach cancer, pancreatic cancer, ovarian cancer, cervical cancer, liver cancer, bladder cancer, hepatoma, colon cancer, colorectal cancer, endometrial or uterine carcinoma, salivary gland carcinoma, kidney or renal cancer, liver cancer, prostate cancer, vulval cancer, thyroid cancer, and hepatic carcinoma, as well as B-cell lymphoma, chronic lymphocytic leukemia (CLL), acute lymphoblastic leukemia (ALL), hairy cell leukemia, chronic myeloblastic leukemia, and post-transplant lymphoproliferative disorder (PTLD). In other embodiments of the present method the cancer is selected from the subgroups of small-cell lung cancer, non-small cell lung cancer (NSCLC), adenocarcinoma of the lung, and squamous carcinoma of the lung, squamous NSCLC, low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's Macroglobulinemia, breast cancer subtype Luminal A (hormone receptor (HR)+/human epidermal growth factor receptor (HER2)−); breast cancer subtype Luminal B (HR+/HER2+); breast cancer subtype Triple-negative or (HR−/HER2−); breast cancer subtype HER2 positive; and prostate cancer subtypes involving changes in the ERG, ETV1/4, and FLI1 genes and prostate cancer subtypes defined by mutations in FOXA1, SPOP, and IDH1 genes. In some embodiments, the disease or disorder is a thalassemia, familial dysautonomia, spinal muscular atrophy, amyotrophic lateral sclerosis, or Parkinson's disease.

In some embodiments, the model is a statistical inference model (5088). In some embodiments, the statistical inference model is a Bayesian inference model, a likelihood-based inference model, a frequentist inference model, or an AIC-based inference model (5090). In some embodiments, the statistical inference model is a mixture model (5092). In some embodiments, the statistical inference comprises any suitable statistical inference model disclosed herein, and/or any combinations, modifications, substitutions, additions, or deletions thereof as will be apparent to one skilled in the art (see, e.g., the section entitled, “Definitions: Classifier,” above).

In some embodiments, the model is a machine learning model (5094). In some embodiments, the machine learning model is a support vector regression, a random forest model, an XGBoost model, a Gaussian process model, a deep neural network model, a convolutional neural network model, or a recurrent neural network model (5096). In some embodiments, the machine learning model comprises any suitable machine learning model disclosed herein, and/or any combinations, modifications, substitutions, additions, or deletions thereof as will be apparent to one skilled in the art (see, e.g., the section entitled, “Definitions: Classifier,” above).

In some embodiments, the model is a regression model (5098). In some embodiments, the regression model comprises any suitable regression model disclosed herein, and/or any combinations, modifications, substitutions, additions, or deletions thereof as will be apparent to one skilled in the art (see, e.g., the section entitled, “Definitions: Classifier,” above).

In some embodiments, the model processes the first data set, or a plurality of dimensionality reduction components thereof, to determine the genetic status of the subject as an output of the model in N-dimensional space in the applying (5100), where N is a positive integer of at least 4. In some embodiments, N is 5, 6, 7, 8, 9, 10, 11, 12, 13, 14, 15, 20, 50, 100, 1000, 10,000, 100,000, 500,000, 1×10⁶, 5×10⁶, 1×10⁷, or greater.

In some embodiments, the model comprises a plurality of at least 500 parameters. In some embodiments, the plurality of parameters comprises at least 10, at least 50, at least 100, at least 500, at least 1000, at least 2000, at least 5000, at least 10,000, at least 20,000, at least 50,000, at least 100,000, at least 200,000, at least 500,000, at least 1 million, at least 2 million, at least 3 million, at least 4 million or at least 5 million parameters. In some embodiments, the plurality of parameters comprises no more than 8 million, no more than 5 million, no more than 4 million, no more than 1 million, no more than 500,000, no more than 100,000, no more than 50,000, no more than 10,000, no more than 5000, no more than 1000, or no more than 500 parameters. In some embodiments, the plurality of parameters comprises from 10 to 5000, from 500 to 10,000, from 10,000 to 500,000, from 20,000 to 1 million, or from 1 million to 5 million parameters. In some embodiments, the plurality of parameters falls within another range starting no lower than 10 parameters and ending no higher than 8 million parameters.

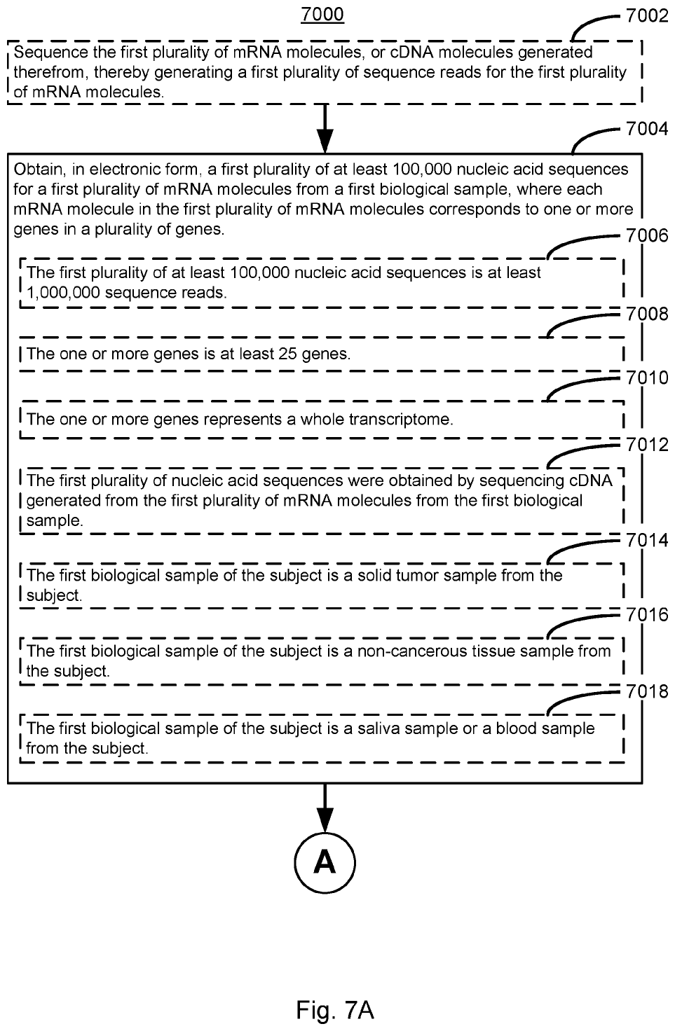

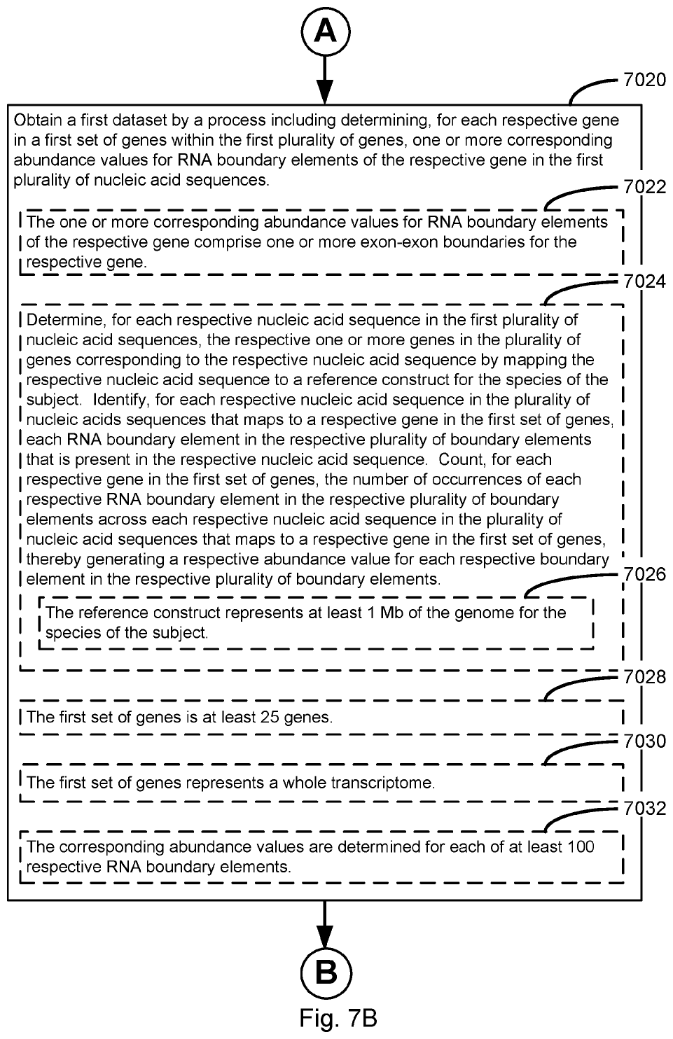

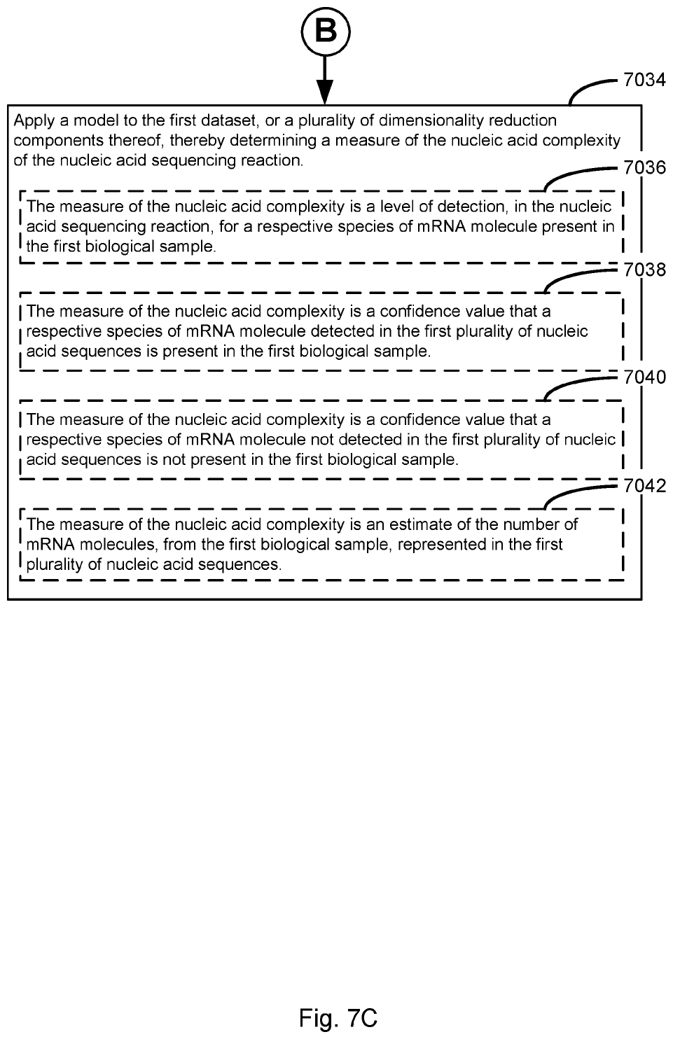

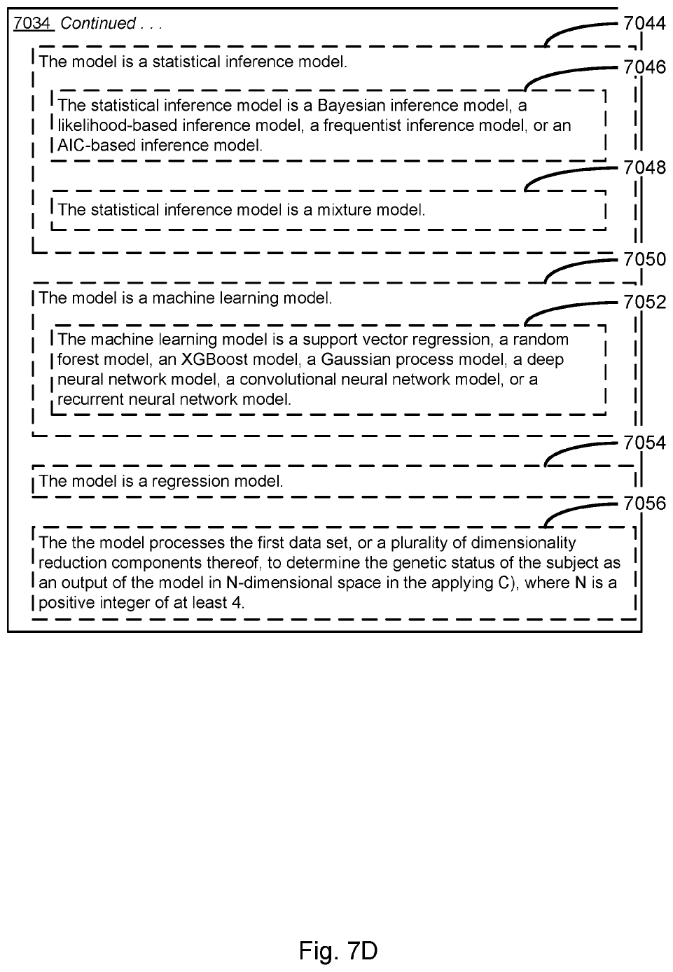

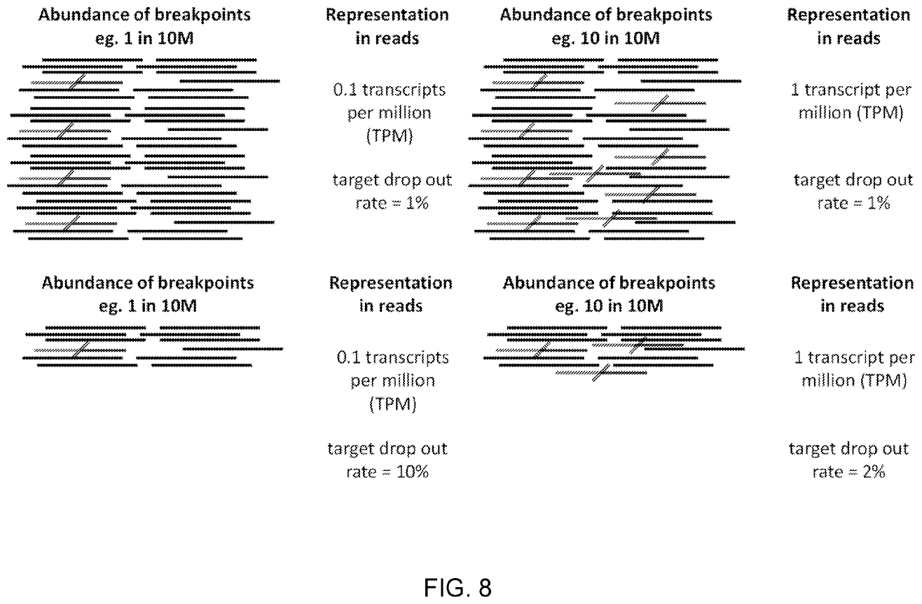

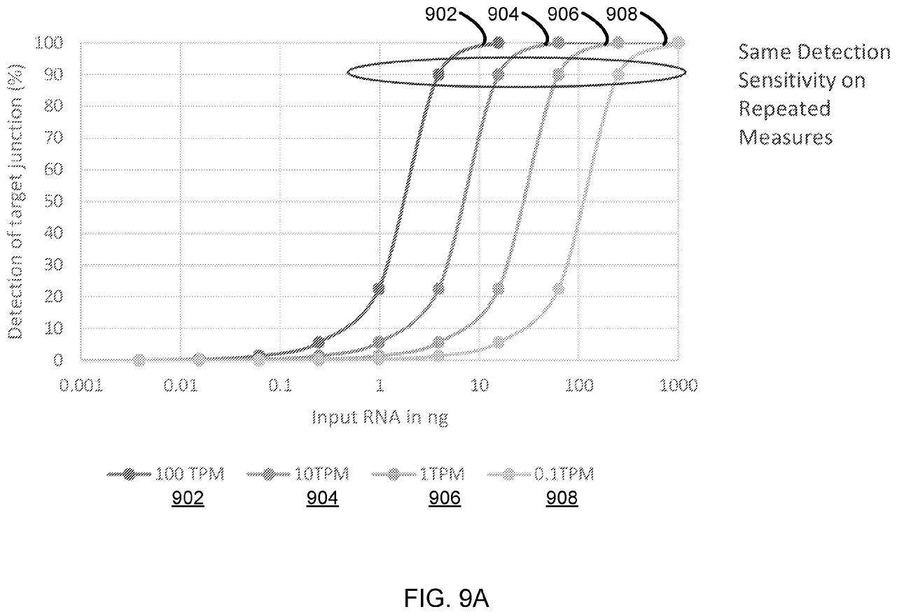

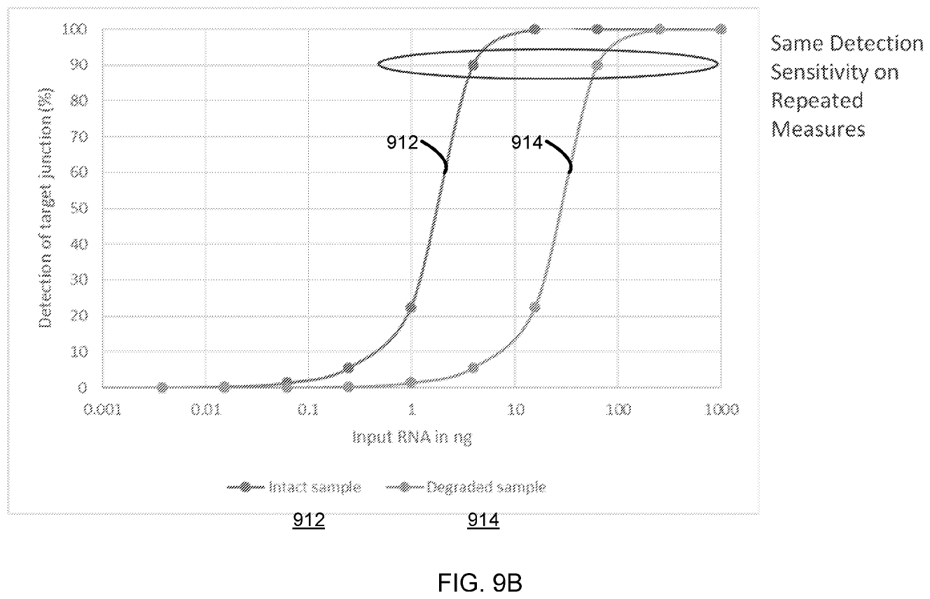

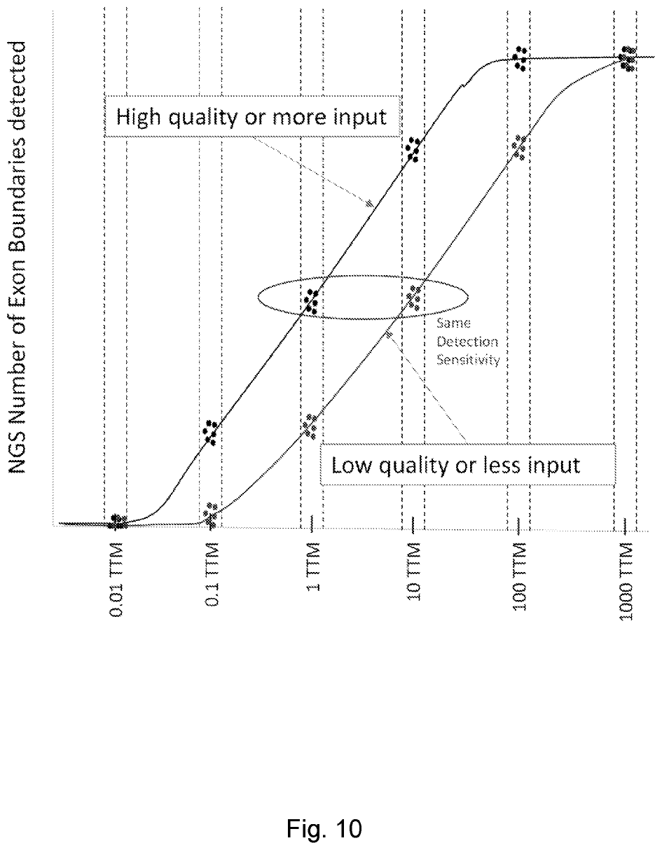

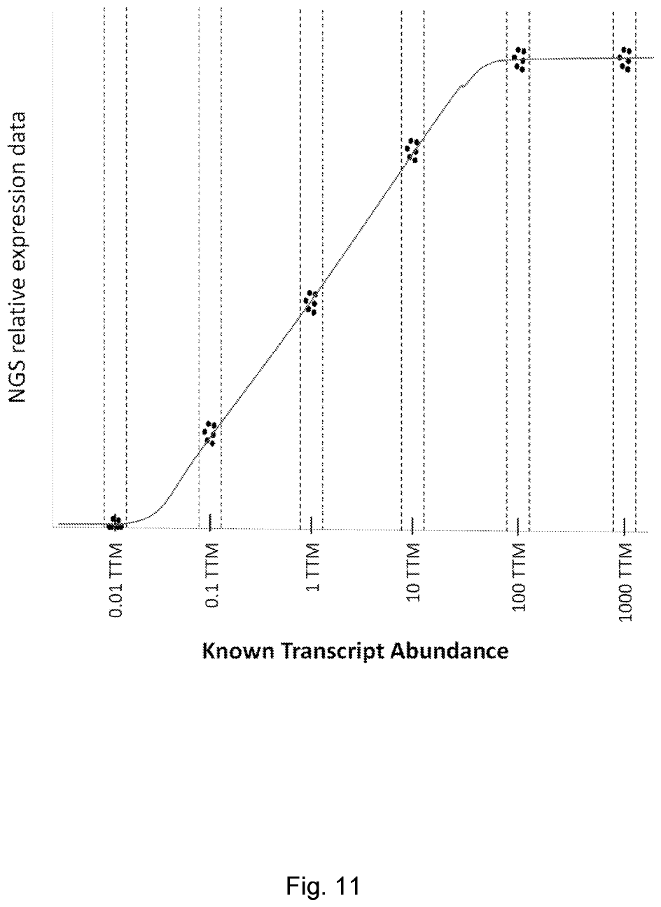

In some embodiments, the method includes determining a confidence score for the genetic status of the subject (5102), e.g., using any of the methods described herein with reference to FIGS. 4C and 7A-7D. In some embodiments, the confidence value is dependent upon a measure of sequencing depth for the first plurality of nucleic acid sequences (5104). In some embodiments, the confidence value is dependent upon the presence or absence of orthogonal evidence for the genetic status (5106), e.g., direct or indirect evidence for the fusion in a genomic sequencing reaction for a biological sample of the subject.

Example Methods for Evaluating mRNA Isoforms

In some embodiments, the disclosure provides a method of determining a status for an mRNA transcript splice variant in a first tissue of a test subject. The method is performed at a computer system having one or more processors, and memory storing one or more programs for execution by the one or more processors. The method includes obtaining, from a first sequencing reaction, a first plurality of sequences, in electronic form, of a first plurality of mRNA molecules in a first biological sample of the first tissue of the test subject, wherein each mRNA molecule in the first plurality of mRNA molecules corresponds to one or more genes in a plurality of genes. The method then includes determining, for each respective gene in a first set of genes within the plurality of genes, a first corresponding RNA boundary distribution comprising a corresponding relative abundance value (e.g., where the abundance value is a value for the number of unique occurrences of the RNA boundary sub-sequence) for each respective RNA boundary sub-sequence in a plurality of RNA boundary sub-sequences of the respective gene (e.g., each possible exon-exon boundary of the gene) using the first plurality of sequences. In some embodiments, abundances for one or more gene fusion boundaries are also determined. The method then includes performing a procedure for each respective gene in the first set of genes, the procedure including evaluating the first corresponding RNA boundary distribution for the respective gene with a model that has been trained to detect mRNA transcript splice variants based on RNA boundary distributions, and determining, responsive to the evaluating, a respective indication (e.g., a probability, likelihood, dichotomous or binary prediction, prevalence, or isoform distribution) whether an mRNA transcript splice variant of the respective gene is present in the first tissue.

In some embodiments, the test subject has cancer. Non-limiting examples of cancer types include ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, breast cancer, non-clear cell renal cell carcinoma, glioblastoma, glioma, kidney cancer, gastrointestinal stromal tumor, medulloblastoma, bladder cancer, gastric cancer, bone cancer, non-small cell lung cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, Her2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and papillary renal cell carcinoma.

In some embodiments, the first sequencing reaction is whole exome RNA sequencing. In some embodiments, the first sequencing reaction is targeted-panel RNA sequencing.

RNA-seq is a methodology for RNA profiling based on next-generation sequencing that enables the measurement and comparison of gene expression patterns across a plurality of subjects. In some embodiments, millions of short strings, called ‘sequence reads,’ are generated from sequencing random positions of cDNA prepared from the input RNAs that are obtained from tumor tissue of a subject. These reads can then be computationally mapped on a reference genome to reveal a ‘transcriptional map’, where the number of sequence reads aligned to each gene gives a measure of its level of expression (e.g., abundance). Next-generation sequencing is disclosed in Shendure, 2008, “Next-generation DNA sequencing,” Nat. Biotechnology 26, pp. 1135-1145, which is hereby incorporated by reference. RNA-seq is disclosed in Nagalakshmi et al., 2008, “The transcriptional landscape of the yeast genome defined by RNA sequencing,” Science 320, pp. 1344-1349; and Finotell and Camillo, 2014, “Measuring differential gene expression with RNA-seq: challenges and strategies for data analysis,” Briefings in Functional Genomics 14(2), pp. 130-142, each of which is hereby incorporated by reference.

Generally, RNA sequencing begins by fragmenting RNAs in the sample of interest and reverse-transcribing the fragments into complementary DNAs (cDNAs). The obtained cDNAs are then amplified and subjected to next-generation DNA sequencing (NGS). In principle, any NGS technology can be used for RNA-seq. In some embodiments, the Illumina sequencer (see the Internet at illumina.com) is used. See, Wang, Z., et al., “RNA-Seq: a revolutionary tool for transcriptomics,” Nat Rev Genet., 10(1):57-63 (2009), which is hereby incorporated by reference.

Conventionally, the millions of short reads generated for each such sample are then mapped on a reference construct (e.g., a reference genome), by identifying gene regions that match read sequences. Any of a variety of alignment tools can be used for this task. See, for example, Hatem et al., 2013, “Benchmarking short sequence mapping tools,” BMC Bioinformatics 14, p. 184; and Engstrom et al., “Systematic evaluation of spliced alignment programs for RNA-seq data, Nat Methods 10, pp. 1185-1191, each of which is hereby incorporated by reference. In some embodiments, the mapping process starts by building an index of either the reference genome or the reads, which is then used to retrieve the set of positions in the reference sequence where the reads are more likely to align. Once this subset of possible mapping locations has been identified, alignment is performed in these candidate regions with slower and more sensitive algorithms. See, for example, Hatem et al., 2013, “Benchmarking short sequence mapping tools,” BMC Bioinformatics 14: p. 184; and Flicek and Birney, 2009, “Sense from sequence reads: methods for alignment and assembly,” Nat Methods 6(Suppl. 11), S6-S12, each of which is hereby incorporated by reference. In some embodiments, the mapping tools is a methodology that makes use of a hash table or makes use of a Burrows-Wheeler transform (BWT). See, for example, Li and Homer, 2010, “A survey of sequence alignment algorithms for next-generation sequencing,” Brief Bioinformatics 11, pp. 473-483, which is hereby incorporated by reference. After mapping, the reads aligned to each coding unit, such as exon, transcript or gene, are used to compute counts, in order to provide an estimate of its abundance (e.g., expression) level.

Quantification of gene abundance from RNA-seq data is conventionally implemented through two computational steps: alignment of reads to a reference genome or transcriptome, and subsequent estimation of gene and isoform abundances based on aligned reads. Unfortunately, the reads generated by the most used RNA-Seq technologies are generally much shorter than the transcripts from which they are sampled. As a consequence, in the presence of transcripts with similar sequences, it is not always possible to uniquely assign short sequence reads to a specific gene. Such sequence reads are referred to as “multireads” because they are homologous to more than one region of the reference genome. In some embodiments, such multireads are discarded, that is, they do not contribute to gene abundance counts. In some embodiments, programs such as MMSEQ or RSEM, are used to resolve the ambiguity. See for example, Turro et al., 2011, “Haplotype and isoform specific expression estimation using multi-mapping RNAseq reads,” Genome Biol 12, p. R13; and Nicolae et al., “Estimation of alternative splicing isoform frequencies from RNA-Seq data,” Algorithms Mol Biol 6, p. 9, each of which is hereby incorporated by reference. Advantageously, in some embodiments, the methods and systems described herein overcome the ambiguity associated with multireads by counting exon boundaries (e.g., exon/exon junctions, rather than relying on alignment of the entire sequence of the reads.

In some embodiments, the plurality of sequences, generated from the RNA sequencing reaction, is at least 10,000 sequences. In other embodiments, the plurality of sequences generated from the RNA sequencing reaction, is at least 50,000 sequences, 100,000 sequences, 250,000 sequences, 500,000 sequences, 1 million sequences, 2.5 million sequences, 5 million sequences, 10 million sequences, or more. In some embodiments, the plurality of sequences, generated from the RNA sequencing reaction, is from 10,000 sequences to 10 million sequences. In some embodiments, the plurality of sequences, generated from the RNA sequencing reaction, is from 100,000 sequences to 10 million sequences. In some embodiments, the plurality of sequences, generated from the RNA sequencing reaction, is from 1 million sequences to 10 million sequences. In some embodiments, the plurality of sequences, generated from the RNA sequencing reaction, is from 10,000 sequences to 5 million sequences. In some embodiments, the plurality of sequences, generated from the RNA sequencing reaction, is from 100,000 sequences to 5 million sequences. In some embodiments, the plurality of sequences, generated from the RNA sequencing reaction, is from 1 million sequences to 5 million sequences. In some embodiments, the plurality of sequences, generated from the RNA sequencing reaction, is from 10,000 sequences to 2.5 million sequences. In some embodiments, the plurality of sequences, generated from the RNA sequencing reaction, is from 100,000 sequences to 2.5 million sequences. In some embodiments, the plurality of sequences, generated from the RNA sequencing reaction, is from 1 million sequences to 2.5 million sequences. In some embodiments, the plurality of sequences, generated from the RNA sequencing reaction, is from 10,000 sequences to 1 million sequences. In some embodiments, the plurality of sequences, generated from the RNA sequencing reaction, is from 100,000 sequences to 1 million sequences.

In some embodiments, the biological sample of the test subject is a cancerous tissue. In some embodiments, the cancerous tissue biopsy is a solid tumor biopsy. Methods for obtaining solid tissue samples, e.g., of cancerous and/or normal tissue are known in the art and are dependent upon the type of tissue being sampled. For example, bone marrow biopsies and isolation of circulating tumor cells can be used to obtain samples of blood cancers, endoscopic biopsies can be used to obtain samples of cancers of the digestive tract, bladder, and lungs, needle biopsies (e.g., fine-needle aspiration, core needle aspiration, vacuum-assisted biopsy, and image-guided biopsy, can be used to obtain samples of subdermal tumors, skin biopsies, e.g., shave biopsy, punch biopsy, incisional biopsy, and excisional biopsy, can be used to obtain samples of dermal cancers, and surgical biopsies can be used to obtain samples of cancers affecting internal organs of a patient. In some embodiments, a solid tissue sample is a formalin-fixed tissue (FFT). In some embodiments, a solid tissue sample is a macro-dissected formalin fixed paraffin embedded (FFPE) tissue. In some embodiments, a solid tissue sample is a fresh frozen tissue sample.

In some embodiments, a dedicated normal sample is collected from the patient, for co-processing with a liquid biopsy sample. Generally, the normal sample is of a non-cancerous tissue, and can be collected using any tissue collection means described above. In some embodiments, buccal cells collected from the inside of a patient's cheeks are used as a normal sample. Buccal cells can be collected by placing an absorbent material, e.g., a swab, in the subject's mouth and rubbing it against their cheek, e.g., for at least 15 second or for at least 30 seconds. The swab is then removed from the patient's mouth and inserted into a tube, such that the tip of the tube is submerged into a liquid that serves to extract the buccal cells off of the absorbent material. An example of buccal cell recovery and collection devices is provided in U.S. Pat. No. 9,138,205, the content of which is hereby incorporated by reference, in its entirety, for all purposes. In some embodiments, the buccal swab DNA is used as a source of normal DNA in circulating heme malignancies.

In some embodiments, the plurality of genes is at least 100 genes. In other embodiments, the plurality of genes is at least 10 genes, at least 25 genes, at least 50 genes, at least 75 genes, at least 125 genes, at least 150 genes, at least 200 genes, at least 250 genes, at least 300 genes, at least 400 genes, at least 500 genes, at least 750 genes, at least 1000 genes, at least 2500 genes, at least 5000 genes, at least 7500 genes, at least 10,000 genes, at least 20,000 genes, or more. In some embodiments, the plurality of genes is from 10 genes to 20,000 genes. In some embodiments, the plurality of genes is from 25 genes to 20,000 genes. In some embodiments, the plurality of genes is from 50 genes to 20,000 genes. In some embodiments, the plurality of genes is from 100 genes to 20,000 genes. In some embodiments, the plurality of genes is from 250 genes to 20,000 genes. In some embodiments, the plurality of genes is from 500 genes to 20,000 genes. In some embodiments, the plurality of genes is from 1000 genes to 20,000 genes. In some embodiments, the plurality of genes is from 2500 genes to 20,000 genes. In some embodiments, the plurality of genes is from 5000 genes to 20,000 genes. In some embodiments, the plurality of genes is from 10,000 genes to 20,000 genes. In some embodiments, the plurality of genes is from 10 genes to 10,000 genes. In some embodiments, the plurality of genes is from 25 genes to 10,000 genes. In some embodiments, the plurality of genes is from 50 genes to 10,000 genes. In some embodiments, the plurality of genes is from 100 genes to 10,000 genes. In some embodiments, the plurality of genes is from 250 genes to 10,000 genes. In some embodiments, the plurality of genes is from 500 genes to 10,000 genes. In some embodiments, the plurality of genes is from 1000 genes to 10,000 genes. In some embodiments, the plurality of genes is from 2500 genes to 10,000 genes. In some embodiments, the plurality of genes is from 5000 genes to 10,000 genes. In some embodiments, the plurality of genes is from 10 genes to 5000 genes. In some embodiments, the plurality of genes is from 25 genes to 5000 genes. In some embodiments, the plurality of genes is from 50 genes to 5000 genes. In some embodiments, the plurality of genes is from 100 genes to 5000 genes. In some embodiments, the plurality of genes is from 250 genes to 5000 genes. In some embodiments, the plurality of genes is from 500 genes to 5000 genes. In some embodiments, the plurality of genes is from 1000 genes to 5000 genes. In some embodiments, the plurality of genes is from 2500 genes to 5000 genes. In some embodiments, the plurality of genes is from 10 genes to 1000 genes. In some embodiments, the plurality of genes is from 25 genes to 1000 genes. In some embodiments, the plurality of genes is from 50 genes to 1000 genes. In some embodiments, the plurality of genes is from 100 genes to 1000 genes. In some embodiments, the plurality of genes is from 250 genes to 1000 genes. In some embodiments, the plurality of genes is from 500 genes to 1000 genes. In some embodiments, the plurality of genes is from 10 genes to 500 genes. In some embodiments, the plurality of genes is from 25 genes to 500 genes. In some embodiments, the plurality of genes is from 50 genes to 500 genes. In some embodiments, the plurality of genes is from 100 genes to 500 genes. In some embodiments, the plurality of genes is from 250 genes to 500 genes. In some embodiments, the plurality of genes is from 10 genes to 250 genes. In some embodiments, the plurality of genes is from 25 genes to 250 genes. In some embodiments, the plurality of genes is from 50 genes to 250 genes. In some embodiments, the plurality of genes is from 100 genes to 250 genes. In some embodiments, the plurality of genes is from 10 genes to 100 genes. In some embodiments, the plurality of genes is from 25 genes to 100 genes. In some embodiments, the plurality of genes is from 50 genes to 100 genes.

In some embodiments, the test subject has a neuropsychiatric disorder. Accordingly, in some embodiments, the biological sample of the test subject is a non-cancerous tissue sample.

In some embodiments, for a respective gene in the plurality of genes, the plurality of RNA boundary sub-sequences for the respective gene includes at least one RNA boundary sub-sequence for a junction between non-consecutive exons in the respective gene. For instance, for a gene having 4 exons, exon boundaries between exons 1/2, 2/3, and 3/4 represent consecutive exon boundaries, while one or more boundaries between exons 1/3, 1/4, and 2/4 may also be identified by the methods and systems described herein. For example, in some embodiments, for a respective gene in the plurality of genes, the plurality of RNA boundary sub-sequences for the respective gene includes RNA boundary sub-sequences for each junction between non-consecutive exons in one or more known mRNA splice isoforms for the respective gene.

In some embodiments, for a first respective gene in the plurality of genes, the plurality of RNA boundary sub-sequences for the respective gene includes at least one RNA boundary subsequence for a junction between the respective gene and another gene.

In some embodiments, for a first respective gene in the plurality of genes, the plurality of RNA boundary sub-sequences for the respective gene includes at least one RNA boundary subsequence for a junction formed between an aberrant splice site in a first exon and a canonical splice site in a second exon.

In some embodiments, each instance of the evaluating includes applying the model to the first corresponding RNA boundary distributions for the respective gene.

In some embodiments, each instance of the evaluating includes determining a first corresponding exon distribution for the respective gene from the first corresponding RNA boundary distribution for the respective gene, the first corresponding exon distribution comprising a corresponding relative abundance value for each respective exon in a first plurality of exons of the first gene, and applying the model to the first corresponding exon distribution for the respective gene.

In some embodiments, the model is trained such that the determining may indicate that a respective mRNA transcript splice variant of the first gene is present in the first tissue when the first plurality of sequences does not include a sequence spanning any junction between non-consecutive exons in the respective mRNA transcript splice variant.

In some embodiments, the determining also includes determining, for each respective gene in the first set of genes within the plurality of genes, a second corresponding RNA boundary distribution comprising a corresponding relative abundance value for each respective RNA boundary sub-sequence in the plurality of RNA boundary sub-sequences of the respective gene in a second plurality of sequences, obtained in a second sequencing reaction, for a second plurality of mRNA molecules in a second biological sample of a second tissue of the test subject, and the evaluating also includes evaluating the second corresponding RNA boundary distributions for the respective gene from the determining with the model. In some embodiments, the second tissue of the subject is not the same type of tissue as the first tissue. In some embodiments, the first tissue of the subject is a cancerous tissue of the subject and the second tissue of the subject is a non-cancerous tissue of the subject.

In some embodiments, the model is trained to provide a probability, likelihood, dichotomous prediction, or binary prediction for whether a respective mRNA transcript splice variant for the respective gene is present in the tissue of the test subject.

In some embodiments, the model is trained to provide a relative abundance for a respective mRNA transcript splice variant for the respective gene in the tissue of the test subject.

In some embodiments, the model is trained to provide, for each respective mRNA transcript splice variant in a plurality of mRNA transcript splice variants for the respective gene, a respective relative abundance of the respective mRNA transcript splice variant in the tissue of the test subject.

In some embodiments, the model is trained specifically for determining a status for an mRNA transcript splice variant of the respective gene.

In some embodiments, the determining also includes evaluating a tissue type of the first tissue of the test subject using the model.

In some embodiments, the model is trained specifically for determining a status for an mRNA transcript splice variant in a tissue type of the first tissue of the test subject.

In some embodiments, the method also includes generating, based on the first plurality of sequences, a sample-specific distribution (e.g., a standard curve) of measures of relative abundance {e.g., a number of unique reads containing an RNA boundary sub-sequence for the gene} for mRNA molecules corresponding to each respective gene, in a second set of genes within the plurality of genes, in the first biological sample, and determining a respective measure of confidence for the indication by comparing (a) a measure of relative abundance for the mRNA transcript splice variant in the first biological sample, to (b) the sample-specific distribution.

In some embodiments, the comparing the measure of relative abundance for the mRNA transcript splice variant to the sample-specific distribution includes determining a level of detection, at a first level of confidence, for an mRNA transcript in the first plurality of sequences, and comparing a level of support for the mRNA transcript splice variant in the first plurality of sequences to the level of detection.

Example Methods for Detecting Gene Fusions

In some embodiments, the disclosure provides a method of determining a status for a gene fusion in a first tissue of a test subject. The methods is performed at a computer system having one or more processors, and memory storing one or more programs for execution by the one or more processors. The method includes obtaining, from a first sequencing reaction, a first plurality of sequences, in electronic form, of a first plurality of mRNA molecules in a first biological sample of the first tissue of the test subject, wherein each mRNA molecule in the first plurality of mRNA molecules corresponds to one or more genes in a plurality of genes. The method then includes determining, for each respective gene in a first set of genes within the first plurality of genes, a first corresponding RNA boundary distribution comprising a corresponding relative abundance value (e.g., where the abundance value is a value for the number of unique occurrences of the RNA boundary sub-sequence in the plurality of sequences) for each respective RNA boundary sub-sequence in a plurality of RNA boundary sub-sequences of the respective gene (e.g., each possible exon-exon boundary of the gene) using the first plurality of sequences. In some embodiments, abundances for one or more gene fusion boundaries are also determined. The method then includes performing a procedure for each respective pair of genes in one or more pairs of genes present in the first set of genes. The procedure includes evaluating the first corresponding RNA boundary distribution for a first gene in the respective pair of genes and the first corresponding RNA boundary distribution for a second gene in the respective pair of genes, as determined, with a model that has been trained to detect gene fusions based on RNA boundary distributions. The procedure also includes determining, responsive to the evaluating (i), a respective indication (e.g., a probability, likelihood, dichotomous or binary prediction) whether a gene fusion between the respective pair of genes is present in the first tissue from an output of the model.

In some embodiments, the test subject has cancer. Non-limiting examples of cancer types include ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, breast cancer, non-clear cell renal cell carcinoma, glioblastoma, glioma, kidney cancer, gastrointestinal stromal tumor, medulloblastoma, bladder cancer, gastric cancer, bone cancer, non-small cell lung cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, Her2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and papillary renal cell carcinoma.

In some embodiments, the first sequencing reaction is whole exome RNA sequencing. In some embodiments, the first sequencing reaction is targeted-panel RNA sequencing.

RNA-seq is a methodology for RNA profiling based on next-generation sequencing that enables the measurement and comparison of gene expression patterns across a plurality of subjects. In some embodiments, millions of short strings, called ‘sequence reads,’ are generated from sequencing random positions of cDNA prepared from the input RNAs that are obtained from tumor tissue of a subject. These reads can then be computationally mapped on a reference genome to reveal a ‘transcriptional map’, where the number of sequence reads aligned to each gene gives a measure of its level of expression (e.g., abundance). Next-generation sequencing is disclosed in Shendure, 2008, “Next-generation DNA sequencing,” Nat. Biotechnology 26, pp. 1135-1145, which is hereby incorporated by reference. RNA-seq is disclosed in Nagalakshmi et al., 2008, “The transcriptional landscape of the yeast genome defined by RNA sequencing,” Science 320, pp. 1344-1349; and Finotell and Camillo, 2014, “Measuring differential gene expression with RNA-seq: challenges and strategies for data analysis,” Briefings in Functional Genomics 14(2), pp. 130-142, each of which is hereby incorporated by reference.

Generally, RNA sequencing begins by fragmenting RNAs in the sample of interest and reverse-transcribing the fragments into complementary DNAs (cDNAs). The obtained cDNAs are then amplified and subjected to next-generation DNA sequencing (NGS). In principle, any NGS technology can be used for RNA-seq. In some embodiments, the Illumina sequencer (see the Internet at illumina.com) is used. See, Wang, Z., et al., “RNA-Seq: a revolutionary tool for transcriptomics,” Nat Rev Genet., 10(1):57-63 (2009), which is hereby incorporated by reference.

Conventionally, the millions of short reads generated for each such sample are then mapped on a reference construct (e.g., a reference genome), by identifying gene regions that match read sequences. Any of a variety of alignment tools can be used for this task. See, for example, Hatem et al., 2013, “Benchmarking short sequence mapping tools,” BMC Bioinformatics 14, p. 184; and Engstrom et al., “Systematic evaluation of spliced alignment programs for RNA-seq data, Nat Methods 10, pp. 1185-1191, each of which is hereby incorporated by reference. In some embodiments, the mapping process starts by building an index of either the reference genome or the reads, which is then used to retrieve the set of positions in the reference sequence where the reads are more likely to align. Once this subset of possible mapping locations has been identified, alignment is performed in these candidate regions with slower and more sensitive algorithms. See, for example, Hatem et al., 2013, “Benchmarking short sequence mapping tools,” BMC Bioinformatics 14: p. 184; and Flicek and Birney, 2009, “Sense from sequence reads: methods for alignment and assembly,” Nat Methods 6(Suppl. 11), S6-S12, each of which is hereby incorporated by reference. In some embodiments, the mapping tools is a methodology that makes use of a hash table or makes use of a Burrows-Wheeler transform (BWT). See, for example, Li and Homer, 2010, “A survey of sequence alignment algorithms for next-generation sequencing,” Brief Bioinformatics 11, pp. 473-483, which is hereby incorporated by reference. After mapping, the reads aligned to each coding unit, such as exon, transcript or gene, are used to compute counts, in order to provide an estimate of its abundance (e.g., expression) level.

Quantification of gene abundance from RNA-seq data is conventionally implemented through two computational steps: alignment of reads to a reference genome or transcriptome, and subsequent estimation of gene and isoform abundances based on aligned reads. Unfortunately, the reads generated by the most used RNA-Seq technologies are generally much shorter than the transcripts from which they are sampled. As a consequence, in the presence of transcripts with similar sequences, it is not always possible to uniquely assign short sequence reads to a specific gene. Such sequence reads are referred to as “multireads” because they are homologous to more than one region of the reference genome. In some embodiments, such multireads are discarded, that is, they do not contribute to gene abundance counts. In some embodiments, programs such as MMSEQ or RSEM, are used to resolve the ambiguity. See for example, Turro et al., 2011, “Haplotype and isoform specific expression estimation using multi-mapping RNAseq reads,” Genome Biol 12, p. R13; and Nicolae et al., “Estimation of alternative splicing isoform frequencies from RNA-Seq data,” Algorithms Mol Biol 6, p. 9, each of which is hereby incorporated by reference. Advantageously, in some embodiments, the methods and systems described herein overcome the ambiguity associated with multireads by counting exon boundaries (e.g., exon/exon junctions, rather than relying on alignment of the entire sequence of the reads.

In some embodiments, the plurality of sequences, generated from the RNA sequencing reaction, is at least 10,000 sequences. In other embodiments, the plurality of sequences generated from the RNA sequencing reaction, is at least 50,000 sequences, 100,000 sequences, 250,000 sequences, 500,000 sequences, 1 million sequences, 2.5 million sequences, 5 million sequences, 10 million sequences, or more. In some embodiments, the plurality of sequences, generated from the RNA sequencing reaction, is from 10,000 sequences to 10 million sequences. In some embodiments, the plurality of sequences, generated from the RNA sequencing reaction, is from 100,000 sequences to 10 million sequences. In some embodiments, the plurality of sequences, generated from the RNA sequencing reaction, is from 1 million sequences to 10 million sequences. In some embodiments, the plurality of sequences, generated from the RNA sequencing reaction, is from 10,000 sequences to 5 million sequences. In some embodiments, the plurality of sequences, generated from the RNA sequencing reaction, is from 100,000 sequences to 5 million sequences. In some embodiments, the plurality of sequences, generated from the RNA sequencing reaction, is from 1 million sequences to 5 million sequences. In some embodiments, the plurality of sequences, generated from the RNA sequencing reaction, is from 10,000 sequences to 2.5 million sequences. In some embodiments, the plurality of sequences, generated from the RNA sequencing reaction, is from 100,000 sequences to 2.5 million sequences. In some embodiments, the plurality of sequences, generated from the RNA sequencing reaction, is from 1 million sequences to 2.5 million sequences. In some embodiments, the plurality of sequences, generated from the RNA sequencing reaction, is from 10,000 sequences to 1 million sequences. In some embodiments, the plurality of sequences, generated from the RNA sequencing reaction, is from 100,000 sequences to 1 million sequences.

In some embodiments, the biological sample of the test subject is a cancerous tissue. In some embodiments, the cancerous tissue biopsy is a solid tumor biopsy. Methods for obtaining solid tissue samples, e.g., of cancerous and/or normal tissue are known in the art and are dependent upon the type of tissue being sampled. For example, bone marrow biopsies and isolation of circulating tumor cells can be used to obtain samples of blood cancers, endoscopic biopsies can be used to obtain samples of cancers of the digestive tract, bladder, and lungs, needle biopsies (e.g., fine-needle aspiration, core needle aspiration, vacuum-assisted biopsy, and image-guided biopsy, can be used to obtain samples of subdermal tumors, skin biopsies, e.g., shave biopsy, punch biopsy, incisional biopsy, and excisional biopsy, can be used to obtain samples of dermal cancers, and surgical biopsies can be used to obtain samples of cancers affecting internal organs of a patient. In some embodiments, a solid tissue sample is a formalin-fixed tissue (FFT). In some embodiments, a solid tissue sample is a macro-dissected formalin fixed paraffin embedded (FFPE) tissue. In some embodiments, a solid tissue sample is a fresh frozen tissue sample.

In some embodiments, a dedicated normal sample is collected from the patient, for co-processing with a liquid biopsy sample. Generally, the normal sample is of a non-cancerous tissue, and can be collected using any tissue collection means described above. In some embodiments, buccal cells collected from the inside of a patient's cheeks are used as a normal sample. Buccal cells can be collected by placing an absorbent material, e.g., a swab, in the subject's mouth and rubbing it against their cheek, e.g., for at least 15 second or for at least 30 seconds. The swab is then removed from the patient's mouth and inserted into a tube, such that the tip of the tube is submerged into a liquid that serves to extract the buccal cells off of the absorbent material. An example of buccal cell recovery and collection devices is provided in U.S. Pat. No. 9,138,205, the content of which is hereby incorporated by reference, in its entirety, for all purposes. In some embodiments, the buccal swab DNA is used as a source of normal DNA in circulating heme malignancies.

In some embodiments, the plurality of genes is at least 100 genes. In other embodiments, the plurality of genes is at least 10 genes, at least 25 genes, at least 50 genes, at least 75 genes, at least 125 genes, at least 150 genes, at least 200 genes, at least 250 genes, at least 300 genes, at least 400 genes, at least 500 genes, at least 750 genes, at least 1000 genes, at least 2500 genes, at least 5000 genes, at least 7500 genes, at least 10,000 genes, at least 20,000 genes, or more. In some embodiments, the plurality of genes is from 10 genes to 20,000 genes. In some embodiments, the plurality of genes is from 25 genes to 20,000 genes. In some embodiments, the plurality of genes is from 50 genes to 20,000 genes. In some embodiments, the plurality of genes is from 100 genes to 20,000 genes. In some embodiments, the plurality of genes is from 250 genes to 20,000 genes. In some embodiments, the plurality of genes is from 500 genes to 20,000 genes. In some embodiments, the plurality of genes is from 1000 genes to 20,000 genes. In some embodiments, the plurality of genes is from 2500 genes to 20,000 genes. In some embodiments, the plurality of genes is from 5000 genes to 20,000 genes. In some embodiments, the plurality of genes is from 10,000 genes to 20,000 genes. In some embodiments, the plurality of genes is from 10 genes to 10,000 genes. In some embodiments, the plurality of genes is from 25 genes to 10,000 genes. In some embodiments, the plurality of genes is from 50 genes to 10,000 genes. In some embodiments, the plurality of genes is from 100 genes to 10,000 genes. In some embodiments, the plurality of genes is from 250 genes to 10,000 genes. In some embodiments, the plurality of genes is from 500 genes to 10,000 genes. In some embodiments, the plurality of genes is from 1000 genes to 10,000 genes. In some embodiments, the plurality of genes is from 2500 genes to 10,000 genes. In some embodiments, the plurality of genes is from 5000 genes to 10,000 genes. In some embodiments, the plurality of genes is from 10 genes to 5000 genes. In some embodiments, the plurality of genes is from 25 genes to 5000 genes. In some embodiments, the plurality of genes is from 50 genes to 5000 genes. In some embodiments, the plurality of genes is from 100 genes to 5000 genes. In some embodiments, the plurality of genes is from 250 genes to 5000 genes. In some embodiments, the plurality of genes is from 500 genes to 5000 genes. In some embodiments, the plurality of genes is from 1000 genes to 5000 genes. In some embodiments, the plurality of genes is from 2500 genes to 5000 genes. In some embodiments, the plurality of genes is from 10 genes to 1000 genes. In some embodiments, the plurality of genes is from 25 genes to 1000 genes. In some embodiments, the plurality of genes is from 50 genes to 1000 genes. In some embodiments, the plurality of genes is from 100 genes to 1000 genes. In some embodiments, the plurality of genes is from 250 genes to 1000 genes. In some embodiments, the plurality of genes is from 500 genes to 1000 genes. In some embodiments, the plurality of genes is from 10 genes to 500 genes. In some embodiments, the plurality of genes is from 25 genes to 500 genes. In some embodiments, the plurality of genes is from 50 genes to 500 genes. In some embodiments, the plurality of genes is from 100 genes to 500 genes. In some embodiments, the plurality of genes is from 250 genes to 500 genes. In some embodiments, the plurality of genes is from 10 genes to 250 genes. In some embodiments, the plurality of genes is from 25 genes to 250 genes. In some embodiments, the plurality of genes is from 50 genes to 250 genes. In some embodiments, the plurality of genes is from 100 genes to 250 genes. In some embodiments, the plurality of genes is from 10 genes to 100 genes. In some embodiments, the plurality of genes is from 25 genes to 100 genes. In some embodiments, the plurality of genes is from 50 genes to 100 genes.

In some embodiments, for a respective gene in the plurality of genes, the plurality of RNA boundary sub-sequences for the respective gene includes at least one RNA boundary sub-sequence for a junction between non-consecutive exons in the respective gene. For instance, for a gene having 4 exons, exon boundaries between exons 1/2, 2/3, and 3/4 represent consecutive exon boundaries, while one or more boundaries between exons 1/3, 1/4, and 2/4 may also be identified by the methods and systems described herein. For example, in some embodiments, for a respective gene in the plurality of genes, the plurality of RNA boundary sub-sequences for the respective gene includes RNA boundary sub-sequences for each junction between non-consecutive exons in one or more known mRNA splice isoforms for the respective gene.

Similarly, in some embodiments, for a first respective gene in the plurality of genes, the plurality of RNA boundary sub-sequences for the respective gene includes at least one RNA boundary subsequence for a junction between the respective first gene and a second respective gene in the plurality of genes.

In some embodiments, each instance of the evaluating includes applying the model to the first corresponding RNA boundary distribution for the first gene in the respective pair of genes and the first corresponding RNA boundary distribution for the second gene in the respective pair of genes.

In some embodiments, each instance of the evaluating includes determining a first corresponding exon distribution for the first gene in the respective pair of genes from the first corresponding RNA boundary distribution for the first gene in the respective pair of genes, the first corresponding exon distribution for the first gene comprising a corresponding relative abundance value for each respective exon in a plurality of exons of the first gene; determining a first corresponding exon distribution for the second gene in the respective pair of genes from the first corresponding RNA boundary distribution for the second gene in the respective pair of genes, the first corresponding exon distribution for the second gene comprising a corresponding relative abundance value for each respective exon in a plurality of exons of the second gene; and applying the model to the first corresponding exon distribution for the first gene in the respective pair of genes and the first corresponding exon distribution for the second gene in the respective pair of genes.

In some embodiments, the model is trained such that the determining may indicate that a respective gene fusion between the respective pair of genes is present in the first tissue when the first plurality of sequences does not include a sequence spanning the junction of the respective gene fusion.

In some embodiments, the determining also includes determining, for each respective gene in the first set of genes within the plurality of genes, a second corresponding RNA boundary distribution for the respective gene comprising a corresponding relative abundance value for each respective RNA boundary sub-sequence in the plurality of RNA boundary sub-sequences of the respective gene using a second plurality of sequences, obtained in a second sequencing reaction, for a second plurality of mRNA molecules in a second biological sample of a second tissue of the test subject, and the evaluating also includes evaluating the second corresponding RNA boundary distribution for the first gene in the respective pair of genes and the second corresponding RNA boundary distribution for the second gene in the respective pair of genes from the determining (B) with the model. In some embodiments, second tissue of the subject is not the same type of tissue as the first tissue. In some embodiments, the first tissue of the subject is a cancerous tissue of the subject and the second tissue of the subject is a non-cancerous tissue of the subject.

In some embodiments, the model is trained specifically for determining a status for a gene fusion between a first respective pair of genes in the plurality of genes.

In some embodiments, the evaluating also evaluating a tissue type of the first tissue of the test subject using the model.

In some embodiments, the model is trained specifically for determining a status for a gene fusion in a tissue type of the first tissue of the test subject.

In some embodiments, the method also includes generating, based on the first plurality of sequences, a sample-specific distribution (e.g., a standard curve) of measures of relative abundance {e.g., a number of unique reads containing an RNA boundary sub-sequence for the gene} for mRNA molecules corresponding to each respective gene, in a second set of genes within the plurality of genes, in the first biological sample, and determining a respective measure of confidence for the respective indication by comparing (a) a measure of relative abundance for the respective gene fusion between the respective pair of genes in the first biological sample, to (b) the sample-specific distribution.

In some embodiments, the comparing the measure of relative abundance for the gene fusion between the respective pair of genes to the sample-specific distribution includes: determining a level of detection, at a first level of confidence, for an mRNA transcript in the first plurality of sequences, and comparing a level of support for the respective gene fusion between the respective pair of genes in the first plurality of sequences to the level of detection.

Sequencing Complexity Analysis

In some embodiments, the analysis of aligned sequence reads, e.g., in SAM or BAM format, includes evaluation of the complexity of a nucleic acid sequencing reaction, e.g., an RNA-seq reaction, using one or more boundary element interpretation algorithms 171. For example, FIG. 4C illustrates a workflow of an exemplary method 450 for evaluating the complexity of a nucleic acid sequencing reaction, e.g., to support clinical decision making in treating a disease or disorder, in accordance with some embodiments of the present disclosure.

An overview of methods for providing clinical support for personalized therapy is described above with reference to FIGS. 1-4D above. Below, systems and methods based on RNA boundary analysis are described for improving analysis of sequencing complexity, e.g., within the context of the methods and systems described above, are described with reference to FIGS. 4C and 7A-7D.

Many of the embodiments described below, in conjunction with FIGS. 4C and 7A-7D, relate to analyses performed using sequencing data for nucleic acid molecules obtained from samples of a subject. Generally, these embodiments are independent and, thus, not reliant upon any particular nucleic acid sequencing methods. However, in some embodiments, the methods described below include generating the sequencing data.

In one aspect, the disclosure provides a method 7000 for evaluating the nucleic acid complexity of a nucleic acid sequencing reaction. In some embodiments, all or part of the method is performed on a computer system having one or more processors and memory storing one or more programs for execution by the one or more processors, e.g., such as system 100.

In some embodiments, the method includes sequencing (7002) a first plurality of mRNA molecules from a sample of the subject, or cDNA molecules generated therefrom, thereby generating a first plurality of sequence reads for the first plurality of mRNA molecules. However, in some embodiments, the sequencing has already been performed, e.g., and the method begins with obtaining nucleic acid sequences, electronically, from the previously executed sequencing reaction.

In some embodiments, the sequencing reaction is a panel-targeted sequencing reaction that uses a plurality of nucleic acid capture probe species. In some embodiments, each respective nucleic acid probe species (e.g., all nucleic acid probes that align to the same subsequence of a respective target region) in the plurality of nucleic acid probe species aligns to a different subsequence of a respective target region of a reference construct for the species of the subject. For instance, in some embodiments, a first respective set of nucleic acid probes tiles (e.g., via overlapping or non-overlapping tiling) a respective genomic region, such as a gene. Thus, the nucleic acid probes in the set of probes bind to different subsequences of the genomic region.

In some embodiments, the plurality of nucleic acid probe species comprises at least 100, at least 500, at least 1000, at least 2000, at least 5000, at least 10,000, at least 50,000, at least 100,000, at least 500,000, at least 1,000,000, at least 2,500,000, or at least 5,000,000 nucleic acid probe species. In some embodiments, the plurality of nucleic acid probe species is no more than 10,000,000, no more than 1,000,000, no more than 500,000, no more than 100,000, no more than 50,000, no more than 10,000, no more than 5000, no more than 1000, or no more than 500 nucleic acid probe species. In some embodiments, the plurality of nucleic acid probe species is from 100 to 500, from 250 to 1000, from 1000 to 5000, from 1000 to 10,000,000, from 1,000,000 to 10,000,000, from 100 to 5,000,000, or from 100,000 to 500,000 nucleic acid probe species. In some embodiments, the plurality of nucleic acid probe species falls within another range starting no lower than 100 nucleic acid probe species and ending no higher than 10,000,000 nucleic acid probe species.

Additional embodiments for probes suitable for use in the present disclosure are further described in U.S. patent application Ser. No. 17/076,704, filed Oct. 21, 2020, the content of which is hereby incorporated by reference, in its entirety, for all purposes.

Method 7000 includes obtaining (7004), in electronic form, a first plurality of at least 100,000 nucleic acid sequences for a first plurality of mRNA molecules from a first biological sample, where each mRNA molecule in the first plurality of mRNA molecules corresponds to one or more genes in a plurality of genes.

In some embodiments, the first plurality nucleic acid sequences is at least 1,000,000 sequences (7006). In some embodiments, the first plurality of nucleic acid sequences is at least 5000 nucleic acid sequences, at least 10,000 nucleic acid sequences, at least 50,000 nucleic acid sequences, at least 100,000 nucleic acid sequences, at least 250,000 nucleic acid sequences, at least 500,000 nucleic acid sequences, at least 2,000,000 nucleic acid sequences, or more nucleic acid sequences. In some embodiments, the first plurality of nucleic acid sequences is no more than 10,000,000 nucleic acid sequences, no more than 5,000,000 nucleic acid sequences, no more than 2,500,000 nucleic acid sequences, no more than 1,000,000 nucleic acid sequences, nor more than 500,000 nucleic acid sequences, no more than 250,000 nucleic acid sequences, or less.

In some embodiments, the first plurality nucleic acid sequences is from 5000 to 10,000,000 nucleic acid sequences. In some embodiments, the first plurality nucleic acid sequences is from 5000 to 5,000,000 nucleic acid sequences. In some embodiments, the first plurality nucleic acid sequences is from 5000 to 2,500,000 nucleic acid sequences. In some embodiments, the first plurality nucleic acid sequences is from 5000 to 1,000,000 nucleic acid sequences. In some embodiments, the first plurality nucleic acid sequences is from 5000 to 500,000 nucleic acid sequences. In some embodiments, the first plurality nucleic acid sequences is from 5000 to 250,000 nucleic acid sequences. In some embodiments, the first plurality nucleic acid sequences is from 25,000 to 10,000,000 nucleic acid sequences. In some embodiments, the first plurality nucleic acid sequences is from 25,000 to 5,000,000 nucleic acid sequences. In some embodiments, the first plurality nucleic acid sequences is from 25,000 to 2,500,000 nucleic acid sequences. In some embodiments, the first plurality nucleic acid sequences is from 25,000 to 5,000,000 nucleic acid sequences. In some embodiments, the first plurality nucleic acid sequences is from 25,000 to 2,500,000 nucleic acid sequences. In some embodiments, the first plurality nucleic acid sequences is from 25,000 to 1,000,000 nucleic acid sequences. In some embodiments, the first plurality nucleic acid sequences is from 25,000 to 500,000 nucleic acid sequences. In some embodiments, the first plurality nucleic acid sequences is from 25,000 to 250,000 nucleic acid sequences. In some embodiments, the first plurality nucleic acid sequences is from 100,000 to 10,000,000 nucleic acid sequences. In some embodiments, the first plurality nucleic acid sequences is from 100,000 to 5,000,000 nucleic acid sequences. In some embodiments, the first plurality nucleic acid sequences is from 100,000 to 2,500,000 nucleic acid sequences. In some embodiments, the first plurality nucleic acid sequences is from 100,000 to 1,000,000 nucleic acid sequences. In some embodiments, the first plurality nucleic acid sequences is from 100,000 to 500,000 nucleic acid sequences. In some embodiments, the first plurality nucleic acid sequences is from 100,000 to 250,000 nucleic acid sequences. In some embodiments, the first plurality nucleic acid sequences is from 250,000 to 10,000,000 nucleic acid sequences. In some embodiments, the first plurality nucleic acid sequences is from 250,000 to 5,000,000 nucleic acid sequences. In some embodiments, the first plurality nucleic acid sequences is from 250,000 to 2,500,000 nucleic acid sequences. In some embodiments, the first plurality nucleic acid sequences is from 250,000 to 1,000,000 nucleic acid sequences. In some embodiments, the first plurality nucleic acid sequences is from 250,000 to 500,000 nucleic acid sequences.

In some embodiments, the one or more genes is at least 25 genes (7008). In some embodiments, the one or more genes is at least 10, at least 15, at least 25, at least 30, at least 40, at least 50, at least 100, at least 200, at least 250, at least 400, at least 500, at least 600, at least 700, at least 800, at least 900, at least 1000, at least 2000, at least 2500, at least 4000, at least 5000, at least 6000, at least 7000, at least 8000, at least 9000, at least 10,000, at least 15,000, or at least 20,000 genes. In some embodiments, the one or more genes is no more than 40,000, no more than 30,000, no more than 20,000, no more than 10,000, no more than 8000, no more than 7500, no more than 5000, no more than 4000, no more than 3000, no more than 2000, no more than 1000, no more than 750, no more than 500, no more than 250, no more than 100, no more than 50, or no more than 25 genes. In some embodiments, the one or more genes is from 10 to 50,000 genes. In some embodiments, the one or more genes is from 10 to 40,000 genes. In some embodiments, the one or more genes is from 10 to 25,000 genes. In some embodiments, the one or more genes is from 10 to 10,000 genes. In some embodiments, the one or more genes is from 10 to 5000 genes. In some embodiments, the one or more genes is from 10 to 1000 genes. In some embodiments, the one or more genes is from 10 to 500 genes. In some embodiments, the one or more genes is from 25 to 50,000 genes. In some embodiments, the one or more genes is from 25 to 40,000 genes. In some embodiments, the one or more genes is from 25 to 25,000 genes. In some embodiments, the one or more genes is from 25 to 10,000 genes. In some embodiments, the one or more genes is from 25 to 5000 genes. In some embodiments, the one or more genes is from 25 to 1000 genes. In some embodiments, the one or more genes is from 25 to 500 genes. In some embodiments, the one or more genes is from 100 to 50,000 genes. In some embodiments, the one or more genes is from 100 to 40,000 genes. In some embodiments, the one or more genes is from 100 to 25,000 genes. In some embodiments, the one or more genes is from 100 to 10,000 genes. In some embodiments, the one or more genes is from 100 to 5000 genes. In some embodiments, the one or more genes is from 100 to 1000 genes. In some embodiments, the one or more genes is from 100 to 500 genes. In some embodiments, the one or more genes is from 500 to 50,000 genes. In some embodiments, the one or more genes is from 500 to 40,000 genes. In some embodiments, the one or more genes is from 500 to 25,000 genes. In some embodiments, the one or more genes is from 500 to 10,000 genes. In some embodiments, the one or more genes is from 500 to 5000 genes. In some embodiments, the one or more genes is from 500 to 1000 genes. In some embodiments, the one or more genes is from 1000 to 50,000 genes. In some embodiments, the one or more genes is from 1000 to 40,000 genes. In some embodiments, the one or more genes is from 1000 to 25,000 genes. In some embodiments, the one or more genes is from 1000 to 10,000 genes. In some embodiments, the one or more genes is from 1000 to 5000 genes. In some embodiments, the one or more genes is from 5000 to 50,000 genes. In some embodiments, the one or more genes is from 5000 to 40,000 genes. In some embodiments, the one or more genes is from 5000 to 25,000 genes. In some embodiments, the one or more genes is from 5000 to 10,000 genes. In some embodiments, the one or more genes represents a whole transcriptome (7010).

In some embodiments, first plurality of nucleic acid sequences were obtained by sequencing cDNA. generated from the first plurality of mRNA molecules from the first biological sample (7012).

In some embodiments, the first biological sample of the subject is a solid tumor sample from the subject (7014). In some embodiments, the first biological sample of the subject is a non-cancerous tissue sample from the subject (7016). In some embodiments, the first biological sample of the subject is a saliva sample or a blood sample from the subject (7018). In some embodiments, the method is performed for more than one type of biological sample from the subject, e.g., for 2, 3, 4, 5, 6, 7, 8, 9, 10, or more biological samples from the subject.

Method 7000 also includes obtaining (7020) a first dataset by a process including determining, for each respective gene in a first set of genes within the first plurality of genes, one or more corresponding abundance values for RNA boundary elements of the respective gene in the first plurality of nucleic acid sequences. In some embodiments, the one or more corresponding abundance values for RNA boundary elements of the respective gene comprise one or more exon-exon boundaries for the respective gene (7022). In some embodiments, the process for obtaining the first dataset includes determining (7024), for each respective nucleic acid sequence in the first plurality of nucleic acid sequences, the respective one or more genes in the plurality of genes corresponding to the respective nucleic acid sequence by mapping the respective nucleic acid sequence to a reference construct for the species of the subject, identifying, for each respective nucleic acid sequence in the plurality of nucleic acids sequences that maps to a respective gene in the first set of genes, each RNA boundary element in the respective plurality of boundary elements that is present in the respective nucleic acid sequence, and counting, for each respective gene in the first set of genes, the number of occurrences of each respective RNA boundary element in the respective plurality of boundary elements across each respective nucleic acid sequence in the plurality of nucleic acid sequences that maps to a respective gene in the first set of genes, thereby generating a respective abundance value for each respective boundary element in the respective plurality of boundary elements.

In some embodiments, the reference construct represents at least 1 Mb of the genome, exome, and/or transcriptome for the species of the subject (7026). In other embodiments, the reference construct represents at least 250 kb, 500 kb, 750 kb, 1 Mb, 2 Mb, 5 Mb, 10 Mb, 25 Mb, 50 Mb, 100 Mb, 250 Mb, or more of the genome, exome, and/or transcriptome for the species of the subject. However, in some embodiments, there is no size limitation of the reference sequence. For example, in some embodiments, the reference sequence can be a sequence for a single locus, e.g., a single exon, gene, etc. within the genome, exome, and/or transcriptome for the species of the subject.

In some embodiments, the first set of genes is at least 25 genes (7028). In some embodiments, the first set of genes is at least 10, at least 15, at least 25, at least 30, at least 40, at least 50, at least 100, at least 200, at least 250, at least 400, at least 500, at least 600, at least 700, at least 800, at least 900, at least 1000, at least 2000, at least 2500, at least 4000, at least 5000, at least 6000, at least 7000, at least 8000, at least 9000, at least 10,000, at least 15,000, or at least 20,000 genes. In some embodiments, the first set of genes is no more than 40,000, no more than 30,000, no more than 20,000, no more than 10,000, no more than 8000, no more than 7500, no more than 5000, no more than 4000, no more than 3000, no more than 2000, no more than 1000, no more than 750, no more than 500, no more than 250, no more than 100, no more than 50, or no more than 25 genes. In some embodiments, the first set of genes is from 10 to 50,000 genes. In some embodiments, the first set of genes is from 10 to 40,000 genes. In some embodiments, the first set of genes is from 10 to 25,000 genes. In some embodiments, the first set of genes is from 10 to 10,000 genes. In some embodiments, the first set of genes is from 10 to 5000 genes. In some embodiments, the first set of genes is from 10 to 1000 genes. In some embodiments, the first set of genes is from 10 to 500 genes. In some embodiments, the first set of genes is from 25 to 50,000 genes. In some embodiments, the first set of genes is from 25 to 40,000 genes. In some embodiments, the first set of genes is from 25 to 25,000 genes. In some embodiments, the first set of genes is from 25 to 10,000 genes. In some embodiments, the first set of genes is from 25 to 5000 genes. In some embodiments, the first set of genes is from 25 to 1000 genes. In some embodiments, the first set of genes is from 25 to 500 genes. In some embodiments, the first set of genes is from 100 to 50,000 genes. In some embodiments, the first set of genes is from 100 to 40,000 genes. In some embodiments, the first set of genes is from 100 to 25,000 genes. In some embodiments, the first set of genes is from 100 to 10,000 genes. In some embodiments, the first set of genes is from 100 to 5000 genes. In some embodiments, the first set of genes is from 100 to 1000 genes. In some embodiments, the first set of genes is from 100 to 500 genes. In some embodiments, the first set of genes is from 500 to 50,000 genes. In some embodiments, the first set of genes is from 500 to 40,000 genes. In some embodiments, the first set of genes is from 500 to 25,000 genes. In some embodiments, the first set of genes is from 500 to 10,000 genes. In some embodiments, the first set of genes is from 500 to 5000 genes. In some embodiments, the first set of genes is from 500 to 1000 genes. In some embodiments, the first set of genes is from 1000 to 50,000 genes. In some embodiments, the first set of genes is from 1000 to 40,000 genes. In some embodiments, the first set of genes is from 1000 to 25,000 genes. In some embodiments, the first set of genes is from 1000 to 10,000 genes. In some embodiments, the first set of genes is from 1000 to 5000 genes. In some embodiments, the first set of genes is from 5000 to 50,000 genes. In some embodiments, the first set of genes is from 5000 to 40,000 genes. In some embodiments, the first set of genes is from 5000 to 25,000 genes. In some embodiments, the first set of genes is from 5000 to 10,000 genes. In some embodiments, the first set of genes represents a whole transcriptome (7030).

In some embodiments, corresponding abundance values are determined for each of at least 100 respective RNA boundary elements (7032). In some embodiments, corresponding abundance values are determined for a first set of RNA boundary elements. In some embodiments, the first set of RNA boundary elements is at least 10, at least 15, at least 25, at least 30, at least 40, at least 50, at least 100, at least 200, at least 250, at least 400, at least 500, at least 600, at least 700, at least 800, at least 900, at least 1000, at least 2000, at least 2500, at least 4000, at least 5000, at least 6000, at least 7000, at least 8000, at least 9000, at least 10,000, at least 15,000, at least 20,000, at least 50,000, at least 100,000, at least 250,000, at least 500,000, at least 1,000,000, or more RNA boundary elements. In some embodiments, the first set of RNA boundary elements is no more than 10,000,000, no more than 5,000,000, no more than 2,500,000, no more than 1,000,000, no more than 750,000, no more than 500,000, no more than 250,000, no more than 100,000, no more than 50,000, no more than 25,000, no more than 10,000, no more than 5000, or no more than 2500 RNA boundary elements. In some embodiments, the first set of RNA boundary elements is from 10 to 10,000,000 RNA boundary elements. In some embodiments, the first set of RNA boundary elements is from 10 to 5,000,000 RNA boundary elements. In some embodiments, the first set of RNA boundary elements is from 10 to 2,500,000 RNA boundary elements. In some embodiments, the first set of RNA boundary elements is from 10 to 1,000,000 RNA boundary elements. In some embodiments, the first set of RNA boundary elements is from 10 to 500,000 RNA boundary elements. In some embodiments, the first set of RNA boundary elements is from 10 to 100,000 RNA boundary elements. In some embodiments, the first set of RNA boundary elements is from 10 to 50,000 RNA boundary elements. In some embodiments, the first set of RNA boundary elements represents a whole transcriptome.

Method 7000 also includes applying (7034) a model to the first dataset, or a plurality of dimensionality reduction components thereof, thereby determining the genetic status of the subject as output of the model.

A variety of dimensionality reduction techniques can be used. Examples include, but are not limited to, principal component analysis (PCA), non-negative matrix factorization (NMF), linear discriminant analysis (LDA), diffusion maps, or network (e.g., neural network) techniques such as an autoencoder. In some embodiments, the dimension reduction is a principal components algorithm, a random projection algorithm, an independent component analysis algorithm, a feature selection method, a factor analysis algorithm, Sammon mapping, curvilinear components analysis, a stochastic neighbor embedding (SNE) algorithm, an Isomap algorithm, a maximum variance unfolding algorithm, a locally linear embedding algorithm, a t-SNE algorithm, a non-negative matrix factorization algorithm, a kernel principal component analysis algorithm, a graph-based kernel principal component analysis algorithm, a linear discriminant analysis algorithm, a generalized discriminant analysis algorithm, a uniform manifold approximation and projection (UMAP) algorithm, a LargeVis algorithm, a Laplacian Eigenmap algorithm, or a Fisher's linear discriminant analysis algorithm. See, for example, Fodor, 2002, “A survey of dimension reduction techniques,” Center for Applied Scientific Computing, Lawrence Livermore National, Technical Report UCRL-ID-148494; Cunningham, 2007, “Dimension Reduction,” University College Dublin, Technical Report UCD-CSI-2007-7, Zahorian et al., 2011, “Nonlinear Dimensionality Reduction Methods for Use with Automatic Speech Recognition,” Speech Technologies. doi:10.5772/16863. ISBN 978-953-307-996-7; and Lakshmi et al., 2016, “2016 IEEE 6th International Conference on Advanced Computing (IACC),” pp. 31-34. doi:10.1109/IACC.2016.16, ISBN 978-1-4673-8286-1, the contents of which are hereby incorporated by reference, in their entireties, for all purposes. Accordingly, in some embodiments, the dimension reduction is a principal component analysis (PCA) algorithm, and each respective extracted dimension reduction component comprises a respective principal component derived by the PCA. In such embodiments, the number of principal components in the plurality of principal components can be limited to a threshold number of principal components calculated by the PCA algorithm. The threshold number of principal components can be, for example, at least 5, at least 10, at least 20, at least 50, at least 100, at least 1000, at least 1500, or any other number. In some embodiments, the method further includes performing manifold learning using the dataset, e.g., as described above with respect to method 5000.

In some embodiments, the measure of the nucleic acid complexity is a level of detection, in the nucleic acid sequencing reaction, for a respective species of mRNA molecule present in the first biological sample (7036). In some embodiments, the measure of the nucleic acid complexity is a confidence value that a respective species of mRNA molecule detected in the first plurality of nucleic acid sequences is present in the first biological sample (7038). In some embodiments, the measure of the nucleic acid complexity is a confidence value that a respective species of mRNA molecule not detected in the first plurality of nucleic acid sequences is not present in the first biological sample (7040). In some embodiments, the measure of the nucleic acid complexity is an estimate of the number of mRNA molecules, from the first biological sample, represented in the first plurality of nucleic acid sequences (7042).

In some embodiments, the model is a statistical inference model (7044). In some embodiments, the statistical inference model is a Bayesian inference model, a likelihood-based inference model, a frequentist inference model, or an AIC-based inference model (7046). In some embodiments, the statistical inference model is a mixture model (7048). In some embodiments, the statistical inference comprises any suitable statistical inference model disclosed herein, and/or any combinations, modifications, substitutions, additions, or deletions thereof as will be apparent to one skilled in the art (see, e.g., the section entitled, “Definitions: Classifier,” above).

In some embodiments, the model is a machine learning model (7050). In some embodiments, the machine learning model is a support vector regression, a random forest model, an XGBoost model, a Gaussian process model, a deep neural network model, a convolutional neural network model, or a recurrent neural network model (7052). In some embodiments, the machine learning model comprises any suitable machine learning model disclosed herein, and/or any combinations, modifications, substitutions, additions, or deletions thereof as will be apparent to one skilled in the art (see, e.g., the section entitled, “Definitions: Classifier,” above).

In some embodiments, the model is a regression model (7054). In some embodiments, the regression model comprises any suitable regression model disclosed herein, and/or any combinations, modifications, substitutions, additions, or deletions thereof as will be apparent to one skilled in the art (see, e.g., the section entitled, “Definitions: Classifier,” above).

In some embodiments, the model processes the first data set, or a plurality of dimensionality reduction components thereof, to determine the genetic status of the subject as an output of the model in N-dimensional space in the applying (7056), where N is a positive integer of at least 4. In some embodiments, N is 5, 6, 7, 8, 9, 10, 11, 12, 13, 14, 15, 20, 50, 100, 1000, 10,000, 100,000, 500,000, 1×10⁶, 5×10⁶, 1×10⁷, or greater.

In some embodiments, the model comprises a plurality of at least 500 parameters. In some embodiments, the plurality of parameters comprises at least 10, at least 50, at least 100, at least 500, at least 1000, at least 2000, at least 5000, at least 10,000, at least 20,000, at least 50,000, at least 100,000, at least 200,000, at least 500,000, at least 1 million, at least 2 million, at least 3 million, at least 4 million or at least 5 million parameters. In some embodiments, the plurality of parameters comprises no more than 8 million, no more than 5 million, no more than 4 million, no more than 1 million, no more than 500,000, no more than 100,000, no more than 50,000, no more than 10,000, no more than 5000, no more than 1000, or no more than 500 parameters. In some embodiments, the plurality of parameters comprises from 10 to 5000, from 500 to 10,000, from 10,000 to 500,000, from 20,000 to 1 million, or from 1 million to 5 million parameters. In some embodiments, the plurality of parameters falls within another range starting no lower than 10 parameters and ending no higher than 8 million parameters.

In some embodiments, the model used in method 7000 can be tumor specific, fusion-specific, and/or cancer specific. That is, in some embodiments, the model is trained to evaluate the complexity of the sequencing reaction when the sample is a sample of a particular type of tumor. Similarly, in some embodiments, the model is trained to evaluate the complexity of the sequencing reaction when trying to determine whether a particular gene fusion is present in the sample. Similarly, in some embodiments, the model is trained to evaluate the complexity of the sequencing reaction when the sample is a sample of a particular type of cancer.

In some embodiments, the model is a dynamic model of RNA species, e.g., RNA fusion, detection, which provides a higher probability of detecting an RNA species, e.g., an RNA fusions, by determining whether the sequencing coverage in the reaction is high or low. In some embodiments, the model provides a warning that the sequencing reaction will have low sensitivity when the sequencing reaction has low sequence coverage, as determined by RNA boundary analysis. In some embodiments, the depth and breadth of an RNA boundary profile determined for a sequencing reaction provides a measure of RNA transcriptome integrity in the sequencing reaction. When the depth and/or breadth of the RNA boundary profile is determined to be low, there is a greater likelihood of not detecting a particular RNA species that is present in the tissue of the subject be tested.

That is, when a particular RNA species, e.g., an isoform variant or gene fusion, is not detected in an RNA sequencing reaction, there are two possible explanations. Either the RNA species is really not present in the transcriptome of the tissue being sampled or the coverage of the RNA sequencing reaction was too low, e.g., because of low complexity in the nucleic acid sample or poor quality of the nucleic acid sample. The methods and system provided herein assist in determining which of these possibilities leads to the non-identification of the mRNA species. In this fashion, a confidence can be assigned to the absence of a particular mRNA species, e.g., a higher confidence that the subject does not have the species when the RNA boundary profile is more robust and a lower confidence when the RNA boundary profile is less robust. In some embodiments, the expectation for the RAN boundary profile is dependent upon one or more characteristic of the tissue sample and/or one or more personal characteristic of the subject.

Example Methods for Analyzing Sequencing Complexity

In one aspect, the disclosure provides methods and systems for determining a level of detection for an mRNA species, e.g., a wild-type transcript or splice variant thereof, a transcript containing an indel, a transcript of a gene fusion, a transcript of a gene rearrangement, etc., in biological sample of a test subject. The method includes, on a computer system having one or more processors, and memory storing one or more programs for execution by the one or more processors, obtaining, from a first sequencing reaction, a plurality of mRNA sequences for a plurality of mRNA molecules in the biological sample from the test subject, where each mRNA molecule in the plurality of mRNA molecules belongs to a respective species of mRNA molecule in a plurality of species of mRNA molecules. The method also includes quantifying, for each respective species of mRNA molecule in the plurality of species of mRNA molecules, a respective relative abundance of corresponding sequences of mRNA molecules belonging to the respective species of mRNA molecule in the plurality of mRNA sequences by evaluating a quantity of RNA boundary sub-sequences corresponding to the respective species of mRNA molecule present in the plurality of RNA sequences. Generally, there are a few levels to the data: one is gene level transcripts, which will generally have a consistent relative abundance for a given tissue type expression level, the other is boundary reads (e.g., exon-exon boundaries, gene fusion boundaries, other genomic rearrangement boundaries (e.g., insertions, deletions, and translocations) obtained for each gene. Thereby, the method generates a distribution of relative abundance values for the plurality of species of mRNA molecules in the biological sample. The method also includes evaluating one or more respective relative abundance values in the distribution of relative abundance values using a model trained to estimate the mRNA level of detection for a sample based on the relative abundance values for a set of species of mRNA molecules in a sequencing reaction, thereby determining the level of detection for an mRNA species in the first sequencing reaction.

In some embodiments, the method also includes determining a measure of confidence for the detection, in the first sequencing reaction, of a rare mRNA species present at a first relative abundance in the biological sample from the test subject based on at least the level of detection estimated for the sample.

In some embodiments, the measure of confidence is a sensitivity for the detection, in the first sequencing reaction, of the rare mRNA species. In some embodiments, the rare mRNA species is a gene fusion transcript. In some embodiments, the rare mRNA species is a splice variant transcript.

In some embodiments, the plurality of mRNA sequences is at least 10,000 mRNA sequences. In some embodiments, the plurality of mRNA sequences is at least 5000, at least 10,000, at least 25,000, at least 50,000, at least 100,000, at least 250,000, at least 500,000, at least 1,000,000, or more mRNA sequences.

In some embodiments, the plurality of species of mRNA molecules is at least 50 species of mRNA molecules. In some embodiments, the plurality of species of mRNA molecules is at least 10, at least 25, at least 50, at least 100, at least 250, at least 500, at least 1000, at least 2500, at least 5000, at least 10,000, at least 25,000, or more species of mRNA.

In one aspect, the disclosure provides a method of evaluating a transcriptome of a test subject. The method includes performing a first sequencing reaction on a first amount of cDNA generated from RNA isolated from a biological sample of the test subject, thereby obtaining a first plurality of mRNA sequences for a plurality of mRNA molecules in the biological sample, where each mRNA molecule in the plurality of mRNA molecules belongs to a respective species of mRNA molecule in a plurality of species of mRNA molecules. The method also includes determining a measure of mRNA diversity in the first sequencing reaction by: (i) quantifying, for each respective species of mRNA molecule in the plurality of species of mRNA molecules, a respective relative abundance of corresponding sequences of mRNA molecules belonging to the respective species of mRNA molecule in the first plurality of mRNA sequences by evaluating a quantity of RNA boundary sub-sequences corresponding to the respective species of mRNA molecule present in the first plurality of RNA sequences, thereby generating a distribution of relative abundance values for the plurality of species of mRNA molecules in the biological sample, and (ii) evaluating one or more respective relative abundance values in the distribution of relative abundance values using a model trained to estimate a measure of mRNA diversity for a sample based on the relative abundance values for a set of species of mRNA molecules in a sequencing reaction, thereby determining the measure of mRNA diversity for the first sequencing reaction. The method also includes determining whether the measure of mRNA diversity in the first sequencing reaction satisfies a first threshold for mRNA diversity in a sequencing reaction, and when the measure of mRNA diversity in the first sequencing reaction satisfies the first threshold for mRNA diversity in a sequencing reaction, evaluating the first plurality of mRNA sequences obtained from the first sequencing reaction to determine one or more properties of the transcriptome of the test subject, and when the measure of the mRNA diversity in the first sequencing reaction does not satisfy the first threshold for mRNA diversity in a sequencing reaction: (a) performing a second sequencing reaction on a second amount of cDNA generated from RNA isolated from a biological sample of the test subject, thereby obtaining a second plurality of mRNA sequences for a plurality of mRNA molecules in the biological sample, wherein the second amount of cDNA is greater than the first amount of cDNA, and (b) evaluating the second plurality of mRNA sequences obtained from the second sequencing reaction to determine one or more properties of the transcriptome of the test subject.

In some embodiments, the measure of mRNA diversity for the first sequencing reaction is a level of detection for an mRNA species in the first sequencing reaction. In some embodiments, the measure of mRNA diversity for the first sequencing reaction is a sensitivity for the detection of a rare mRNA species in the first sequencing reaction.

In some embodiments, the second amount of cDNA is determined by inputting the measure of mRNA diversity into an algorithm trained to extrapolate an amount of cDNA necessary to achieve a desired measure of mRNA diversity in a sequencing reaction given the measure of mRNA diversity achieved using the first amount of cDNA.

In some embodiments, the plurality of mRNA sequences is at least 10,000 mRNA sequences. In some embodiments, the plurality of mRNA sequences is at least 5000, at least 10,000, at least 25,000, at least 50,000, at least 100,000, at least 250,000, at least 500,000, at least 1,000,000, or more mRNA sequences.

In some embodiments, the plurality of species of mRNA molecules is at least 50 species of mRNA molecules. In some embodiments, the plurality of species of mRNA molecules is at least 10, at least 25, at least 50, at least 100, at least 250, at least 500, at least 1000, at least 2500, at least 5000, at least 10,000, at least 25,000, or more species of mRNA.

Digital and Laboratory Health Care Platform

In some embodiments, the methods and systems described herein are utilized in combination with, or as part of, a digital and laboratory health care platform that is generally targeted to medical care and research. It should be understood that many uses of the methods and systems described above, in combination with such a platform, are possible. One example of such a platform is described in U.S. Patent Publication No. 2021/0090694, titled “Data Based Cancer Research and Treatment Systems and Methods”, and published Mar. 25, 2021, the content of which is incorporated herein by reference, in its entirety, for all purposes.

For example, an implementation of one or more embodiments of the methods and systems as described above may include microservices constituting a digital and laboratory health care platform supporting analysis of cancer biopsy samples to provide clinical support for personalized cancer therapy. Embodiments may include a single microservice for executing and delivering analysis of cancer biopsy samples to clinical support for personalized cancer therapy or may include a plurality of microservices each having a particular role, which together implement one or more of the embodiments above. In one example, a first microservice may execute sequence analysis in order to deliver genomic features to a second microservice for curating clinical support for personalized cancer therapy based on the identified features. Similarly, the second microservice may execute therapeutic analysis of the curated clinical support to deliver recommended therapeutic modalities, according to various embodiments described herein.

Where embodiments above are executed in one or more micro-services with or as part of a digital and laboratory health care platform, one or more of such micro-services may be part of an order management system that orchestrates the sequence of events as needed at the appropriate time and in the appropriate order necessary to instantiate embodiments above. A microservices-based order management system is disclosed, for example, in U.S. Patent Publication No. 2020/80365232, titled “Adaptive Order Fulfillment and Tracking Methods and Systems”, and published Nov. 19, 2020, the content of which is incorporated herein by reference, in its entirety, for all purposes.

For example, continuing with the above first and second microservices, an order management system may notify the first microservice that an order for curating clinical support for personalized cancer therapy has been received and is ready for processing. The first microservice may execute and notify the order management system once the delivery of genomic features for the patient is ready for the second microservice. Furthermore, the order management system may identify that execution parameters (prerequisites) for the second microservice are satisfied, including that the first microservice has completed, and notify the second microservice that it may continue processing the order to curate clinical support for personalized cancer therapy, according to various embodiments described herein.

Where the digital and laboratory health care platform further includes a genetic analyzer system, the genetic analyzer system may include targeted panels and/or sequencing probes. An example of a targeted panel is disclosed, for example, in U.S. Patent Publication No. 2021/0090694, titled “Data Based Cancer Research and Treatment Systems and Methods”, and published Mar. 25, 2021, which is incorporated herein by reference and in its entirety for all purposes. An example of a targeted panel for sequencing cell-free (cf) DNA and determining various characteristics of a specimen based on the sequencing is disclosed, for example, in U.S. patent application Ser. No. 17/179,086, titled “Methods And Systems For Dynamic Variant Thresholding In A Liquid Biopsy Assay”, and filed Feb. 18, 1921, U.S. patent application Ser. No. 17/179,267, titled “Estimation Of Circulating Tumor Fraction Using Off-Target Reads Of Targeted-Panel Sequencing”, and filed Feb. 18, 1921, and U.S. patent application Ser. No. 17/179,279, titled “Methods And Systems For Refining Copy Number Variation In A Liquid Biopsy Assay”, and filed Feb. 18, 1921 which is incorporated herein by reference and in its entirety for all purposes. In one example, targeted panels may enable the delivery of next generation sequencing results for providing clinical support for personalized cancer therapy according to various embodiments described herein. An example of the design of next-generation sequencing probes is disclosed, for example, in U.S. Patent Publication No. 2021/0115511, titled “Systems and Methods for Next Generation Sequencing Uniform Probe Design”, and published Jun. 22, 2021 and U.S. patent application Ser. No. 17/323,986, titled “Systems and Methods for Next Generation Sequencing Uniform Probe Design”, and filed May 18, 2021, which is incorporated herein by reference and in its entirety for all purposes.

Where the digital and laboratory health care platform further includes an epigenetic analyzer system, the epigenetic analyzer system may analyze specimens to determine their epigenetic characteristics and may further use that information for monitoring a patient over time. An example of an epigenetic analyzer system is disclosed, for example, in U.S. patent application Ser. No. 17/352,231, titled “Molecular Response And Progression Detection From Circulating Cell Free DNA”, and filed Jun. 18, 1921, which is incorporated herein by reference and in its entirety for all purposes.

Where the digital and laboratory health care platform further includes a bioinformatics pipeline, the methods and systems described above may be utilized after completion or substantial completion of the systems and methods utilized in the bioinformatics pipeline. As one example, the bioinformatics pipeline may receive next-generation genetic sequencing results and return a set of binary files, such as one or more BAM files, reflecting nucleic acid (e.g., cfDNA, DNA and/or RNA) read counts aligned to a reference genome. The methods and systems described above may be utilized, for example, to ingest the cfDNA, DNA and/or RNA read counts and produce genomic features as a result.

When the digital and laboratory health care platform further includes an RNA data normalizer, any RNA read counts may be normalized before processing embodiments as described above. An example of an RNA data normalizer is disclosed, for example, in Publication No. 2020/0098448, titled “Methods of Normalizing and Correcting RNA Expression Data”, and published Mar. 26, 2020, which is incorporated herein by reference and in its entirety for all purposes.

When the digital and laboratory health care platform further includes a genetic data deconvolver, any system and method for deconvoluting may be utilized for analyzing genetic data associated with a specimen having two or more biological components to determine the contribution of each component to the genetic data and/or determine what genetic data would be associated with any component of the specimen if it were purified. An example of a genetic data deconvolver is disclosed, for example, in U.S. Patent Publication No. 2020/0210852, published Jul. 2, 2020, and PCT/US19/69161, filed Dec. 31, 2019, both titled “Transcriptome Deconvolution of Metastatic Tissue Samples”; and U.S. patent application Ser. No. 17/074,984, titled “Calculating Cell-type RNA Profiles for Diagnosis and Treatment”, and filed Oct. 20, 2020, the content of each of which is incorporated herein by reference, in its entirety, for all purposes.

When the digital and laboratory health care platform further includes an automated RNA expression caller, RNA expression levels may be adjusted to be expressed as a value relative to a reference expression level, which is often done in order to prepare multiple RNA expression data sets for analysis to avoid artifacts caused when the data sets have differences because they have not been generated by using the same methods, equipment, and/or reagents. An example of an automated RNA expression caller is disclosed, for example, in U.S. Pat. No. 11,043,283, titled “Systems and Methods for Automating RNA Expression Calls in a Cancer Prediction Pipeline”, and issued Jun. 22, 2021, which is incorporated herein by reference and in its entirety for all purposes.

RNA expression levels may be adjusted to be expressed as a value relative to a reference expression level. Furthermore, multiple RNA expression data sets may be adjusted, prepared, and/or combined for analysis and may be adjusted to avoid artifacts caused when the data sets have differences because they have not been generated by using the same methods, equipment, and/or reagents. An example of RNA data set adjustment, preparation, and/or combination is disclosed, for example, in U.S. patent application Ser. No. 17/405,025, titled “Systems and Methods for Homogenization of Disparate Datasets”, and filed Aug. 18, 2021.

The digital and laboratory health care platform may further include one or more insight engines to deliver information, characteristics, or determinations related to a disease state that may be based on genetic and/or clinical data associated with a patient and/or specimen. Exemplary insight engines may include a tumor of unknown origin engine, a human leukocyte antigen (HLA) loss of homozygosity (LOH) engine, a tumor mutational burden engine, a PD-L1 status engine, a homologous recombination deficiency engine, a cellular pathway activation report engine, an immune infiltration engine, a microsatellite instability engine, a pathogen infection status engine, a T cell receptor or B cell receptor profiling engine, a line of therapy engine, a metastatic prediction engine, an IO progression risk prediction engine, and so forth. An example tumor of unknown origin engine is disclosed, for example, in U.S. patent application Ser. No. 15/930,234, titled “Systems and Methods for Multi-Label Cancer Classification”, and filed May 12, 2020, which is incorporated herein by reference and in its entirety for all purposes. An example of an HLA LOH engine is disclosed, for example, in U.S. Pat. No. 11,081,210, titled “Detection of Human Leukocyte Antigen Class I Loss of Heterozygosity in Solid Tumor Types by NGS DNA Sequencing”, and issued Aug. 3, 2021, which is incorporated herein by reference and in its entirety for all purposes. An additional example of an HLA LOH engine is disclosed, for example, in U.S. patent application Ser. No. 17/304,940, titled “Detection of Human Leukocyte Antigen Loss of Heterozygosity”, and filed Jun. 28, 2021, which is incorporated herein by reference and in its entirety for all purposes. An example of a tumor mutational burden (TMB) engine is disclosed, for example, in U.S. Patent Publication No. 2020/0258601, titled “Targeted-Panel Tumor Mutational Burden Calculation Systems and Methods”, and published Aug. 13, 2020, which is incorporated herein by reference and in its entirety for all purposes. An example of a PD-L1 status engine is disclosed, for example, in U.S. Patent Publication No. 2020/0395097, titled “A Pan-Cancer Model to Predict The PD-L1 Status of a Cancer Cell Sample Using RNA Expression Data and Other Patient Data”, and published Dec. 17, 2020, which is incorporated herein by reference and in its entirety for all purposes. An additional example of a PD-L1 status engine is disclosed, for example, in U.S. Pat. No. 10,957,041, titled “Determining Biomarkers from Histopathology Slide Images”, issued Mar. 23, 2021, which is incorporated herein by reference and in its entirety for all purposes. An example of a homologous recombination deficiency engine is disclosed, for example, in U.S. Pat. No. 10,975,445, titled “An Integrative Machine-Learning Framework to Predict Homologous Recombination Deficiency”, and issued Apr. 13, 2021, which is incorporated herein by reference and in its entirety for all purposes. An additional example of a homologous recombination deficiency engine is disclosed, for example, in U.S. patent application Ser. No. 17/492,518, titled “Systems and Methods for Predicting Homologous Recombination Deficiency Status of a Specimen”, filed Oct. 1, 2021, which is incorporated herein by reference and in its entirety for all purposes. An example of a cellular pathway activation report engine is disclosed, for example, in U.S. Patent Publication No. 2021/0057042, titled “Systems And Methods For Detecting Cellular Pathway Dysregulation In Cancer Specimens”, and published Feb. 25, 2021, which is incorporated herein by reference and in its entirety for all purposes. An example of an immune infiltration engine is disclosed, for example, in U.S. Patent Publication No. 2020/0075169, titled “A Multi-Modal Approach to Predicting Immune Infiltration Based on Integrated RNA Expression and Imaging Features”, and published Mar. 5, 2020, which is incorporated herein by reference and in its entirety for all purposes. An example of an MSI engine is disclosed, for example, in U.S. Patent Publication No. 2020/0118644, titled “Microsatellite Instability Determination System and Related Methods”, and published Apr. 16, 2020, which is incorporated herein by reference and in its entirety for all purposes. An additional example of an MSI engine is disclosed, for example, in U.S. Patent Publication No. 2021/0098078, titled “Systems and Methods for Detecting Microsatellite Instability of a Cancer Using a Liquid Biopsy”, and published Apr. 1, 2021, which is incorporated herein by reference and in its entirety for all purposes. An example of a pathogen infection status engine is disclosed, for example, in U.S. Pat. No. 11,043,304, titled “Systems And Methods For Using Sequencing Data For Pathogen Detection”, and issued Jun. 22, 2021, which is incorporated herein by reference and in its entirety for all purposes. Another example of a pathogen infection status engine is disclosed, for example, in PCT/US21/18619, titled “Systems And Methods For Detecting Viral DNA From Sequencing”, and filed Feb. 18, 2021, which is incorporated herein by reference and in its entirety for all purposes. An example of a T cell receptor or B cell receptor profiling engine is disclosed, for example, in U.S. patent application Ser. No. 17/302,030, titled “TCR/BCR Profiling Using Enrichment with Pools of Capture Probes”, and filed Apr. 21, 2021, which is incorporated herein by reference and in its entirety for all purposes. An example of a line of therapy engine is disclosed, for example, in U.S. Patent Publication No. 2021/0057071, titled “Unsupervised Learning And Prediction Of Lines Of Therapy From High-Dimensional Longitudinal Medications Data”, and published Feb. 25, 2021, which is incorporated herein by reference and in its entirety for all purposes. An example of a metastatic prediction engine is disclosed, for example, in U.S. Pat. No. 11,145,416, titled “Predicting likelihood and site of metastasis from patient records”, and issued Oct. 12, 2021, which is incorporated herein by reference and in its entirety for all purposes. An example of an IO progression risk prediction engine is disclosed, for example, in U.S. patent application Ser. No. 17/455,876, titled “Determination of Cytotoxic Gene Signature and Associated Systems and Methods For Response Prediction and Treatment”, and filed Nov. 19, 2021, which is incorporated herein by reference and in its entirety for all purposes.

Any data generated by the systems and methods and/or the digital and laboratory health care platform may be downloaded by the user. In one example, the data may be downloaded as a CSV file comprising clinical and/or molecular data associated with tests, data structuring, and/or other services ordered by the user. In various embodiments, this may be accomplished by aggregating clinical data in a system backend, and making it available via a portal. This data may include not only variants and RNA expression data, but also data associated with immunotherapy markers such as MSI and TMB, as well as RNA fusions.

When the digital and laboratory health care platform further includes a device comprising a microphone and speaker for receiving audible queries or instructions from a user and delivering answers or other information, the methods and systems described above may be utilized to add data to a database the device can access. An example of such a device is disclosed, for example, in U.S. Patent Publication No. 2020/0335102, titled “Collaborative Artificial Intelligence Method And System”, and published Oct. 22, 2020, which is incorporated herein by reference and in its entirety for all purposes.

When the digital and laboratory health care platform further includes a mobile application for ingesting patient records, including genomic sequencing records and/or results even if they were not generated by the same digital and laboratory health care platform, the methods and systems described above may be utilized to receive ingested patient records. An example of such a mobile application is disclosed, for example, in U.S. Pat. No. 10,395,772, titled “Mobile Supplementation, Extraction, And Analysis Of Health Records”, and issued Aug. 27, 2019, which is incorporated herein by reference and in its entirety for all purposes. Another example of such a mobile application is disclosed, for example, in U.S. Pat. No. 10,902,952, titled “Mobile Supplementation, Extraction, And Analysis Of Health Records”, and issued Jan. 26, 2021, which is incorporated herein by reference and in its entirety for all purposes. Another example of such a mobile application is disclosed, for example, in U.S. Patent Publication No. 2021/0151192, titled “Mobile Supplementation, Extraction, And Analysis Of Health Records”, and filed May 20, 2021, which is incorporated herein by reference and in its entirety for all purposes.

When the digital and laboratory health care platform further includes a report generation engine, the methods and systems described above may be utilized to create a summary report of a patient's genetic profile and the results of one or more insight engines for presentation to a physician. For instance, the report may provide to the physician information about the extent to which the specimen that was sequenced contained tumor or normal tissue from a first organ, a second organ, a third organ, and so forth. For example, the report may provide a genetic profile for each of the tissue types, tumors, or organs in the specimen. The genetic profile may represent genetic sequences present in the tissue type, tumor, or organ and may include variants, expression levels, information about gene products, or other information that could be derived from genetic analysis of a tissue, tumor, or organ.

The report may include therapies and/or clinical trials matched based on a portion or all of the genetic profile or insight engine findings and summaries. For example, the therapies may be matched according to the systems and methods disclosed in U.S. patent application Ser. No. 17/546,049, titled “Artificial Intelligence Driven Therapy Curation and Prioritization”, filed Dec. 9, 2021, which is incorporated herein by reference and in its entirety for all purposes. For example, the clinical trials may be matched according to the systems and methods disclosed in U.S. Patent Publication No. 2020/0381087, titled “Systems and Methods of Clinical Trial Evaluation”, published Dec. 3, 2020, which is incorporated herein by reference and in its entirety for all purposes.

The report may include a comparison of the results (for example, molecular and/or clinical patient data) to a database of results from many specimens. An example of methods and systems for comparing results to a database of results are disclosed in U.S. Patent Publication No. 2020/0135303 titled “User Interface, System, And Method For Cohort Analysis” and published Apr. 30, 2020, and U.S. Patent Publication No. 2020/0211716 titled “A Method and Process for Predicting and Analyzing Patient Cohort Response, Progression and Survival”, and published Jul. 2, 2020, which is incorporated herein by reference and in its entirety for all purposes. The information may be used, sometimes in conjunction with similar information from additional specimens and/or clinical response information, to match therapies likely to be successful in treating a patient, discover biomarkers or design a clinical trial.

When the digital and laboratory health care platform further includes organoids developed in connection with the platform (for example, from the patient specimen), the methods and systems may be used to further evaluate genetic sequencing data derived from an organoid and/or the organoid sensitivity, especially to therapies matched based on a portion or all of the information determined by the systems and methods, including predicted cancer type(s), likely tumor origin(s), etc. These therapies may be tested on the organoid, derivatives of that organoid, and/or similar organoids to determine an organoid's sensitivity to those therapies. Any of the results may be included in a report. If the organoid is associated with a patient specimen, any of the results may be included in a report associated with that patient and/or delivered to the patient or patient's physician or clinician. In various examples, organoids may be cultured and tested according to the systems and methods disclosed in U.S. Patent Publication No. 2021/0155989, titled “Tumor Organoid Culture Compositions, Systems, and Methods”, published May 27, 2021; PCT/US20/56930, titled “Systems and Methods for Predicting Therapeutic Sensitivity”, filed Oct. 22, 2020; U.S. Patent Publication No. 2021/0172931, titled “Large Scale Organoid Analysis”, published Jun. 10, 2021; PCT/US2020/063619, titled “Systems and Methods for High Throughput Drug Screening”, filed Dec. 7, 2020 and U.S. patent application Ser. No. 17/301,975, titled “Artificial Fluorescent Image Systems and Methods”, filed Apr. 20, 2021 which are each incorporated herein by reference and in their entirety for all purposes. In one example, the drug sensitivity assays may be especially informative if the systems and methods return results that match with a variety of therapies, or multiple results (for example, multiple equally or similarly likely cancer types or tumor origins), each matching with at least one therapy.

When the digital and laboratory health care platform further includes application of one or more of the above in combination with or as part of a medical device or a laboratory developed test that is generally targeted to medical care and research, such laboratory developed test or medical device results may be enhanced and personalized through the use of artificial intelligence. An example of laboratory developed tests, especially those that may be enhanced by artificial intelligence, is disclosed, for example, in U.S. Patent Publication No. 2021/0118559, titled “Artificial Intelligence Assisted Precision Medicine Enhancements to Standardized Laboratory Diagnostic Testing”, and published Apr. 22, 2021, which is incorporated herein by reference and in its entirety for all purposes.

It should be understood that the examples given above are illustrative and do not limit the uses of the systems and methods described herein in combination with a digital and laboratory health care platform.

The results of the bioinformatics pipeline may be provided for report generation 208. Report generation may comprise variant science analysis, including the interpretation of variants (including somatic and germline variants as applicable) for pathogenic and biological significance. The variant science analysis may also estimate microsatellite instability (MSI) or tumor mutational burden. Targeted treatments may be identified based on gene, variant, and cancer type, for further consideration and review by the ordering physician. In some aspects, clinical trials may be identified for which the patient may be eligible, based on mutations, cancer type, and/or clinical history. Subsequent validation may occur, after which the report may be finalized for sign-out and delivery. In some embodiments, a first or second report may include additional data provided through a clinical dataflow 202, such as patient progress notes, pathology reports, imaging reports, and other relevant documents. Such clinical data is ingested, reviewed, and abstracted based on a predefined set of curation rules. The clinical data is then populated into the patient's clinical history timeline for report generation.

Further details on clinical report generation are disclosed in U.S. patent application Ser. No. 16/789,363 (PCT/US20/180002), filed Feb. 12, 2020, the content of which is incorporated herein by reference, in its entirety, for all purposes.

The implementations provided herein are chosen and described in order to best explain the principles and their practical applications, to thereby enable others skilled in the art to best utilize the various embodiments with various modifications as are suited to the particular use contemplated. In some instances, well-known methods, procedures, components, circuits, and networks have not been described in detail so as not to unnecessarily obscure aspects of the embodiments. In other instances, it will be apparent to one of ordinary skill in the art that the present disclosure may be practiced without one or more of the specific details.

It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that though such a design effort might be complex and time-consuming, it will nevertheless be a routine undertaking of engineering for those of ordering skill in the art having the benefit of the present disclosure.

### References Cited and Alternative Embodiments

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A method for determining a genetic status of a subject, comprising:
on a computer system having one or more processors, and memory storing one or more programs for execution by the one or more processors:
A) obtaining, in electronic form, a first plurality of at least 100,000 nucleic acid sequences for a first plurality of mRNA molecules from a first biological sample of the subject, wherein each mRNA molecule in the first plurality of mRNA molecules corresponds to one or more genes in a plurality of genes;
B) obtaining a first dataset by a process comprising determining, for each respective gene in a first set of genes within the first plurality of genes, a corresponding abundance value for each respective RNA boundary element in a respective plurality of boundary elements of the respective gene in the first plurality of nucleic acid sequences; and
C) applying a model to the first dataset, or a plurality of dimensionality reduction components thereof, thereby determining the genetic status of the subject as output of the model.

2. The method of claim 1, wherein:
the first set of genes comprises a first respective gene in the plurality of genes;
the respective plurality of boundary elements comprises each exon-exon boundary present in at least one respective mRNA isoform in a plurality of mRNA isoforms for the respective gene; and
the genetic status of the subject comprises an mRNA isoform status for the first respective gene.

3. The method of claim 2, wherein:
the mRNA isoform status for the first respective gene (i) comprises an indication of whether the subject has a particular splicing pattern for the first respective gene, or (ii) is an estimate of the prevalence, in the first plurality of mRNA molecules, of one or more respective mRNA isoform in the plurality of mRNA isoforms, and:
the respective plurality of boundary elements further comprises a boundary element for a genomic rearrangement contained entirely within the first respective gene.

4-7. (canceled)

8. The method of claim 2, wherein:
the subject has a disease or disorder; and
a first respective state, in a plurality of states, for the mRNA isoform status for the first respective gene is associated with an improved clinical outcome following treatment of the disease or disorder with a targeted therapy relative to a clinical outcome following treatment of the disease or disorder associated with a second respective state, in the plurality of states, for the mRNA isoform status, with the targeted therapy, further comprising:
when the output of the model indicates the subject has the first respective state for the mRNA isoform status for the first respective gene, administering a first therapeutic regimen comprising the targeted therapy to the subject, and
when the output of the model indicates the subject does not have the first respective state for the mRNA isoform status for the first respective gene, administering a second therapeutic regimen comprising a therapy for the disease or disorder other than the targeted therapy to the subject, wherein the second therapeutic regimen is different than the first therapeutic regimen.

9. (canceled)

10. The method of claim 1, wherein:
the first set of genes comprises a pair of respective genes in the plurality of genes;
the respective plurality of boundary elements comprises, for each respective gene in the pair of respective genes, each corresponding exon-exon boundary element present in one or more mRNA isoforms for the respective gene; and
the genetic status of the subject comprises an indication of whether the subject carries a gene fusion between the pair of respective genes.

11. The method of claim 10, wherein the respective plurality of boundary elements further comprises a set of gene fusion boundary elements for fusions between the pair of respective genes.

12-13. (canceled)

14. The method of claim 10, wherein:
the subject has a disease or disorder; and
treatment of the disease or disorder with a targeted therapy in a patient carrying a gene fusion between the pair of respective genes is associated with an improved clinical outcome relative to a clinical outcome following treatment of the disease or disorder in a patient that does not carry a gene fusion between the pair of respective genes with the targeted therapy, further comprising:
when the output of the model indicates the subject carries a gene fusion between the pair of respective genes, administering the targeted therapy to the subject, and
when the output of the model indicates the subject does not carry a gene fusion between the pair of respective genes, administering a therapy for the disease or disorder other than the targeted therapy to the subject.

15. (canceled)

16. The method of claim 1, wherein:
the respective plurality of boundary elements comprises, for each respective gene in the first set of genes, each exon-exon boundary present in at least one respective mRNA isoform in a plurality of mRNA isoforms for the respective gene; and
the genetic status of the subject comprises a disease state for a disease associated with aberrant mRNA splicing, wherein the disease is cancer, a cardiovascular disease, or a neurological disorder, and the disease state comprises a cancer type, a prognosis for the disease, or a severity of the disease.

17-23. (canceled)

24. The method of claim 1, wherein the one or more genes is at least 25 genes, or wherein the one or more genes represents a whole transcriptome.

25. (canceled)

26. The method of claim 1, wherein the first plurality of nucleic acid sequences were obtained by sequencing cDNA generated from the first plurality of mRNA molecules from the first biological sample.

27. The method of claim 1, wherein the first biological sample of the subject is a solid tumor sample from the subject, a non-cancerous tissue sample from the subject, or a saliva sample or a blood sample from the subject.

28-29. (canceled)

30. The method of claim 1, wherein the obtaining B) comprises:
determining, for each respective nucleic acid sequence in the first plurality of nucleic acid sequences, the respective one or more genes in the plurality of genes corresponding to the respective nucleic acid sequence by mapping the respective nucleic acid sequence to a reference construct representing at least 1 Mb of the genome for the species of the subject,
identifying, for each respective nucleic acid sequence in the plurality of nucleic acid sequences that maps to a respective gene in the first set of genes, each RNA boundary element in the respective plurality of boundary elements that is present in the respective nucleic acid sequence, and
counting, for each respective gene in the first set of genes, the number of occurrences of each respective RNA boundary element in the respective plurality of boundary elements across each respective nucleic acid sequence in the plurality of nucleic acid sequences that maps to a respective gene in the first set of genes, thereby generating a respective abundance value for each respective boundary element in the respective plurality of boundary elements.

31-33. (canceled)

34. The method of claim 1, wherein the corresponding abundance values are determined for each of at least 100 respective RNA boundary elements.

35. The method of claim 1, wherein the model is a statistical inference model, a machine learning model, or a regression model, wherein:
the statistical inference model is a Bayesian inference model, a likelihood-based inference model, a frequentist inference model, an AIC-based inference model, or a mixture model, and
the machine learning model is a support vector regression, a random forest model, an XGBoost model, a Gaussian process model, a deep neural network model, a convolutional neural network model, or a recurrent neural network model.

36-40. (canceled)

41. The method of claim 1, wherein the model processes the first data set, or a plurality of dimensionality reduction components thereof, to determine the genetic status of the subject as an output of the model in N-dimensional space in the applying C), wherein N is a positive integer of at least 4.

42. The method of claim 1, further comprising determining a confidence value for the genetic status of the subject, wherein the confidence value is dependent upon (i) a measure of sequencing depth for the first plurality of nucleic acid sequences, or (ii) the presence or absence of orthogonal evidence for the genetic status.

43-44. (canceled)

45. The method of claim 1, wherein the first data set further comprises one or more features derived from a second plurality of nucleic acid sequences for a first plurality of DNA molecules from a second biological sample of the subject, and wherein the one or more features derived from the second plurality of nucleic acid sequences comprises support for a genomic rearrangement.

46. (canceled)

47. The method of claim 1, wherein the first data set further comprises an indication of a personal characteristic of the subject, wherein:
the personal characteristic of the subject comprises an age, gender, race, ethnicity, smoking status, diabetes status, personal medical history, familial medical history, or a disease state for the subject comprising a cancer type or cancer stage.

48-51. (canceled)

52. A computer system for determining a genetic status of a subject, the computer system comprising:
one or more processors; and
memory addressable by the one or more processors, the memory storing at least one program for execution by the one or more processors, the at least one program comprising instructions for performing a method comprising:
A) obtaining, in electronic form, a first plurality of at least 100,000 nucleic acid sequences for a first plurality of mRNA molecules from a first biological sample of the subject, wherein each mRNA molecule in the first plurality of mRNA molecules corresponds to one or more genes in a plurality of genes;
B) obtaining a first dataset by a process comprising determining, for each respective gene in a first set of genes within the first plurality of genes, a corresponding abundance value for each respective RNA boundary element in a respective plurality of boundary elements of the respective gene in the first plurality of nucleic acid sequences; and
C) applying a model to the first dataset, or a plurality of dimensionality reduction components thereof, thereby determining the genetic status of the subject as output of the model.

53. A non-transitory computer readable storage medium, wherein the non-transitory computer readable storage medium stores instructions, which when executed by a computer system, cause the computer system to perform a method for determining a genetic status of a subject comprising:
A) obtaining, in electronic form, a first plurality of at least 100,000 nucleic acid sequences for a first plurality of mRNA molecules from a first biological sample of the subject, wherein each mRNA molecule in the first plurality of mRNA molecules corresponds to one or more genes in a plurality of genes;
B) obtaining a first dataset by a process comprising determining, for each respective gene in a first set of genes within the first plurality of genes, a corresponding abundance value for each respective RNA boundary element in a respective plurality of boundary elements of the respective gene in the first plurality of nucleic acid sequences; and
C) applying a model to the first dataset, or a plurality of dimensionality reduction components thereof, thereby determining the genetic status of the subject as output of the model.

54-81. (canceled)
